(19) United States
(12) Patent Application Publication
(10) Pub. No.: US 2023/0323410 A1
(43) Pub. Date: Oct. 12, 2023

(54) BACTERIAL CELLS AND METHODS FOR PRODUCTION OF 2-FLUORO-CIS,CIS-MUCONATE

(71) Applicant: Danmarks Tekniske Universitet, Kongens Lyngby (DK)

(72) Inventors: Nicolas Thilo Wirth, Kongens Lyngby (DK); Pablo Ivän Nikel, Kongens Lyngby (DK)

(21) Appl. No.: 18/002,747

(22) PCT Filed: Jul. 2, 2021
(86) PCT No.: PCT/EP2021/068282

§ 371 Date: Dec. 21, 2022

(30) Foreign Application Priority Data
Jul. 2, 2020 (EP) 20183692.1
Apr. 26, 2021 (EP) 21170337.6

(51) Int. Cl. C12P 7/46; C12N 9/02; C12N 15/78

(52) U.S. Cl. CPC C12P 7/46; C12N 9/0071; C12N 9/001; C12N 9/0069; C12N 15/78; C12Y 103/01025; C12Y 113/11001; C12Y 114/1201; C12R 2001/40

(57) ABSTRACT

Herein are disclosed bacterial cells useful for production of 2-fluoro-cis,cis-muconate and derivatives thereof. The disclosure also provides methods and nucleic acid constructs therefor.

### TECHNICAL FIELD

The present invention discloses bacterial cells useful for production of 2-fluoro-cis,cis-muconate and derivatives thereof. The disclosure also provides methods and nucleic acid constructs therefor.

### BACKGROUND

The introduction of fluorine into polymers has been shown to significantly alter their physicochemical properties and can lead to new materials with distinct properties for diverse applications.

Chemical dehydrogenation of cis,cis-muconic acid to adipic acid and subsequent polycondensation with hexamethylenediamine can be used to form the polyamide Nylon 6-6. Nylon 6-6 is frequently used in fibers for textiles and carpets and moulded parts due to its high mechanical strength, rigidity, good stability under heat and/or chemical resistance. It has furthermore broad use in automotive applications, or in electro-insulating elements. The introduction of fluorine into Nylon 6-6 via a chemical synthesis process that uses 2-fluoro-cis,cis-muconate (2-FMA) could alter the polymer properties of Nylon 6-6 significantly, e.g., by increasing its chemical resistance and thermal stability, making it even more suitable for specific applications.

Other applications include the conversion of 2-FMA into 2-fluoro-1,6-hexanediol. The non-halogenated counterpart of this platform molecule (1,6-hexanediol) is widely used for polyester (e.g. PET) and polyurethane production, and as an intermediate in the production of acrylics, adhesives, and dyes. Cis,cis-muconate additionally can serve as one of two precursor compounds to synthesize terephthalic acid, a monomeric chemical to produce polyethylene-terephthalate (PET). Similar beneficial effects as mentioned for fluorinated Nylon 6-6 can be predicted for the incorporation of fluorine into these compounds.

Unfortunately, the fluorinated precursor molecule 2-FMA is not commercially available due to inaccessibility through organic-chemical synthesis. The organic-chemical synthesis of 2-FMA has proved to be highly difficult, if not completely impossible, due to the poisoning of the metal catalyst by fluorinated compounds.

The bioconversion of 3-fluorobenzoic acid (3-FBz) to 2-FMA by wild-type P. putida KT2440 has been shown to occur in shaken-flask experiments with a very low yield and severe growth impairment. In a wild-type strain, the consumption of the substrate 3-FBz proceeds to completion, with a significant accumulation of intermediate metabolites and compounds derived thereof, including non-fluorinated intermediates and byproducts.

Production of 2-FMA has been attempted in biological organisms, however the accumulation of the toxic growth-inhibiting intermediate 3-fluorocatechol has severely limited the yield and efficiency of these processes (Schmidt and Knackmuss, 1984). There is thus a need for reliable and efficient methods for production of fluorinated compounds such as 2-FMA. In particular, sustainable methods based on biological production of fluorinated compounds such as 2-FMA, e.g. from a microorganism such as a bacteria, are needed.

### SUMMARY

The invention is as defined in the claims.

Herein is provided a method of production of 2-fluoro-cis,cis-muconate and derivatives thereof, in a bacterial cell, such as a non-pathogenic Pseudomonas putida cell, from the precursor compound 3-fluorobenzoate. The described process is to the best of our knowledge the first one to efficiently produce 2-fluoro-cis,cis-muconate.

In one aspect, the present disclosure provides a bacterial cell capable of producing 2-fluoro-cis,cis-muconate and optionally derivatives thereof, in the presence of 3-fluorobenzoate or a salt thereof, said bacterial cell expressing:
a native or heterologous benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
a native or heterologous benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
a first, and optionally a second, native or heterologous catechol 1,2-dioxygenases (EC: 1.13.11.1), wherein the first and second catechol 1,2-dioxygenases are each capable of converting 3-fluorocatechol to 2-fluoro-cis,cis-muconate,
   wherein:
the expression of said benzoate 1,2-dioxygenase is under the control of a medium-strong constitutive promoter, whereby the benzoate 1,2-dioxygenase is constitutively overexpressed;
the expression of said first catechol 1,2-dioxygenase is under the control of a first expression module comprising a first promoter, said first expression module further comprising a first strong translation initiation sequence such as a first strong translational coupler, and
the expression of said second catechol 1,2-dioxygenase is under the control of a second expression module comprising a second promoter, said second expression module further comprising a second strong translational initiation sequence such as a second strong translational coupler, whereby the bacterial cell is capable of converting 3-fluorobenzoate to 2-fluoro-cis,cis-muconate.

In one aspect, the present disclosure provides a bacterial cell capable of producing 2-fluoro-cis,cis-muconate and optionally derivatives thereof, in the presence of 3-fluorobenzoate or a salt thereof, said bacterial cell expressing:
a native or heterologous benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
a native or heterologous benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
a first native or heterologous catechol 1,2-dioxygenase (EC: 1.13.11.1), wherein the catechol 1,2-dioxygenase is capable of converting 3-fluorocatechol to 2-fluoro-cis,cis-muconate,
   wherein:
the expression of said benzoate 1,2-dioxygenase is under the control of a medium-strong constitutive promoter, whereby the benzoate 1,2-dioxygenase is constitutively overexpressed; and
the expression of said first catechol 1,2-dioxygenase is under the control of a first expression module comprising a first inducible promoter coupled with a sequence encoding an associated activator protein, said first expression module further comprising a first strong translational coupler, whereby said first catechol 1,2-dioxygenase is expressed in response to 3-fluorobenzoate,
   whereby the bacterial cell is capable of converting 3-fluorobenzoate to 2-fluoro-cis,cis-muconate.

In one aspect, the present disclosure provides a bacterial cell capable of producing 2-fluoro-cis,cis-muconate and optionally derivatives thereof, in the presence of 3-fluorobenzoate or a salt thereof, said bacterial cell expressing:
a native or heterologous benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
a native or heterologous benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
a first and a second native or heterologous catechol 1,2-dioxygenases (EC: 1.13.11.1), wherein the first and second catechol 1,2-dioxygenases are each capable of converting 3-fluorocatechol to 2-fluoro-cis,cis-muconate,
   wherein:
the expression of said benzoate 1,2-dioxygenase is under the control of a medium-strong constitutive promoter, whereby the benzoate 1,2-dioxygenase is constitutively overexpressed;
the expression of said first catechol 1,2-dioxygenase is under the control of a first expression module comprising a first inducible promoter coupled with a sequence encoding an associated activator protein, said first expression module further comprising a first strong translational coupler, whereby said first catechol 1,2-dioxygenase is expressed in response to 3-fluorobenzoate; and
the expression of said second catechol 1,2-dioxygenase is under the control of a second expression module comprising a second inducible promoter, said second expression module further comprising a second strong translational coupler whereby said second catechol 1,2-dioxygenase is expressed in response to 3-fluorobenzoate,
   whereby the bacterial cell is capable of converting 3-fluorobenzoate to 2-fluoro-cis,cis-muconate.

In one aspect, the present disclosure provides a method of producing 2-fluoro-cis,cis-muconate, and optionally derivatives thereof, in a bacterial cell, said method comprising the steps of providing a bacterial cell and incubating said bacterial cell in a medium in the presence of 3-fluorobenzoate or a salt thereof, said bacterial cell expressing:
a native or heterologous benzoate 1,2-dioxygenase (EC: 1.14.12.10);
a native or heterologous benzoate-1,2-dihydrodiol dehydrogenase (EC: 1.3.1.25);
and
a first, and optionally a second, native or heterologous catechol 1,2-dioxygenase (EC: 1.13.11.1),
   wherein:
the expression of said benzoate 1,2-dioxygenase is under the control of a medium-strong constitutive promoter;
the expression of said first catechol 1,2-dioxygenase is under the control of a first expression module comprising a first promoter, said first expression module further comprising a first strong translation initiation sequence such as a first strong translational coupler; and
the expression of said second catechol 1,2-dioxygenase is under the control of a second expression module comprising a second promoter, said second expression module further comprising a second strong translational initiation sequence such as a second strong translational coupler.

In one aspect, the present disclosure provides a method of producing 2-fluoro-cis,cis-muconate, and optionally derivatives thereof, in a bacterial cell, said method comprising the steps of providing a bacterial cell and incubating said bacterial cell in a medium in the presence of 3-fluorobenzoate or a salt thereof, said bacterial cell expressing:
a native or heterologous benzoate 1,2-dioxygenase (EC: 1.14.12.10);
a native or heterologous benzoate-1,2-dihydrodiol dehydrogenase (EC: 1.3.1.25); and
a first native or heterologous catechol 1,2-dioxygenase (EC: 1.13.11.1),
   wherein:
the expression of said benzoate 1,2-dioxygenase is under the control of a medium-strong constitutive promoter;
the expression of said first catechol 1,2-dioxygenase is under the control of a first expression module comprising a first inducible promoter coupled with a sequence encoding an associated activator protein, said first expression module further comprising a first strong translational coupler.

In one aspect, the present disclosure provides a method of producing 2-fluoro-cis,cis-muconate, and optionally derivatives thereof, in a bacterial cell, said method comprising the steps of providing a bacterial cell and incubating said bacterial cell in a medium in the presence of 3-fluorobenzoate or a salt thereof, said bacterial cell expressing:
a native or heterologous benzoate 1,2-dioxygenase (EC: 1.14.12.10);
a native or heterologous benzoate-1,2-dihydrodiol dehydrogenase (EC: 1.3.1.25); and
a first and a second native or heterologous catechol 1,2-dioxygenases (EC: 1.13.11.1),
   wherein:
the expression of said benzoate 1,2-dioxygenase is under the control of a medium-strong constitutive promoter;
the expression of said first catechol 1,2-dioxygenase is under the control of a first expression module comprising a first inducible promoter coupled with a sequence encoding an associated activator protein, said first expression module further comprising a first strong translational coupler; and
the expression of said second catechol 1,2-dioxygenase is under the control of a second expression module comprising a second inducible promoter, said second expression module further comprising a second strong translational coupler.

In one aspect, the present disclosure provides 2-fluoro-cis,cis-muconate or a derivative thereof, preferably wherein the derivative is selected from the group consisting of 2-fluoroadipic acid, [—OC—(F)—(CH₂)₄—CO—NH—(CH₂)₆—NH—]ₙ, 2-fluoro-1,6-hexanediol, a fluorinated polyester, a fluorinated polyurethane, a fluorinated acrylic, a fluorinated adhesive and a fluorinated dye.

In one aspect, the present disclosure provides 2-fluoro-cis,cis-muconate or a derivative thereof obtainable by the methods described herein, preferably wherein the derivative is selected from the group consisting of 2-fluoroadipic acid, [—OC—(F)—(CH₂)₄—CO—NH—(CH₂)₆—NH—]ₙ, 2-fluoro-1,6-hexanediol, a fluorinated polyester, a fluorinated polyurethane, a fluorinated acrylic, a fluorinated adhesive and a fluorinated dye.

In one aspect, the present disclosure provides a nucleic acid construct for modifying a bacterial cell comprising:
a first polynucleotide encoding a native or heterologous benzoate 1,2-dioxygenase (EC: 1.14.12.10) and further comprising a medium-strong constitutive promoter;
a second polynucleotide encoding a native or heterologous benzoate-1,2-dihydrodiol dehydrogenase (EC: 1.3.1.25); and
a third polynucleotide encoding a first native or heterologous catechol 1,2-dioxygenase (EC: 1.13.11.1), a first expression module comprising a first promoter, said first expression module further comprising a first strong translation initiation sequence such as a first strong translational coupler; and
optionally, a fourth polynucleotide encoding a second native or heterologous catechol 1,2-dioxygenase (EC: 1.13.11.1), a second expression module comprising a second promoter, said second expression module further comprising a second strong translational initiation sequence such as a second strong translational coupler.

In one aspect, the present disclosure provides a nucleic acid construct for modifying a bacterial cell comprising:
a first polynucleotide encoding a native or heterologous benzoate 1,2-dioxygenase (EC: 1.14.12.10) and further comprising a medium-strong constitutive promoter;
a second polynucleotide encoding a native or heterologous benzoate-1,2-dihydrodiol dehydrogenase (EC: 1.3.1.25); and
a third polynucleotide encoding a first native or heterologous catechol 1,2-dioxygenase (EC: 1.13.11.1), a first expression module comprising a first inducible promoter and a sequence encoding an associated activator protein, said first expression module further comprising a first strong translational coupler.

In one aspect, the present disclosure provides a nucleic acid construct for modifying a bacterial cell comprising:
a first polynucleotide encoding a native or heterologous benzoate 1,2-dioxygenase (EC: 1.14.12.10) and further comprising a medium-strong constitutive promoter;
a second polynucleotide encoding a native or heterologous benzoate-1,2-dihydrodiol dehydrogenase (EC: 1.3.1.25);
a third polynucleotide encoding a first native or heterologous catechol 1,2-dioxygenase (EC: 1.13.11.1), a first expression module comprising a first inducible promoter and a sequence encoding an associated activator protein, said first expression module further comprising a first strong translational coupler; and
a fourth polynucleotide encoding a second native or heterologous catechol 1,2-dioxygenase (EC: 1.13.11.1), a second expression module comprising a second inducible promoter, said second expression module further comprising a second strong translational coupler.

In another aspect, the present disclosure provides a kit of parts comprising:
the bacterial cell as disclosed herein and instructions for use; and/or
the nucleic acid construct as disclosed herein and instructions for use; and optionally the bacterial cell to be modified.

### DESCRIPTION OF DRAWINGS

FIG. 1A-C shows the results of microtiter plate cultivation. P. knackmussii, P. putida KT2440 and strain Pmuc3 pSEVA228 were cultured in microtiter plates with 200 μl de Bont minimal medium supplemented with 10 g/L MOPS at pH 7.0, 30 mM glucose or potassium benzoate (K-benzoate), as well as varying concentrations of metabolites involved in the bioconversion process.

FIG. 2A-C shows results from a shaken flask fermentation experiment with wildtype P. putida KT2440 and P. knackmussii. Cells were cultured in Erlenmeyer flasks filled with 10% (v/v) de Bont minimal medium supplemented with 30 mM glucose and 10 mM of the respective fluoro-benzoate. (A-B) Time course of extracellular metabolite concentrations and biomass (g_{CDW} L⁻¹). (C) HPLC chromatogram of a calibration standard containing 1 mM of each compound with absorption measured at 280 nm. FIG. 3 shows the bioconversion performance of first generation strains in shaken flask fermentations. All strains were cultured in Erlenmeyer flasks filled with 10% (v/v) de Bont minimal medium supplemented with 30 mM glucose and 10 mM 3-FBz. Shown are the extracellular metabolite concentrations after 24 h of cultivation. The labels represent either the species names for wildtype strains, or the genetic manipulations in engineered P. putida KT2440.

FIG. 4 shows a bioreporter study on the efficiency of bi-cistronic translational coupling sequences (BCD) to initiate mRNA translation. The four BCD variants BCD1, BCD2, BCD7, BCD10, and BCD20 were cloned downstream of the Pm promoter within pSEVA628 derivatives and delivered into P. putida KT2440. The strains were cultured in microtiter plates with each well filled with 200 μl de Bont minimal medium supplemented with 10 g/L MOPS at pH 7.0 and 30 mM glucose

FIG. 5 shows biosensor studies establishing the inducibility of the promoter systems CatR/P_{cat}, BenR/P_{ben} and XylS/Pm. P. putida KT2440 harboring a pSEVA62X plasmid with the msfGFP reporter gene under control of BCD10 as well as either the XylS/Pm system (or Pm variant ML1-17), the native CatR/P_{cat} promoter system, or the BenR/P_{ben} promoter system were cultured in microtiter plates with each well filled with 200 μl de Bont minimal medium supplemented with 10 g/L MOPS at pH 7.0 and 30 mM glucose and varying concentrations of benzoate-derived inducers, as indicated. Fluorescence signals of the reporter strains in mid-exponential growth phase were normalized to bacterial growth and divided by the OD-normalized fluorescence value of the P_{tac} reporter strain.

FIG. 6 shows biosensor studies establishing the relative transcriptional strengths of promoter systems used during strain engineering. P. putida KT2440 harboring a pSEVA62X plasmid with the msfGFP reporter gene under control of BCD10, and transcriptionally regulated by the different variants of the XylS/Pm system, the native CatR/P_{cat} promoter system, the native BenR/P_{ben} promoter system, or the constitutive promoters Pdtac, P_{EM7}, and P_{14g} were cultured in microtiter plates with each well filled with 200 μl de Bont minimal medium supplemented with 10 g/L MOPS at pH 7.0 and 30 mM glucose, as well as 5.0 mM 3-FBz for inducible systems.

FIG. 7 shows the bioconversion performance of second generation strains in shaken flask fermentations. All strains were cultured in Erlenmeyer flasks filled with 10% (v/v) de Bont minimal medium supplemented with 30 mM glucose and 10 mM 3-FBz. Shown are the extracellular metabolite concentrations after 24 h of cultivation. The labels represent either the species names for wildtype strains, or the genetic manipulations in engineered P. putida KT2440. Aliases for selected P. putida strains are written in bold letters.

FIG. 8 shows the HPLC profile of purified 2-FMA. HPLC chromatogram with the absorption measured at 280 nm for a 1.0 mM aqueous solution of 2-FMA that was purified from the fermentation broth of a bioconversion with strain Pmuc3 pSEVA228 and 10 mM 3-FBz.

FIGS. 9A-C show shaken flask bioconversion of 10 mM 3-FBz with strains from the third round of strain engineering.

FIGS. 10A-C show shaken flask bioconversion of 10 mM 3-FBz with strains from the third round of strain engineering.

FIGS. 11A-C show shaken flask bioconversion of 10 mM 3-FBz with strains from the fourth round of strain engineering.

FIGS. 12A-C show shaken flask bioconversion of 10 mM 3-FBz with strains from the fifth round of strain engineering.

FIG. 13 shows the results of 3-FBz toxicity and benzoate assimilation during microtiter plate cultivation in wild-type versus engineered strains. P. putida KT2440 and strain Pmuc10 were cultured in microtiter plates with 200 μl de Bont minimal medium supplemented with 5 g L⁻¹ MOPS at pH 7.0, 30 mM glucose with varying concentrations of 3-FBz, or K-Bz.

FIG. 14 shows the results of 2-FMA toxicity during microtiter plate cultivation. P. putida KT2440 and strain Pmuc10 were cultured in microtiter plates with 200 μl de Bont minimal medium supplemented with 5 g L⁻¹ MOPS at pH 7.0, 30 mM glucose, as well as varying concentrations of 2-FMA.

FIG. 15 shows clustering of promoter systems according to their strength. Concentrations indicated in parentheses (1-2 mM) refer to the inducers 3-methylbenzoate (for XylS/Pm), L-rhamnose (for RhaS-RhaR/Pᵣₕₐ), cyclohexanone (ChnR/P_{chnB}), or 3-chloro-4-hydroxyphenylacetic acid (CprK1/P_{DB3}). Weak promotors comprise sequences that yield a transcriptional strength up to 50% of that provided by P_{tac}. Medium promoters comprise sequences that yield a transcriptional strength from 50% up to 150% of that provided by P_{tac}. Strong promoters comprise sequences that yield a transcriptional strength larger than 150% of that provided by P_{tac}. Actual values of the relative transcription strength (relative to P_{tac}=1) are indicated for each system.

FIG. 16 shows shaken flask bioconversion of 10 mM 3-FBz with strains from the third round of strain engineering. This figure shows data with more biological replicates than FIG. 9A.

### DETAILED DESCRIPTION

Herein is provided a bacterial cell capable of producing 2-fluoro-cis,cis-muconate and derivatives thereof, in a bacterial cell, from the precursor compound 3-fluorobenzoate. The inventors have found that balancing the expression of enzymes encoded by the ben and cat operon in Pseudomonas putida enables efficient conversion of 3-fluorobenzoate to 2-fluoro-cis,cis-muconate with high titers in a bacterial cell, while avoiding the accumulation of the toxic intermediate 3-fluorocatechol. The cell can be engineered as described herein.

Herein are also provided methods for producing 2-fluoro-cis,cis-muconic acid and derivatives thereof. The bacterial cells and nucleic acid constructs described herein are useful for bacterial cell-based production of 2-fluoro-cis,cis-muconic acid and derivatives thereof, including 2-fluoroadipic acid, [—OC—(F)—(CH₂)₄—CO—NH—(CH₂)₆—NH—]ₙ (a fluorinated version of the polyamide Nylon 6-6), 2-fluoro-1,6-hexanediol, fluoro-terephthalic acid, fluoro-trimellitic acid, fluorinated polyesters, fluorinated polyurethanes, fluorinated acrylics, fluorinated adhesives and fluorinated dyes.

Herein is also provided 2-fluoro-cis,cis-muconate or a derivative thereof, preferably wherein the derivative is selected from the group consisting of 2-fluoroadipic acid, [—OC—(F)—(CH₂)₄—CO—NH—(CH₂)₆—NH—]ₙ, 2-fluoro-1,6-hexanediol, a fluorinated polyester, a fluorinated polyurethane, a fluorinated acrylic, a fluorinated adhesive and a fluorinated dye.

Herein is also provided 2-fluoro-cis,cis-muconate or a derivative thereof obtainable by the methods described herein, preferably wherein the derivative is selected from the group consisting of 2-fluoroadipic acid, [—OC—(F)—(CH₂)₄—CO—NH—(CH₂)₆—NH—]ₙ, 2-fluoro-1,6-hexanediol, a fluorinated polyester, a fluorinated polyurethane, a fluorinated acrylic, a fluorinated adhesive and a fluorinated dye.

Herein are also provided nucleic acid constructs for modifying a bacterial cell and useful to perform the present methods.

Also provided herein is a kit of parts comprising a bacterial cell, for example a bacterial cell as described herein, and/or a nucleic acid construct as described herein, and instructions for use.

### Definitions

2-fluoro-cis,cis-muconate is also known as 2-fluoro-cis,cis-muconic acid, 2-fluoro-2,4-hexadienedioic acid or PTYIDKBTGREHJW-TZFCGSKZSA-N. The chemical structure of 2-fluoro-cis,cis-muconate is as shown in formula I:

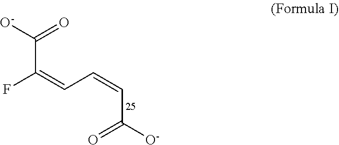 [SMILES: O=C([O-])/C=C\C=C(\F)C(=O)[O-]]

“2-fluoro-cis,cis-muconate derivatives” as used herein refers to compounds obtainable from 2-fluoro-cis,cis-muconate, such as 2-fluoroadipic acid, [—OC—(F)—(CH₂)₄—CO—NH—(CH₂)₆—NH—]ₙ or 2-fluoro-1,6-hexanediol, a fluorinated polyester, a fluorinated polyurethane, a fluorinated acrylic, a fluorinated adhesive and a fluorinated dye. The term “derivative” in this context thus refers to compounds which can be obtained by converting 2-fluoro-cis,cis-muconate into another compound, such as the previously listed compounds.

Functional variant: the term is herein applied to functional variants of enzymes, i.e. modified versions of the enzyme, or homologous enzymes originating from a different species, which retain some or all the catalytic activity of the original enzyme. Functional variants may have been modified by introducing mutations which confer e.g. increased activity, a change in intracellular localisation, prolonged half-life, among others, but retain the ability to perform the same enzymatic reaction as the enzymes they are derived from, albeit possibly to a different extent. Activities of functional variants may be determined as described elsewhere herein.

“Identity”, “similarity” and “homology” with respect to a polynucleotide (or polypeptide) is defined herein as the percentage of nucleic acids (or amino acids) in the candidate sequence that are identical with the residues of a corresponding native nucleic acids (or amino acids), after aligning the sequences and introducing gaps, if necessary, to achieve the maximum percent identity/similarity/homology, and considering any conservative substitutions according to the NCIUB rules (hftp://www.chem.qmul.ac.uk/iubmb/misc/naseq.html; NC-IUB, Eur J Biochem (1985) 150: 1-5) as part of the sequence identity. Neither 5′ or 3′ extensions nor insertions result in a reduction of identity, similarity or homology. Methods and computer programs for the alignments are well known in the art.

By “encoding” or “encoded”, in the context of a specified nucleic acid, is meant comprising the information for translation into the specified protein. A nucleic acid or polynucleotide encoding a protein may comprise non-translated sequences, e.g. introns, within translated regions of the nucleic acid, or may lack such intervening non-translated sequences, e.g. in cDNA. The information by which a protein is encoded is specified by the use of codons.

The term “gene” means the segment of DNA involved in producing a polypeptide chain; it includes regions preceding and following the coding region (promoter and terminator).

As used herein, “expression” in the context of nucleic acids is to be understood as the transcription and accumulation of sense mRNA or antisense RNA derived from a nucleic acid fragment. “Expression” used in the context of proteins refers to translation of mRNA into a polypeptide.

The term “overexpression” as used herein refers to a process by which a gene comprising a sequence that encodes a polypeptide is artificially expressed in a modified cell to produce a level of expression of the encoded polypeptide that exceeds the level of expression of the same polypeptide in an unmodified, or in a reference host cell. Thus, while the term is typically used in conjunction with a gene, the term “overexpression” may also be used in conjunction with a protein to refer to the increased level of a protein resulting from the overexpression of its encoding gene. In some embodiments, overexpression of a gene encoding a protein is achieved by increasing the number of copies of the gene that encodes the protein. In other embodiments, overexpression of a gene encoding a protein is achieved by increasing the binding strength of the promoter region and/or the ribosome binding site in such a way to increase the transcription and/or the translation of the gene that encodes the protein. In other embodiments, overexpression can be achieved by increasing the number of copies of a gene and increasing the binding strength of the promoter region and/or the ribosome binding site. In some embodiments, the overexpression of a gene encoding a protein results from the expression of at least one copy of the corresponding encoding polynucleotide present on a multicopy plasmid that has been introduced into a host cell. In other embodiments, the overexpression of a gene encoding a protein results from the expression of two or more copies of the corresponding encoding polynucleotide that are integrated into the genome of the host cell. For expression of heterologous proteins where no native expression level exists for comparison, “overexpression” refers to expressing the protein at a medium or strong level in the cell.

“Polycistronic” as defined herein describes a type of messenger RNA that can encode more than one polypeptide separately within the same RNA molecule. Bacterial messenger RNA (mRNA) is generally polycistronic.

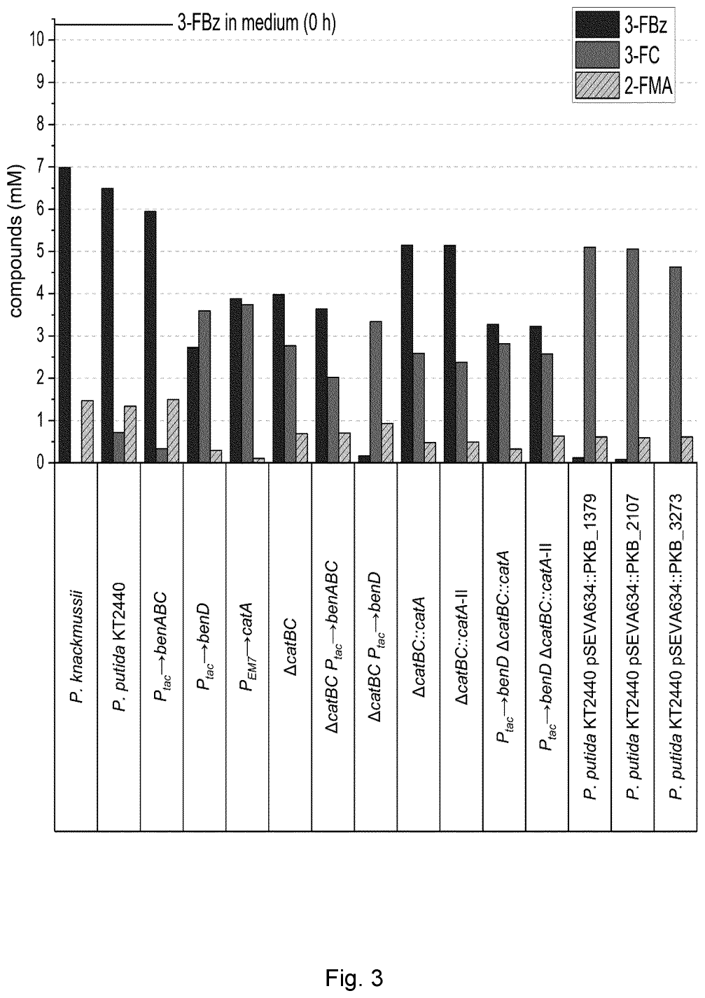

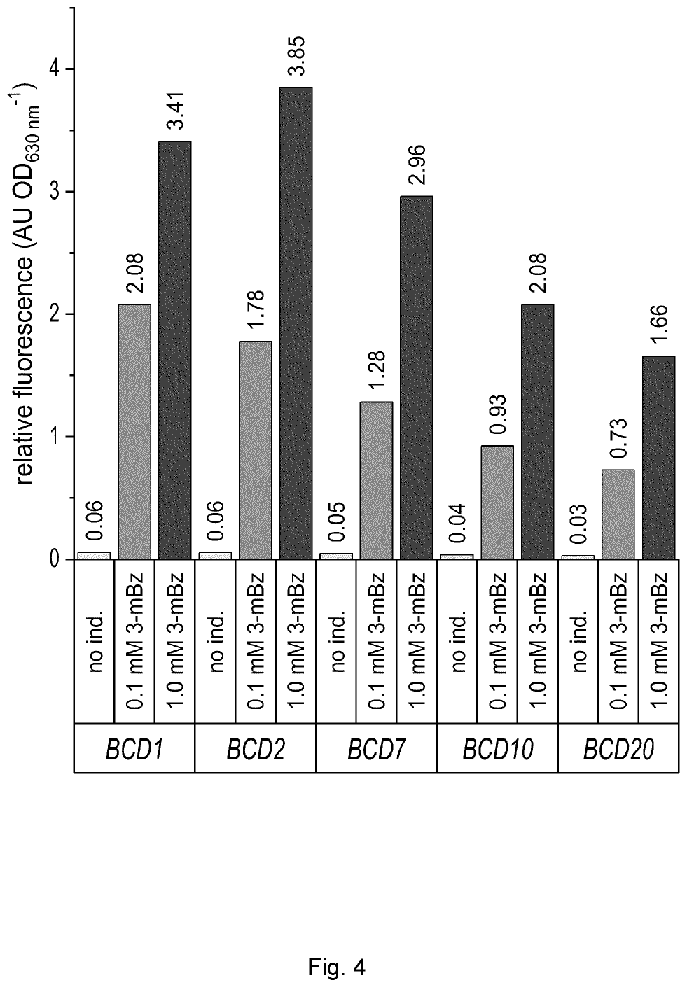

“Translational coupling” as defined herein refers to the interdependence of translation efficiency of neighbouring genes encoded within an operon. The degree of coupling may be quantified by measuring how the translation rate of a gene is modulated by the translation rate of its upstream gene. The polycistronic mRNAs of bacteria enable translational coupling of two or more genes, whereby translation of a downstream gene depends on the translation of the upstream gene. A “strong translational coupler” as defined herein denotes a translational coupler whose expression strength value is within the highest 20% of synthetic translation initiation sequences developed and characterized by Mutalik et al. (2013) and, in the case of Pseudomonas species, as defined by Zobel et al. (2015). The strength of translational couplers in combination with different promoters has been tested using fluorescent proteins as described by Zobel et al. (2015). Zobel et al. characterized a promotor library using the same translational coupler (BCD2, the strongest one) as reference. Different BCD sequences were characterized in P. putida in the present disclosure (See FIG. 4), and found to have similar relative strengths as originally published by Mutalik et al. (2013) for E. coli. “Translation initiation strength” as used herein refers to the translation initiation rate of a given ribonucleic acid sequence, i.e. at what probability a ribosome would initiate translation at a particular locus when scanning the given sequence. The calculation of such a translation initiation rate for a given nucleic acid sequence and the design of such a sequence with a target translation initiation rate is generally known in the art. An example includes the Ribosome Binding Site (RBS) calculator available online at https://salislab.net/software/design_rbs_calculator.

An “inducible promoter” in the context of the present disclosure refers to a promoter which is regulated or activated under certain conditions, such as light, chemical concentration, protein concentration, conditions in an organism, cell, or organelle, etc. An example of an inducible promoter is the Pm promoter from Pseudomonas putida mt-2 which is controlled by the activator protein XylS that responds to a range of benzoic acid derivatives by inducing expression of the gene under the control of the Pm promoter. In essence, expression under the control of an inducible promoter is “switched on” or increased in response to an applied stimulus. The nature of the stimulus varies between promoters and may include the above environmental factors. Whatever the level of expression is in the absence of the stimulus, expression from any inducible promoter is increased in the presence of the correct stimulus. The term “constitutive promoter” may be understood as an extensively unregulated promoter that allows continual transcription of its associated gene.

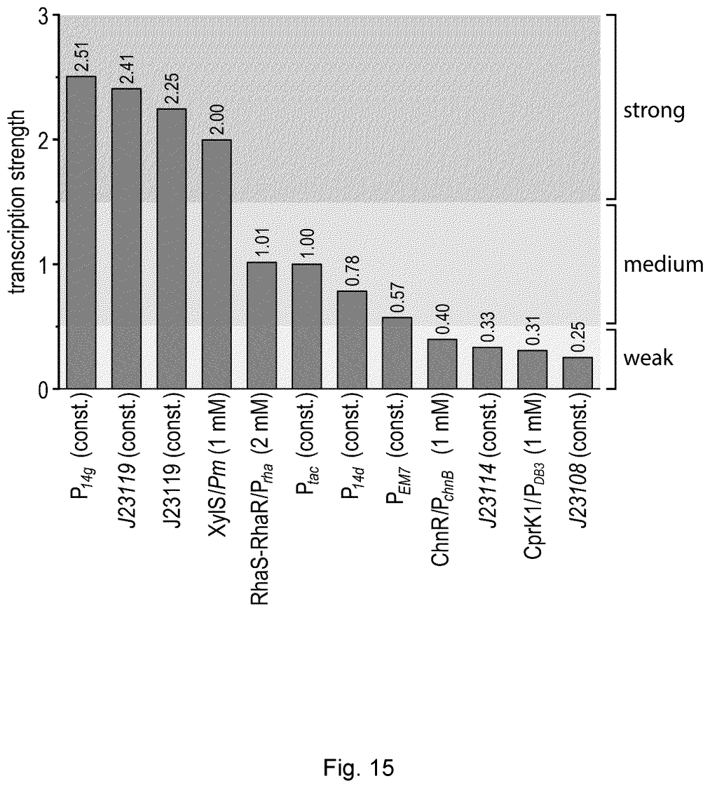

Classification of promoter strength as “weak”, “medium-strength” or “strong” refers to the degree to which the promoter alters the rate of transcription initiation from its associated gene compared to the endogenous level. This can result in either an increase or a decrease in the associated gene transcription compared to endogenous levels. The skilled person knows whether a given promoter is classified as a weak, medium-strength or strong promoter. Further examples of promoters and promoter systems and their classification as weak, medium-strength or strong are shown in FIG. 15.

BenABC: benzoate 1,2-dioxygenase (EC: 1.14.12.10)

benzoate+NADH+H⁺+O₂<=>cyclohexadiene-cis,cis-1,2-diol-1-carboxylate+NAD⁺

The enzyme catalyzes conversion of a benzoate to the corresponding cyclohexadiene-cis,cis-1,2-diol-1-carboxylate in the presence of NADH, H⁺ and oxygen. For example it catalyzes the conversion of 3-fluorobenzoate into 3-fluorocyclohexadiene-cis,cis-1,2-diol-1-carboxylate. Benzoate 1,2-dioxygenase activity can be determined spectrophotometrically by measuring the decrease in absorbance at 340 nm of NADH or polarographically by measuring the oxygen consumption (Yamaguchi and Fujisawa al., 1980).

BenD: benzoate-1,2-dihydrodiol dehydrogenase (EC: 1.3.1.25)

cyclohexadiene-cis,cis-1,2-diol-1-carboxylate+NAD+<=>catechol+CO₂+NADH+H⁺

The enzyme catalyzes conversion of a cyclohexadiene-cis,cis-1,2-diol-1-carboxylate to the corresponding catechol in the presence of NAD⁺. For example it catalyzes the conversion of 3-fluorocyclohexadiene-cis,cis-1,2-diol-1-carboxylate into 3-fluorocatechol. Benzoate-1,2-dihydrodiol dehydrogenase activity can be determined spectrophotometrically by measuring the increase in absorbance at 340 nm of NADH (Reiner, 1972).

CatA-I: catechol 1,2-dioxygenase (EC: 1.13.11.1)

catechol+O2<=>cis,cis-muconate

The enzyme catalyzes conversion of a catechol to the corresponding cis,cis-muconate in the presence of oxygen. For example it catalyzes the conversion of 3-fluorocatechol into 2-fluoro-cis,cis-muconate. Catechol 1,2-dioxygenase activity can be determined spectrophotometrically by monitoring the increase in cis,cis-muconate concentration at 260 nm (Wojcieszyńska et al., 2011).

CatA-II: catechol 1,2-dioxygenase (EC: 1.13.11.1)

catechol+O2<=>cis,cis-muconate

The enzyme catalyzes conversion of a catechol to the corresponding cis,cis-muconate in the presence of oxygen. For example it catalyzes the conversion of 3-fluorocatechol into 2-fluoro-cis,cis-muconate. Catechol 1,2-dioxygenase activity can be determined spectrophotometrically by monitoring the increase in cis,cis-muconate concentration at 260 nm (Wojcieszyńska et al., 2011).

### Bacterial Cell

The present disclosure relates to a bacterial cell capable of producing 2-fluoro-cis,cis-muconate and derivatives thereof, in a bacterial cell, from the precursor compound 3-fluorobenzoate. The inventors have found that balancing the expression of enzymes encoded by the ben and cat operon in Pseudomonas putida using suitable promoters as described herein in detail enables efficient conversion of 3-fluorobenzoate to 2-fluoro-cis,cis-muconate with high titers in a bacterial cell, while avoiding the accumulation of the toxic intermediate 3-fluorocatechol as well as the toxic effects of catechol 1,2-dioxygenase overexpression. The cell can be engineered as described herein.

Herein is provided a bacterial cell capable of producing 2-fluoro-cis,cis-muconate and optionally derivatives thereof, in the presence of 3-fluorobenzoate or a salt thereof, said bacterial cell expressing:
a native or heterologous benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
a native or heterologous benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
a first, and optionally a second, native or heterologous catechol 1,2-dioxygenases (EC: 1.13.11.1), wherein the first and second catechol 1,2-dioxygenases are each capable of converting 3-fluorocatechol to 2-fluoro-cis,cis-muconate,
   wherein:
the expression of said benzoate 1,2-dioxygenase is under the control of a medium-strong constitutive promoter, whereby the benzoate 1,2-dioxygenase is constitutively overexpressed;
the expression of said first catechol 1,2-dioxygenase is under the control of a first expression module comprising a first promoter, said first expression module further comprising a first strong translation initiation sequence such as a first strong translational coupler, and
the expression of said second catechol 1,2-dioxygenase is under the control of a second expression module comprising a second promoter, said second expression module further comprising a second strong translational initiation sequence such as a second strong translational coupler,
   whereby the bacterial cell is capable of converting 3-fluorobenzoate to 2-fluoro-cis,cis-muconate.

Herein is also provided a bacterial cell capable of producing 2-fluoro-cis,cis-muconate and optionally derivatives thereof, in the presence of 3-fluorobenzoate or a salt thereof, said bacterial cell expressing:
a native or heterologous benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
a native or heterologous benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
a first native or heterologous catechol 1,2-dioxygenase (EC: 1.13.11.1), wherein the catechol 1,2-dioxygenase is capable of converting 3-fluorocatechol to 2-fluoro-cis,cis-muconate,
   wherein:
the expression of said benzoate 1,2-dioxygenase is under the control of a medium-strong constitutive promoter, whereby the benzoate 1,2-dioxygenase is constitutively overexpressed; and
the expression of said first catechol 1,2-dioxygenase is under the control of a first expression module comprising a first inducible promoter coupled with a sequence encoding an associated activator protein, said first expression module further comprising a first strong translational coupler, whereby said first catechol 1,2-dioxygenase is expressed in response to 3-fluorobenzoate,
   whereby the bacterial cell is capable of converting 3-fluorobenzoate to 2-fluoro-cis,cis-muconate.

In some bacterial cells, more than one of each of the before-mentioned classes of enzymes may be expressed. Thus in some cases, the cell may contain more than one benzoate 1,2-dioxygenase, benzoate-1,2-dihydrodiol dehydrogenase and/or catechol 1,2-dioxygenase.

In some embodiments, the present disclosure provides a bacterial cell capable of producing 2-fluoro-cis,cis-muconate and optionally derivatives thereof, in the presence of 3-fluorobenzoate or a salt thereof, said bacterial cell expressing:
a native or heterologous benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
a native or heterologous benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
a first and a second native or heterologous catechol 1,2-dioxygenases (EC: 1.13.11.1), wherein the first and second catechol 1,2-dioxygenases are each capable of converting 3-fluorocatechol to 2-fluoro-cis,cis-muconate,
   wherein:
the expression of said benzoate 1,2-dioxygenase is under the control of a medium-strong constitutive promoter, whereby the benzoate 1,2-dioxygenase is constitutively overexpressed;
the expression of said first catechol 1,2-dioxygenase is under the control of a first expression module comprising a first inducible promoter coupled with a sequence encoding an associated activator protein, said first expression module further comprising a first strong translational coupler, whereby said first catechol 1,2-dioxygenase is expressed in response to 3-fluorobenzoate; and
the expression of said second catechol 1,2-dioxygenase is under the control of a second expression module comprising a second inducible promoter, said second expression module further comprising a second strong translational coupler whereby said second catechol 1,2-dioxygenase is expressed in response to 3-fluorobenzoate,
   whereby the bacterial cell is capable of converting 3-fluorobenzoate to 2-fluoro-cis,cis-muconate.

In some embodiments, the bacterial cell is a non-pathogenic organism.

In some embodiments, the bacterial cell can tolerate toxic compounds, such as fluorinated compounds.

In some embodiments, the bacterial cell belongs to the Pseudomonas genus, the Burkholderia genus, the Escherichia genus, the Rhodococcus genus, the Bacillus genus or the Vibrio genus.

In some embodiments, the bacterial cell may be selected from the group Pseudomonas putida, Pseudomonas knackmussi, Pseudomonas fluorescens, Pseudomonas taiwanesis, Pseudomonas syringae, Pseudomonas stutzeri, Pseudomonas oleovorans, Pseudomonas mendocina, Burkholderia fungorum, Escherichia coli, Rhodococcus rhodochrous, Bacillus subtilis, Bacillus cereus, Bacillus megaterium and Vibrio natriegens. In preferred embodiments, the bacterial cell is a Pseudomonas putida cell, such as a Pseudomonas putida KT2440 cell.

The present bacterial cell requires the precursor molecule 3-fluorobenzoate or a salt thereof in order to produce 2-fluoro-cis,cis-muconate. 3-fluorobenzoate or a salt thereof may be provided directly in the medium or it may be derived from a precursor molecule through a metabolic process by the bacterial cell; said precursor molecule may be synthesized by the cell, and/or it may be provided in the medium.

Production of 2-Fluoro-Cis,Cis-Muconate

The bacterial cell of the present disclosure can produce 2-fluoro-cis,cis-muconate. This requires that the bacterial cell expresses a benzoate 1,2-dioxygenase capable of converting 3-fluorobenzoate to 3-fluorocyclohexadiene-cis,cis-1,2-diol-1-carboxylate, a benzoate-1,2-dihydrodiol dehydrogenase capable of converting 3-fluorocyclohexadiene-cis,cis-1,2-diol-1-carboxylate to 3-fluorocatechol, and a catechol 1,2-dioxygenase capable of converting 3-fluorocatechol to 2-fluoro-cis,cis-muconate.

A benzoate 1,2-dioxygenase (EC: 1.3.1.25) catalyzes the conversion of a benzoate to the corresponding cyclohexadiene-cis,cis-1,2-diol-1-carboxylate in the presence of NADH, H⁺ and oxygen according to the following reaction:

benzoate+NADH+H⁺+O₂<=>cyclohexadiene-cis,cis-1,2-diol-1-carboxylate+NAD⁺

For example, the benzoate 1,2-dioxygenase may catalyze the conversion of 3-fluorobenzoate into 3-fluorocyclohexadiene-cis,cis-1,2-diol-1-carboxylate. The benzoate 1,2-dioxygenase activity of an enzyme may be tested as per the procedure of Yamaguchi & Fujisawa (1980). A functional variant of a benzoate 1,2-dioxygenase may be identified by testing if it retains some or all of the benzoate 1,2-dioxygenase catalytic activity when tested as described herein.

A benzoate-1,2-dihydrodiol dehydrogenase (EC: 1.3.1.25) catalyzes conversion of a cyclohexadiene-cis,cis-1,2-diol-1-carboxylate to the corresponding catechol in the presence of NAD⁺ according to the following reaction:

cyclohexadiene-cis,cis-1,2-diol-1-carboxylate+NAD⁺<=>catechol+CO₂+NADH+H⁺

For example, the benzoate-1,2-dihydrodiol dehydrogenase may catalyze the conversion of 3-fluorocyclohexadiene-cis,cis-1,2-diol-1-carboxylate into 3-fluorocatechol. The benzoate-1,2-dihydrodiol dehydrogenase activity of an enzyme may be tested as described by Reiner (1972). A functional variant of a benzoate-1,2-dihydrodiol dehydrogenase may be identified by testing if it retains some or all of the benzoate-1,2-dihydrodiol dehydrogenase catalytic activity when tested as described herein.

A catechol 1,2-dioxygenase (EC: 1.13.11.1) catalyzes the conversion of a catechol to the corresponding cis,cis-muconate in the presence of oxygen according to the following reaction:

catechol+O2<=>cis,cis-muconate

For example, the catechol 1,2-dioxygenase may catalyze the conversion of 3-fluorocatechol into 2-fluoro-cis,cis-muconate. The catechol 1,2-dioxygenase activity of an enzyme may be tested by the procedure of Wojcieszyńska et al. (2011). A functional variant of a catechol 1,2-dioxygenase may be identified by testing if it retains some or all of the catechol 1,2-dioxygenase catalytic activity when tested as described herein.

In some embodiments, the benzoate 1,2-dioxygenase is a native or heterologous benzoate 1,2-dioxygenase. In some embodiments, the benzoate 1,2-dioxygenase is BenABC or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. The benzoate 1,2-dioxygenase is capable of converting 3-fluorobenzoate to 3-fluorocyclohexadiene-cis,cis-1,2-diol-1-carboxylate. In some embodiments, the benzoate 1,2-dioxygenase comprises:
i) BenA (SEQ ID NO: 1),
ii) BenB (SEQ ID NO: 2), and
iii) BenC (SEQ ID NO: 3)
   or functional variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the benzoate 1,2-dioxygenase consists of BenA, BenB and BenC or functional variants thereof having at least 80% homology or identity thereto.

In some preferred embodiments, the benzoate 1,2-dioxygenase originates from the organism Pseudomonas putida.

In some embodiments, the benzoate 1,2-dioxygenase, for example a benzoate 1,2-dioxygenase consisting of BenA, BenB and BenC or functional variants thereof having at least 80% homology or identity thereto, is expressed under the control of a medium-strong constitutive promoter, whereby the benzoate 1,2-dioxygenase is constitutively overexpressed. In some embodiments, the medium-strong constitutive promoter is P_{tac} (SEQ ID NO: 15) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments, the native promoter of the benzoate 1,2-dioxygenase, such as the Pben promoter (SEQ ID NO: 13) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, has been replaced with a medium-strong constitutive promoter as described herein above.

In some embodiments, the medium-strong constitutive promoter is P_{14g} (SEQ ID NO: 50) or a variant thereof having at least 80%, such as at least 85%, such as at least 90%, such as at least 95%, such as at least 99% homology or identity thereto. In some embodiments, the medium-strong constitutive promoter is P_{14f} (SEQ ID NO: 53) or a variant thereof having at least 80%, such as at least 85%, such as at least 90%, such as at least 95%, such as at least 99% homology or identity thereto. In some embodiments, the medium-strong constitutive promoter is P₁₄ₑ (SEQ ID NO: 54) or a variant thereof having at least 80%, such as at least 85%, such as at least 90%, such as at least 95%, such as at least 99% homology or identity thereto. In some embodiments, the medium-strong constitutive promoter is P_{14d} (SEQ ID NO: 55) or a variant thereof having at least 80%, such as at least 85%, such as at least 90%, such as at least 95%, such as at least 99% homology or identity thereto. In some embodiments, the medium-strong constitutive promoter is BG19 (SEQ ID NO: 56) or a variant thereof having at least 80%, such as at least 85%, such as at least 90%, such as at least 95%, such as at least 99% homology or identity thereto. In some embodiments, the medium-strong constitutive promoter is BG34 (SEQ ID NO: 57) or a variant thereof having at least 80%, such as at least 85%, such as at least 90%, such as at least 95%, such as at least 99% homology or identity thereto. In some embodiments, the medium-strong constitutive promoter is P_{14c} (SEQ ID NO: 58) or a variant thereof having at least 80%, such as at least 85%, such as at least 90%, such as at least 95%, such as at least 99% homology or identity thereto. In some embodiments, the medium-strong constitutive promoter is P_{EM7} (SEQ ID NO: 59) or a variant thereof having at least 80%, such as at least 85%, such as at least 90%, such as at least 95%, such as at least 99% homology or identity thereto. In some embodiments, the medium-strong constitutive promoter is J23119 (SEQ ID NO: 60) or a variant thereof having at least 80%, such as at least 85%, such as at least 90%, such as at least 95%, such as at least 99% homology or identity thereto. In some embodiments, the medium-strong constitutive promoter is J23101 (SEQ ID NO: 61) or a variant thereof having at least 80%, such as at least 85%, such as at least 90%, such as at least 95%, such as at least 99% homology or identity thereto. In some embodiments, the medium-strong constitutive promoter is J23107 (SEQ ID NO: 62) or a variant thereof having at least 80%, such as at least 85%, such as at least 90%, such as at least 95%, such as at least 99% homology or identity thereto. In some embodiments, the medium-strong constitutive promoter is JEc3 (SEQ ID NO: 63) or a variant thereof having at least 80%, such as at least 85%, such as at least 90%, such as at least 95%, such as at least 99% homology or identity thereto. In some embodiments, the medium-strong constitutive promoter is JEa3 (SEQ ID NO: 64) or a variant thereof having at least 80%, such as at least 85%, such as at least 90%, such as at least 95%, such as at least 99% homology or identity thereto. In some embodiments, the medium-strong constitutive promoter is JE1611 (SEQ ID NO: 65) or a variant thereof having at least 80%, such as at least 85%, such as at least 90%, such as at least 95%, such as at least 99% homology or identity thereto. In some embodiments, the medium-strong constitutive promoter is JEa2 (SEQ ID NO: 66) or a variant thereof having at least 80%, such as at least 85%, such as at least 90%, such as at least 95%, such as at least 99% homology or identity thereto.

In some embodiments, the translation initiation region of the gene encoding the benzoate 1,2-dioxygenase, for example the benzoate 1,2-dioxygenase consisting of BenA, BenB and BenC or functional variants thereof having at least 80% homology or identity thereto, is unmodified. In some embodiments, the translation initiation region of the gene encoding the benzoate 1,2-dioxygenase subunit BenA described herein above is unmodified. In some embodiments, the translation initiation region consisting of the 34 base pairs upstream from the start codon of said gene comprises or consists of SEQ ID NO: 24 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

Any suitable nucleic sequence may be used to replace the translation initiation region of the gene encoding the benzoate 1,2-dioxygenase subunit BenA. In some embodiments, the translation initiation region consisting of the 34 base pairs upstream from the start codon of the gene encoding benzoate 1,2-dioxygenase subunit BenA comprises or consists of a translation initiation region sequence with a predicted translation rate of 100 to 5,000, predicted using the RBS Calculator available online at https://salislab.net/software/design_rbs_calculator.

In some embodiments, the translation initiation region of the gene encoding the benzoate 1,2-dioxygenase subunit BenA comprises or consists of BCD22 (SEQ ID NO: 68) or a variant thereof having at least 80%, such as at least 85%, such as at least 90%, such as at least 95%, such as at least 99% homology or identity thereto. In some embodiments, the translation initiation region of the gene encoding the benzoate 1,2-dioxygenase subunit BenA comprises or consists of BCD16 (SEQ ID NO: 69) or a variant thereof having at least 80%, such as at least 85%, such as at least 90%, such as at least 95%, such as at least 99% homology or identity thereto. In some embodiments, the translation initiation region of the gene encoding the benzoate 1,2-dioxygenase subunit BenA comprises or consists of BCD23 (SEQ ID NO: 70) or a variant thereof having at least 80%, such as at least 85%, such as at least 90%, such as at least 95%, such as at least 99% homology or identity thereto. In some embodiments, the translation initiation region of the gene encoding the benzoate 1,2-dioxygenase subunit BenA comprises or consists of BCD8 (SEQ ID NO: 71) or a variant thereof having at least 80%, such as at least 85%, such as at least 90%, such as at least 95%, such as at least 99% homology or identity thereto. In some embodiments, the translation initiation region of the gene encoding the benzoate 1,2-dioxygenase subunit BenA comprises or consists of BCD21 (SEQ ID NO: 72) or a variant thereof having at least 80%, such as at least 85%, such as at least 90%, such as at least 95%, such as at least 99% homology or identity thereto. In some embodiments, the translation initiation region of the gene encoding the benzoate 1,2-dioxygenase subunit BenA comprises or consists of MCD18 (SEQ ID NO: 73) or a variant thereof having at least 80%, such as at least 85%, such as at least 90%, such as at least 95%, such as at least 99% homology or identity thereto. In some embodiments, the translation initiation region of the gene encoding the benzoate 1,2-dioxygenase subunit BenA comprises or consists of MCD8 (SEQ ID NO: 74) or a variant thereof having at least 80%, such as at least 85%, such as at least 90%, such as at least 95%, such as at least 99% homology or identity thereto. In some embodiments, the translation initiation region of the gene encoding the benzoate 1,2-dioxygenase subunit BenA comprises or consists of MCD14 (SEQ ID NO: 75) or a variant thereof having at least 80%, such as at least 85%, such as at least 90%, such as at least 95%, such as at least 99% homology or identity thereto. In some embodiments, the translation initiation region of the gene encoding the benzoate 1,2-dioxygenase subunit BenA comprises or consists of MCD15 (SEQ ID NO: 76) or a variant thereof having at least 80%, such as at least 85%, such as at least 90%, such as at least 95%, such as at least 99% homology or identity thereto. In some embodiments, the translation initiation region of the gene encoding the benzoate 1,2-dioxygenase subunit BenA comprises or consists of MCD22 (SEQ ID NO: 77) or a variant thereof having at least 80%, such as at least 85%, such as at least 90%, such as at least 95%, such as at least 99% homology or identity thereto.

In some embodiments, the benzoate-1,2-dihydrodiol dehydrogenase (EC: 1.3.1.25) is a native or heterologous benzoate-1,2-dihydrodiol dehydrogenase. In some embodiments, the benzoate-1,2-dihydrodiol dehydrogenase is BenD (SEQ ID NO: 7) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some preferred embodiments, the benzoate-1,2-dihydrodiol dehydrogenase is native to Pseudomonas putida.

In some embodiments, the translation initiation region and/or the promoter of the gene encoding the benzoate-1,2-dihydrodiol dehydrogenase, for example BenD, is unmodified. In some embodiments, the translation initiation region and/or the promoter of the gene encoding the benzoate-1,2-dihydrodiol dehydrogenase BenD described herein above is unmodified. In some embodiments, the genomic region upstream of said gene containing said translation initiation region and/or promoter comprises or consists of SEQ ID NO: 25 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the bacterial cell expresses at least a first native or heterologous catechol 1,2-dioxygenase (EC: 1.13.11.1). In some embodiments, the first catechol 1,2-dioxygenase is CatA-I (SEQ ID NO: 9) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some preferred embodiments, the first catechol 1,2-dioxygenase is native to the organism Pseudomonas putida.

In some embodiments, the first catechol 1,2-dioxygenase, for example CatA-I, is expressed under the control of a first expression module comprising a first promoter, said first expression module further comprising a first strong translation initiation sequence such as a first strong translational coupler.

In some embodiments, the first promoter of the first expression module is a medium-to-weak constitutive promoter, such as P_{14b} (SEQ ID NO: 49), whereby the catechol 1,2-dioxygenase is constitutively expressed.

In some embodiments, the first catechol 1,2-dioxygenase, for example CatA-I, is expressed under the control of a first expression module comprising a medium-to-weak constitutive promoter, said first expression module further comprising a first strong translational coupler, whereby said catechol 1,2-dioxygenase is constitutively expressed. In some embodiments, the medium-to-weak constitutive promoter of said first expression module is P_{14b} (SEQ ID NO: 49), and the first strong translational coupler is BCD2 (SEQ ID NO: 22) or BCD10 (SEQ ID NO: 16), or variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, preferably the first strong translational coupler is BCD10 (SEQ ID NO: 16) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the first promoter of the first expression module is a first inducible promoter, optionally coupled with a sequence encoding an associated activator protein.

In some embodiments, the first catechol 1,2-dioxygenase, for example CatA-I, is expressed under the control of a first expression module comprising a first inducible promoter coupled with a sequence encoding an associated activator protein, said first expression module further comprising a first strong translational coupler, whereby said catechol 1,2-dioxygenase is expressed strongly in response to 3-fluorobenzoate. In other words, when 3-fluorobenzoate is present in the media, the strong translational coupler ensures that the expression change mediated by the inducible promoter strongly correlates with the amount of subsequently transcribed mRNA that is translated into protein. The first expression module thus ensures that the cell comprises many molecules of catechol 1,2-dioxygenase only when 3-fluorobenzoate is present. In some embodiments, the first inducible promoter of said first expression module is the Pm variant ML1-17 (SEQ ID NO: 18) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, the associated activator protein is XylS (SEQ ID NO: 21) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, and the first strong translational coupler is BCD2 (SEQ ID NO: 22) or BCD10 (SEQ ID NO: 16), or variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, preferably the first strong translational coupler is BCD10 (SEQ ID NO: 16) or a variant thereof having at least 80% homology or identity thereto.

In some embodiments the first strong translational coupler is BCD11 (SEQ ID NO: 44), BCD7 (SEQ ID NO: 45), BCD6 (SEQ ID NO: 17), BCD5 (SEQ ID NO: 19), or BCD1 (SEQ ID NO: 23) or variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the bacterial cell expresses at least a first and a second native or heterologous catechol 1,2-dioxygenases (EC: 1.13.11.1). In some embodiments, the first catechol 1,2-dioxygenase is CatA-I (SEQ ID NO: 9), and the second catechol 1,2-dioxygenase is CatA-II (SEQ ID NO: 11), or functional variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some preferred embodiments, the first and the second catechol 1,2-dioxygenases are native to Pseudomonas putida.

In some embodiments, the expression of the first catechol 1,2-dioxygenase, for example CatA-I, described herein is under the control of a first expression module comprising a first promoter, said first expression module further comprising a first strong translation initiation sequence such as a first strong translational coupler, and the expression of said second catechol 1,2-dioxygenase is under the control of a second expression module comprising a second promoter, said second expression module further comprising a second strong translational initiation sequence such as a second strong translational coupler.

In some embodiments, the first and/or the second promoter is a medium-to-weak constitutive promoter, whereby the catechol 1,2-dioxygenase is constitutively expressed. Thus, in some embodiments, the first promoter is a first medium-to-weak constitutive promoter. In some embodiments, the second promoter is a second medium-to-weak constitutive promoter. In some embodiments, the first and the second promoters are medium-to-weak constitutive promoters.

In some embodiments, the medium-to-weak constitutive promoter is P_{14b} (SEQ ID NO: 49) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

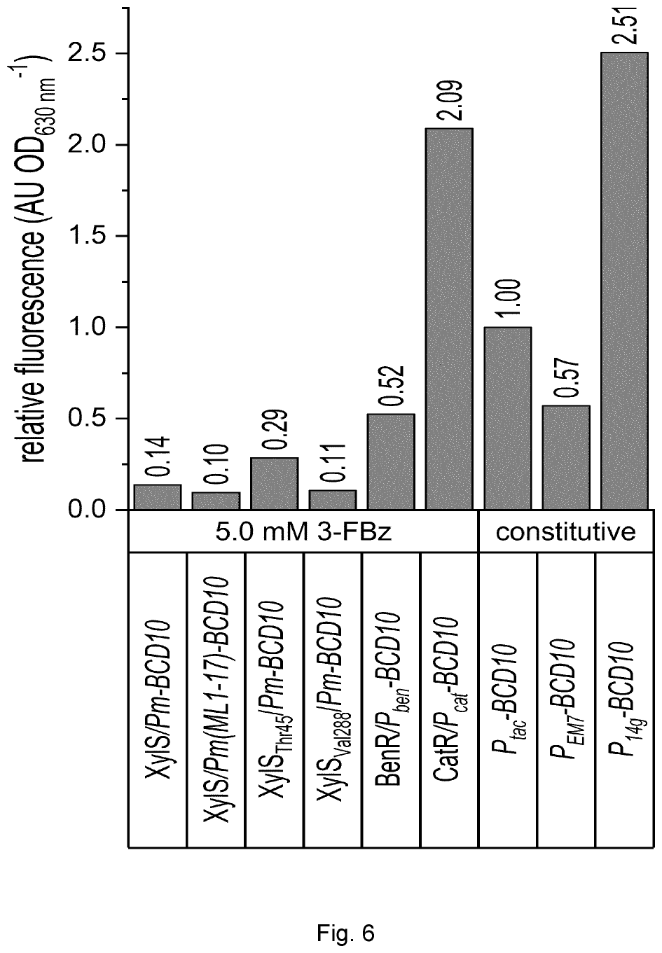

P_{14b} has a transcriptional strength that is 25% that of the strongest promoter reported for P. putida: P_{14g} (characterized in FIG. 6). Other medium-to-weak constitutive promoters may also be useful for the present invention. Thus, in some embodiments, the medium-to-weak constitutive promoter is J23108 (SEQ ID NO: 46) or a variant thereof having at least 80%, such as at least 85%, such as at least 90%, such as at least 95%, such as at least 99% homology or identity thereto. In some embodiments, the medium-to-weak constitutive promoter is P_{14d} (SEQ ID NO: 55) or a variant thereof having at least 80%, such as at least 85%, such as at least 90%, such as at least 95%, such as at least 99% homology or identity thereto. In some embodiments, the medium-to-weak constitutive promoter is J23114 (SEQ ID NO: 47) or a variant thereof having at least 80%, such as at least 85%, such as at least 90%, such as at least 95%, such as at least 99% homology or identity thereto. In some embodiments, the medium-to-weak constitutive promoter is JE1611 (SEQ ID NO: 65) or a variant thereof having at least 80%, such as at least 85%, such as at least 90%, such as at least 95%, such as at least 99% homology or identity thereto. In some embodiments, the medium-to-weak constitutive promoter is JEa2 (SEQ ID NO: 66) or a variant thereof having at least 80%, such as at least 85%, such as at least 90%, such as at least 95%, such as at least 99% homology or identity thereto. In some embodiments, the medium-to-weak constitutive promoter is J1312 (SEQ ID NO: 67) or a variant thereof having at least 80%, such as at least 85%, such as at least 90%, such as at least 95%, such as at least 99% homology or identity thereto.

The first and the second promoters may be different promoters. In some embodiments, the first and the second promoters are the same promoter. Thus, the first and the second promoters may be the same medium-to-weak constitutive promoter, such as P_{14b} (SEQ ID NO: 49) or a variant thereof having at least 80% homology or identity thereto.

In some embodiments, the first and/or the second strong translation initiation sequence is BCD2 (SEQ ID NO: 22) or a variant thereof having at least 80%, such as at least 85%, such as at least 90%, such as at least 95%, such as at least 99% homology or identity thereto. In some embodiments, the first and/or the second strong translation initiation sequence is BCD10 (SEQ ID NO: 16) or a variant thereof having at least 80%, such as at least 85%, such as at least 90%, such as at least 95%, such as at least 99% homology or identity thereto.

Any suitable nucleic acid sequence may be used to replace the translation initiation region of the gene encoding the first and/or the second catechol 1,2-dioxygenase. In some embodiments, the first and/or the second strong translation initiation sequence has similar translation initiation strength as BCD2 (SEQ ID NO: 22) or BCD10 (SEQ ID NO: 16). In some embodiments, the first and/or the second strong translation initiation sequence has a predicted translation rate of 3,000 to 80,000, predicted using the RBS Calculator available online at https://salislab.net/software/design_rbs_calculator.

In some embodiments, the first and/or the second strong translation initiation sequence is BCD1 (SEQ ID NO: 23) or a variant thereof having at least 80%, such as at least 85%, such as at least 90%, such as at least 95%, such as at least 99% homology or identity thereto. In some embodiments, the first and/or the second strong translation initiation sequence is BCD5 (SEQ ID NO: 78) or a variant thereof having at least 80%, such as at least 85%, such as at least 90%, such as at least 95%, such as at least 99% homology or identity thereto. In some embodiments, the first and/or the second strong translation initiation sequence is BCD6 (SEQ ID NO: 17) or a variant thereof having at least 80%, such as at least 85%, such as at least 90%, such as at least 95%, such as at least 99% homology or identity thereto. In some embodiments, the first and/or the second strong translation initiation sequence is BCD7 (SEQ ID NO: 45) or a variant thereof having at least 80%, such as at least 85%, such as at least 90%, such as at least 95%, such as at least 99% homology or identity thereto. In some embodiments, the first and/or the second strong translation initiation sequence is BCD9 (SEQ ID NO: 79) or a variant thereof having at least 80%, such as at least 85%, such as at least 90%, such as at least 95%, such as at least 99% homology or identity thereto. In some embodiments, the first and/or the second strong translation initiation sequence is BCD11 (SEQ ID NO: 44) or a variant thereof having at least 80%, such as at least 85%, such as at least 90%, such as at least 95%, such as at least 99% homology or identity thereto. In some embodiments, the first and/or the second strong translation initiation sequence is BCD12 (SEQ ID NO: 80) or a variant thereof having at least 80%, such as at least 85%, such as at least 90%, such as at least 95%, such as at least 99% homology or identity thereto. In some embodiments, the first and/or the second strong translation initiation sequence is BCD13 (SEQ ID NO: 81) or a variant thereof having at least 80%, such as at least 85%, such as at least 90%, such as at least 95%, such as at least 99% homology or identity thereto. In some embodiments, the first and/or the second strong translation initiation sequence is BCD18 (SEQ ID NO: 83) or a variant thereof having at least 80%, such as at least 85%, such as at least 90%, such as at least 95%, such as at least 99% homology or identity thereto. In some embodiments, the first and/or the second strong translation initiation sequence is BCD19 (SEQ ID NO: 82) or a variant thereof having at least 80%, such as at least 85%, such as at least 90%, such as at least 95%, such as at least 99% homology or identity thereto. In some embodiments, the first and/or the second strong translation initiation sequence is BCD20 (SEQ ID NO: 84) or a variant thereof having at least 80%, such as at least 85%, such as at least 90%, such as at least 95%, such as at least 99% homology or identity thereto.

The first and the second strong translational initiation sequences may be different strong translational initiation sequences. In some embodiments, the first and the second strong translational initiation sequences are the same strong translational initiation sequence.

In some embodiments, the expression of the first catechol 1,2-dioxygenase described herein above, for example CatA-I, is under the control of a first expression module comprising a medium-low constitutive promoter, said first expression module further comprising a first strong translational coupler, whereby said first catechol 1,2-dioxygenase is constitutively expressed, and the expression of the second catechol 1,2-dioxygenase, for example CatA-II, described herein above is under the control of a second expression module comprising a second constitutive medium-low promoter, said second expression module further comprising a second strong translational coupler whereby said second catechol 1,2-dioxygenase is constitutively expressed. In some embodiments the first medium-low constitutive promoter of said first expression module is P_{14b} (SEQ ID NO: 49) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, and the first strong translational coupler is BCD2 (SEQ ID NO: 22) or BCD10 (SEQ ID NO: 16), or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, preferably the first strong translational coupler is BCD10 (SEQ ID NO: 16) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments, the second medium-low constitutive promoter of said second expression module is P_{14b} (SEQ ID NO: 49) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, and the second strong translational coupler is BCD10 (SEQ ID NO: 16) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the first and/or the second promoter is a first and/or a second inducible promoter, optionally wherein the first and/or second promoter is coupled with a sequence encoding an associated activator protein.

In some embodiments, the first and/or the second inducible promoter is the Pm variant ML1-17 (SEQ ID NO: 18) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments, the first and/or the second inducible promoter is Pm (SEQ ID NO: 15) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the associated activator protein is XylS (SEQ ID NO: 21) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

Other useful inducible promoter systems that mediate a similar transcription level at a certain inducer compound concentration includes the LacI^{q}/P_{trc}, CprK1/PDB3, ChnR/P_{chnB}, and RhaS-RhaR/Pᵣₕₐ systems.

In some embodiments, the expression of the first catechol 1,2-dioxygenase described herein above is under the control of a first expression module comprising a first inducible promoter coupled with a sequence encoding an associated activator protein, said first expression module further comprising a first strong translational coupler, whereby said first catechol 1,2-dioxygenase is expressed strongly in response to 3-fluorobenzoate, and the expression of the second catechol 1,2-dioxygenase described herein above is under the control of a second expression module comprising a second inducible promoter, said second expression module further comprising a second strong translational coupler whereby said second catechol 1,2-dioxygenase is expressed strongly in response to 3-fluorobenzoate. In some embodiments the first inducible promoter of said first expression module is the Pm variant ML1-17 (SEQ ID NO: 18), the associated activator protein is XylS (SEQ ID NO: 21), and the first strong translational coupler is BCD2 (SEQ ID NO: 22) or BCD10 (SEQ ID NO: 16), or variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, preferably the first strong translational coupler is BCD10 (SEQ ID NO: 16) or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments the first strong translational coupler is BCD11 (SEQ ID NO: 44), BCD7 (SEQ ID NO: 45), BCD6 (SEQ ID NO: 17), BCD5 (SEQ ID NO: 19), or BCD1 (SEQ ID NO: 23) or variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments, the second inducible promoter of said second expression module is Pm (SEQ ID NO: 15), and the second strong translational coupler is BCD10 (SEQ ID NO: 16), or variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the bacterial cell expresses the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenase CatA-I (SEQ ID NO: 9), or functional variants thereof as described herein.

In some embodiments, the bacterial cell expresses the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenases CatA-I (SEQ ID NO: 9) and CatA-II (SEQ ID NO: 11), or functional variants thereof as described herein.

In some embodiments, the bacterial cell expresses the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenase CatA-I (SEQ ID NO: 9), wherein BenABC is expressed under the control of the medium-strong constitutive promoter P_{tac} (SEQ ID NO: 15) and CatA-I is expressed under the control of the first expression module comprising or consisting of the medium-to-weak constitutive promoter P_{14b} (SEQ ID NO: 49), and the first strong translational coupler BCD2 (SEQ ID NO: 22) or BCD10 (SEQ ID NO: 16), or functional variants thereof as described herein.

In some embodiments, the bacterial cell expresses the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenase CatA-I (SEQ ID NO: 9), wherein BenABC is expressed under the control of the medium-strong constitutive promoter P_{tac} (SEQ ID NO: 15) and CatA-I is expressed under the control of the first expression module comprising or consisting of the first inducible promoter Pm variant ML1-17 (SEQ ID NO: 18), the associated activator protein is XylS (SEQ ID NO: 21), and the first strong translational coupler BCD2 (SEQ ID NO: 22) or BCD10 (SEQ ID NO: 16), or functional variants thereof as described herein.

In some embodiments, the bacterial cell expresses the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenases CatA-I (SEQ ID NO: 9) and CatA-II (SEQ ID NO: 11), wherein BenABC is expressed under the control of the medium-strong constitutive promoter P_{tac} (SEQ ID NO: 15) and CatA-I is expressed under the control of the first expression module comprising or consisting of the medium-to-weak constitutive promoter P_{14b} (SEQ ID NO: 49), and the first strong translational coupler BCD2 (SEQ ID NO: 22) or BCD10 (SEQ ID NO: 16), and wherein CatA-II is expressed under the control of the second expression module comprising or consisting of the medium-to-weak constitutive promoter P_{14b} (SEQ ID NO: 49) and the second strong translational coupler BCD10 (SEQ ID NO: 16), or functional variants thereof as described herein.

In some embodiments, the bacterial cell expresses the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenases CatA-I (SEQ ID NO: 9) and CatA-II (SEQ ID NO: 11), wherein BenABC is expressed under the control of the medium-strong constitutive promoter P_{tac} (SEQ ID NO: 15) and CatA-I is expressed under the control of the first expression module comprising or consisting of the first inducible promoter Pm variant ML1-17 (SEQ ID NO: 18), the associated activator protein XylS (SEQ ID NO: 21), and the first strong translational coupler BCD2 (SEQ ID NO: 22) or BCD10 (SEQ ID NO: 16), and wherein CatA-II is expressed under the control of the second expression module comprising or consisting of the inducible promoter Pm (SEQ ID NO: 15) and the second strong translational coupler BCD10 (SEQ ID NO: 16), or functional variants thereof as described herein.

### Titers

In some embodiments, the bacterial cell is capable of producing 2-fluoro-cis,cis-muconate with a titer of at least 500 mg/L, such as at least 750 mg/L, such as at least 1 g/L, such as at least 1.5 g/L, such as at least 2 g/L, such as at least 2.5 g/L, such as at least 3 g/L, such as at least 3.5 g/L, such as at least 4 g/L, such as at least 4.5 g/L, such as at least 5 g/L, such as at least 6 g/L, such as at least 7 g/L, such as at least 8 g/L, such as at least 9 g/L, such as at least 10 g/L, such as at least 15 g/L, such as at least 20 g/L, such as at least 25 g/L, such as at least 30 g/L, such as at least 40 g/L, such as at least 50 g/L, such as at least 60 g/L, such as at least 80 g/L, such as at least 100 g/L, such as at least 150 g/L, such as at least 200 g/L, such as at least 250 g/L, such as at least 300 g/L or more.

### Other Modifications

As sugars may comprise the main carbon source for the bacterial cell, and without being bound by theory, modifications in glucose metabolism to increase growth performance on sugars and to avoid by-product formation typically associated with glucose utilization, in order to allow a faster growth rate of the cell may further increase the titers of 2-fluoro-cis,cis-muconate.

In some embodiments, the bacterial cell is as described above and further comprises modifications resulting in increased growth performance on sugars. In some embodiments, the bacterial cell further comprises deletion of the global catabolite repression control protein Crc (SEQ ID NO: 36) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the bacterial cell further comprises deletion of the global catabolite repression control protein Crc encoded by SEQ ID NO: 37 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

Without being bound by theory, two predominant by-products of glucose consumption in Pseudomonas putida are gluconate and 2-ketogluconate, due to the constitutively expressed, periplasmic glucose dehydrogenase (Gcd), and gluconate dehydrogenase (Gad). With fast-growing wildtype-cells, those two compounds are usually co-consumed together with glucose and result in no adverse effects on the growth rate. However, under growth-restricting conditions such as in the presence of growth-inhibiting agents (e.g. fluorinated compounds), or with cells showing slower growth rates due to genomic modifications, the production of gluconate and 2-ketogluconate exceeds their consumption, which can cause a decrease in pH, which may adversely affect growth rates. It may thus be beneficial to decrease the formation of gluconate and 2-ketogluconate to further increase the titers of 2-fluoro-cis,cis-muconate.

In some embodiments, the bacterial cell further comprises modifications to decrease by-product formation typically associated with glucose utilization. In some embodiments, the bacterial cell further comprises a mutation resulting in partial or total loss of activity of at least one of the proteins selected from the group consisting of the gamma subunit of Gad (SEQ ID NO: 28), the flavoprotein subunit of Gad (SEQ ID NO: 30), the cytochrome c subunit of Gad (SEQ ID NO: 32), and Gcd (SEQ ID NO: 34) or functional variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the bacterial cell further comprises a mutation resulting in partial or total loss of activity of the gamma subunit of Gad encoded by SEQ ID NO: 29 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments, the bacterial cell further comprises a mutation resulting in partial or total loss of activity of the flavoprotein subunit of Gad encoded by SEQ ID NO: 31 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments, the bacterial cell further comprises a mutation resulting in partial or total loss of activity of the cytochrome c subunit of Gad encoded by SEQ ID NO: 33 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments, the bacterial cell further comprises a mutation resulting in partial or total loss of activity of Gcd encoded by SEQ ID NO: 35 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

Because the present pathways require 3-fluorobenzoate as a first substrate, and without being bound by theory, it may be advantageous to modify the bacterial cell in such a manner that the uptake of 3-fluorobenzoate into the cell is increased, so that more 3-fluorobenzoate is available for conversion into 2-fluoro-cis,cis-muconate, thereby further increasing the titers of 2-fluoro-cis,cis-muconate.

In some embodiments, the bacterial cell further comprises modifications resulting in overexpression of genes encoding transporter proteins involved in 3-fluorobenzoate uptake. In some embodiments, the bacterial cell further comprises modifications resulting in overexpression one of the proteins selected from the group consisting of BenK (SEQ ID NO: 38), BenE-II (SEQ ID NO: 40), and NicP-I (SEQ ID NO: 42) or functional variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the bacterial cell further comprises modifications resulting in overexpression of BenK encoded by SEQ ID NO: 39 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments, the bacterial cell further comprises modifications resulting in overexpression of BenE-II encoded by SEQ ID NO: 41 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. In some embodiments, the bacterial cell further comprises modifications resulting in overexpression of NicP-1 encoded by SEQ ID NO: 43 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

As the present bioconversion pathway comprises several oxygen-consuming reactions, and without being bound by theory, increasing intracellular oxygen availability may further increase flux through the pathway, and thus further increase the titers of 2-fluoro-cis,cis-muconate.

In some embodiments, the bacterial cell further expresses a bacterial haemoglobin, whereby intracellular oxygen availability is increased. In some embodiments, the bacterial haemoglobin is a heterologous bacterial hemoglobin (EC: 1.14.12.17). In some embodiments, the heterologous bacterial hemoglobin is VHb (SEQ ID NO: 26) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, to increase oxygen availability for the oxygen-consuming reactions of the bioconversion pathway.

In some embodiments, the bacterial cell further expresses the heterologous bacterial haemoglobin VHb encoded by SEQ ID NO: 27 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

The bacterial cell may be as described herein.

### Expression of Native or Heterologous Enzymes

In some embodiments, one or more of the genes encoding the benzoate 1,2-dioxygenase, the medium-strong constitutive promoter, the benzoate-1,2-dihydrodiol dehydrogenase, the first catechol 1,2-dioxygenase, the second catechol 1,2-dioxygenase, the first expression module and/or the second expression module are codon-optimized for the bacterial cell.

In some embodiments, one or more of the genes encoding the benzoate 1,2-dioxygenase, the medium-strong constitutive promoter, the benzoate-1,2-dihydrodiol dehydrogenase, the first catechol 1,2-dioxygenase, the second catechol 1,2-dioxygenase, the first expression module and/or the second expression module are present in multiple copy number.

In some embodiments, one or more of the genes encoding the benzoate 1,2-dioxygenase, the medium-strong constitutive promoter, the benzoate-1,2-dihydrodiol dehydrogenase, the first catechol 1,2-dioxygenase, the second catechol 1,2-dioxygenase, the first expression module and/or the second expression module are integrated in the genome of the bacterial cell.

In some embodiments, one or more of the genes encoding the benzoate 1,2-dioxygenase, the medium-strong constitutive promoter, the benzoate-1,2-dihydrodiol dehydrogenase, the first catechol 1,2-dioxygenase, the second catechol 1,2-dioxygenase, the first expression module and/or the second expression module are expressed from a vector such as a plasmid.

### Methods of Producing 2-Fluoro-Cis,Cis-Muconic Acid and Derivatives Thereof

The present disclosure relates to methods for producing 2-fluoro-cis,cis-muconic acid and derivatives thereof. The bacterial cells and nucleic acid constructs described herein are useful for bacterial cell-based production of 2-fluoro-cis,cis-muconic acid and derivatives thereof, including 2-fluoroadipic acid, [—OC—(F)—(CH₂)₄—CO—NH—(CH₂)₆—NH—], (a fluorinated version of the polyamide Nylon 6-6), 2-fluoro-1,6-hexanediol, fluoro-terephthalic acid, fluoro-trimellitic acid, fluorinated polyesters, fluorinated polyurethanes, fluorinated acrylics, fluorinated adhesives and fluorinated dyes.

Herein is provided a method of producing 2-fluoro-cis,cis-muconate, and optionally derivatives thereof, in a bacterial cell, said method comprising the steps of providing a bacterial cell and incubating said bacterial cell in a medium in the presence of 3-fluorobenzoate or a salt thereof, said bacterial cell expressing:
a native or heterologous benzoate 1,2-dioxygenase (EC: 1.14.12.10);
a native or heterologous benzoate-1,2-dihydrodiol dehydrogenase (EC: 1.3.1.25);
and
a first, and optionally a second, native or heterologous catechol 1,2-dioxygenase (EC: 1.13.11.1),
   wherein:
the expression of said benzoate 1,2-dioxygenase is under the control of a medium-strong constitutive promoter;
the expression of said first catechol 1,2-dioxygenase is under the control of a first expression module comprising a first promoter, said first expression module further comprising a first strong translation initiation sequence such as a first strong translational coupler; and
the expression of said second catechol 1,2-dioxygenase is under the control of a second expression module comprising a second promoter, said second expression module further comprising a second strong translational initiation sequence such as a second strong translational coupler.

Herein is provided a method of producing 2-fluoro-cis,cis-muconate, and optionally derivatives thereof, in a bacterial cell, said method comprising the steps of providing a bacterial cell and incubating said bacterial cell in a medium in the presence of 3-fluorobenzoate or a salt thereof, said bacterial cell expressing:
a native or heterologous benzoate 1,2-dioxygenase (EC: 1.14.12.10);
a native or heterologous benzoate-1,2-dihydrodiol dehydrogenase (EC: 1.3.1.25); and
a first native or heterologous catechol 1,2-dioxygenase (EC: 1.13.11.1),
   wherein:
the expression of said benzoate 1,2-dioxygenase is under the control of a medium-strong constitutive promoter;
the expression of said first catechol 1,2-dioxygenase is under the control of a first expression module comprising a first inducible promoter coupled with a sequence encoding an associated activator protein, said first expression module further comprising a first strong translational coupler.

In some embodiments, the present disclosure provides a method of producing 2-fluoro-cis,cis-muconate, and optionally derivatives thereof, in a bacterial cell, said method comprising the steps of providing a bacterial cell and incubating said bacterial cell in a medium in the presence of 3-fluorobenzoate or a salt thereof, said bacterial cell expressing:
a native or heterologous benzoate 1,2-dioxygenase (EC: 1.14.12.10);
a native or heterologous benzoate-1,2-dihydrodiol dehydrogenase (EC: 1.3.1.25); and
a first and a second native or heterologous catechol 1,2-dioxygenases (EC: 1.13.11.1),
   wherein:
the expression of said benzoate 1,2-dioxygenase is under the control of a medium-strong constitutive promoter;
the expression of said first catechol 1,2-dioxygenase is under the control of a first expression module comprising a first inducible promoter coupled with a sequence encoding an associated activator protein, said first expression module further comprising a first strong translational coupler; and
the expression of said second catechol 1,2-dioxygenase is under the control of a second expression module comprising a second inducible promoter, said second expression module further comprising a second strong translational coupler.

In some embodiments, the benzoate 1,2-dioxygenase (EC: 1.14.12.10) expressed by the bacterial cell is a native or heterologous benzoate 1,2-dioxygenase, as described herein above in the section “Bacterial cell”, and may further comprise any of the modifications described in “Other modifications”. In particular, the bacterial cell may express BenABC or a functional variant thereof having at least 80% homology or identity thereto, as detailed above. In particular, the gene(s) encoding the benzoate 1,2-dioxygenase may be under the control of a specific promoter, such as P_{tac} or a variant thereof having at least 80% homology or identity thereto, as described herein above in the section “Bacterial cell”.

In some embodiments, the benzoate-1,2-dihydrodiol dehydrogenase (EC: 1.3.1.25) expressed by the bacterial cell is a native or heterologous benzoate-1,2-dihydrodiol dehydrogenase, as described herein above in the section “Bacterial cell”, and may further comprise any of the modifications described in “Other modifications”. In particular, the bacterial cell may express BenD or a functional variant thereof having at least 80% homology or identity thereto, as detailed above.

In some embodiments, the first and/or second catechol 1,2-dioxygenase (EC: 1.13.11.1) expressed by the bacterial cell is a native or heterologous catechol 1,2-dioxygenase, as described herein above in the section “Bacterial cell”, and may further comprise any of the modifications described in “Other modifications”. In particular, the bacterial cell may express CatA-I and/or CatA-II as detailed above. In particular, the expression of the gene(s) encoding the benzoate 1,2-dioxygenase(s) may be under the control of specific first and/or second expression module(s) as described herein above in the section “Bacterial cell”. In particular, as described herein above, the first expression module may comprise the P_{14b} promoter or a variant thereof having at least 80% homology or identity thereto, and the strong translational coupler BCD2 or BCD10 or a variant thereof having at least 80% homology or identity thereto. In particular, as described herein above, the second expression module may comprise the P_{14b} promoter or a variant thereof having at least 80% homology or identity thereto and the strong translational coupler BCD10 or a variant thereof having at least 80% homology or identity thereto. In particular, as described herein above, the first expression module may comprise the Pm promoter variant ML1-17 or a variant thereof having at least 80% homology or identity thereto, the associated activator protein XylS or a functional variant thereof having at least 80% homology or identity thereto, and the strong translational coupler BCD2 or BCD10 or a variant thereof having at least 80% homology or identity thereto. In particular, as described herein above, the second expression module may comprise the Pm promoter or a variant thereof having at least 80% homology or identity thereto and the strong translational coupler BCD10 or a variant thereof having at least 80% homology or identity thereto.

The present method requires the precursor molecule 3-fluorobenzoate or a salt thereof. 3-fluorobenzoate or a salt thereof may be provided directly in the medium or it may be derived from a provided precursor molecule through a metabolic process by the bacterial cell.

In some embodiments, the concentration of 3-fluorobenzoate or a salt thereof in said medium is at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM such as at least 25 mM, such as 30 mM or more. In some embodiments, the concentration of 3-fluorobenzoate or a salt thereof in said medium is at least 35 mM, such as at least 40 mM, such as at least 45 mM, such as 50 mM or more. In some embodiments, the concentration of 3-fluorobenzoate or a salt thereof in said medium is between 5 mM and 15 mM. In some specific embodiments, the concentration of 3-fluorobenzoate or a salt thereof in said medium is 10 mM. In some embodiments, the concentration of 3-fluorobenzoate or a salt thereof in said medium is between 10 mM and 50 mM. In some specific embodiments, the concentration of 3-fluorobenzoate or a salt thereof in said medium is 20 mM. In some specific embodiments, the concentration of 3-fluorobenzoate or a salt thereof in said medium is 30 mM. In some specific embodiments, the concentration of 3-fluorobenzoate or a salt thereof in said medium is 40 mM. In some specific embodiments, the concentration of 3-fluorobenzoate or a salt thereof in said medium is 50 mM.

In some embodiments, the method is for production of 2-fluoroadipic acid, and further comprises a step of converting the 2-fluoro-cis,cis-muconate to 2-fluoroadipic acid. In some embodiments, the method is for production of [—OC—(F)—(CH₂)₄—CO—NH—(CH₂)₆—NH—]ₙ, and further comprises a step of converting the 2-fluoro-cis,cis-muconate to [—OC—(F)—(CH₂)₄—CO—NH—(CH₂)₆—NH—]ₙ. In some embodiments, the method is for production of 2-fluoro-1,6-hexanediol, and further comprises a step of converting the 2-fluoro-cis,cis-muconate to 2-fluoro-1,6-hexanediol. In some embodiments, the method is for production of fluoro-terephthalic acid, and further comprises a step of converting the 2-fluoro-cis,cis-muconate to fluoro-terephthalic acid. In some embodiments, the method is for production of fluoro-trimellitic acid, and further comprises a step of converting the 2-fluoro-cis,cis-muconate to fluoro-trimellitic acid. In some embodiments, the method is for production of a fluorinated polyester, and further comprises a step of converting the 2-fluoro-cis,cis-muconate to a fluorinated polyester. In some embodiments, the method is for production of a fluorinated polyurethane, and further comprises a step of converting the 2-fluoro-cis,cis-muconate to a fluorinated polyurethane. In some embodiments, the method is for production of a fluorinated acrylic, and further comprises a step of converting the 2-fluoro-cis,cis-muconate to a fluorinated acrylic. In some embodiments, the method is for production of a fluorinated adhesive, and further comprises a step of converting the 2-fluoro-cis,cis-muconate to a fluorinated adhesive. In some embodiments, the method is for production of a fluorinated dye, and further comprises a step of converting the 2-fluoro-cis,cis-muconate to a fluorinated dye.

### Recovering the 2-Fluoro-Cis,Cis-Muconate

The present methods may comprise a further step of recovering the 2-fluoro-cis,cis-muconate obtained by the methods disclosed herein. Methods for recovering the products obtained by the present disclosure are known in the art, for example organic solvent extraction followed by lyophilisation and purification by preparative HPLC or similar column purification techniques. Another example includes pH/temperature shift crystallization (acidification and cooling), as described by Vardon, Rorrer et al. (2016).

The bacterial cell is preferably as defined herein.

### Titers

The present methods are useful for producing 2-fluoro-cis,cis-muconate with high titers.

In some embodiments, 2-fluoro-cis,cis-muconate is produced with a titer of at least 500 mg/L, such as at least 750 mg/L, such as at least 1 g/L, such as at least 1.5 g/L, such as at least 2 g/L, such as at least 2.5 g/L, such as at least 3 g/L, such as at least 3.5 g/L, such as at least 4 g/L, such as at least 4.5 g/L, such as at least 5 g/L, such as at least 6 g/L, such as at least 7 g/L, such as at least 8 g/L, such as at least 9 g/L, such as at least 10 g/L, such as at least 15 g/L, such as at least 20 g/L, such as at least 25 g/L, such as at least 30 g/L, such as at least 40 g/L, such as at least 50 g/L, such as at least 60 g/L, such as at least 80 g/L, such as at least 100 g/L, such as at least 150 g/L, such as at least 200 g/L, such as at least 250 g/L, such as at least 300 g/L or more.

The accumulation of 2-fluoro-cis,cis-muconate in the growth medium may be toxic to the bacterial cell, and may thus impair cell growth. In some embodiments, the tolerance of the bacterial cell to 2-fluoro-cis,cis-muconate is further improved via adaptive laboratory evolution. In preferred embodiments, the bacterial cell is a Pseudomonas putida cell.

### 2-Fluoro-Cis,Cis-Muconate or a Derivative Thereof

Herein is provided 2-fluoro-cis,cis-muconic acid and derivatives thereof.

In some embodiments, the present disclosure provides 2-fluoro-cis,cis-muconate.

In some embodiments, the present disclosure provides 2-fluoroadipic acid. 2-fluoroadipic acid may be obtained through chemical conversion of 2-fluoro-cis,cis-muconate to 2-fluoroadipic acid.

In some embodiments, the present disclosure provides [—OC—(F)—(CH₂)₄—CO—NH—(CH₂)₆—NH—]ₙ (a fluorinated version of the polyamide Nylon 6-6). [—OC—(F)—(CH₂)₄—CO—NH—(CH₂)₆—NH—]ₙ may be obtained through chemical conversion of 2-fluoro-cis,cis-muconate to [—OC—(F)—(CH₂)₄—CO—NH—(CH₂)₆—NH—]ₙ, such as by condensing the 2-fluoro-cis,cis-muconate derivative 2-fluoroadipic acid with hexamethylenediamine to form [—OC—(F)—(CH₂)₄—CO—NH—(CH₂)₆—NH—]ₙ.

In some embodiments, the present disclosure provides 2-fluoro-1,6-hexanediol. 2-fluoro-1,6-hexanediol may be obtained through chemical conversion of 2-fluoro-cis,cis-muconate to 2-fluoro-1,6-hexanediol. 2-fluoro-1,6-hexanediol may be used for fluorinated polyester and polyurethane production, and as an intermediate in the production of fluorinated acrylics, adhesives and dyes.

In some embodiments, the present disclosure provides fluoro-terephthalic acid. Fluoro-terephthalic acid may be obtained through chemical conversion of 2-fluoro-cis,cis-muconate to fluoro-terephthalic acid.

In some embodiments, the present disclosure provides fluoro-trimellitic acid. Fluoro-trimellitic acid may be obtained through chemical conversion of 2-fluoro-cis,cis-muconate to fluoro-trimellitic acid.

In some embodiments, the present disclosure provides a fluorinated polyester.

Fluorinated polyesters may be obtained through chemical conversion of 2-fluoro-cis,cis-muconate to a fluorinated polyester.

In some embodiments, the present disclosure provides a fluorinated polyurethane.

Fluorinated polyurethanes may be obtained through chemical conversion of 2-fluoro-cis,cis-muconate to a fluorinated polyurethane.

In some embodiments, the present disclosure provides a fluorinated acrylic.

Fluorinated acrylics may be obtained through chemical conversion of 2-fluoro-cis,cis-muconate to a fluorinated acrylic.

In some embodiments, the present disclosure provides a fluorinated adhesive.

Fluorinated adhesives may be obtained through chemical conversion of 2-fluoro-cis,cis-muconate to a fluorinated adhesive.

In some embodiments, the present disclosure provides a fluorinated dye. Fluorinated dyes may be obtained through chemical conversion of 2-fluoro-cis,cis-muconate to a fluorinated dye.

In some embodiments, the present disclosure provides 2-fluoro-cis,cis-muconic acid and derivatives thereof and derivatives thereof obtainable by a method as disclosed herein.

In some embodiments, the present disclosure provides 2-fluoro-cis,cis-muconate. The thus obtained 2-fluoro-cis,cis-muconate can be further converted into other compounds or derivatives, as detailed herein. The methods disclosed herein may thus further comprise a step of converting the 2-fluoro-cis,cis-muconate to another compound or derivative.

In some embodiments, the present disclosure provides 2-fluoro-cis,cis-muconate which is further converted to 2-fluoroadipic acid. 2-fluoroadipic acid may be obtained through conversion, for example chemical conversion of 2-fluoro-cis,cis-muconate to 2-fluoroadipic acid. Accordingly, in some embodiments the method further comprises a step of conversion of the 2-fluoro-cis,cis-muconate which is further converted to 2-fluoroadipic acid.

In some embodiments, the present disclosure provides 2-fluoro-cis,cis-muconate which is further converted to [—OC—(F)—(CH₂)₄—CO—NH—(CH₂)₆—NH—]ₙ (a fluorinated version of the polyamide Nylon 6-6). [—OC—(F)—(CH₂)₄—CO—NH—(CH₂)₆—NH—]ₙ may be obtained through conversion, for example chemical conversion of 2-fluoro-cis,cis-muconate to [—OC—(F)—(CH₂)₄—CO—NH—(CH₂)₆—NH—]ₙ, such as by condensing the 2-fluoro-cis,cis-muconate derivative 2-fluoroadipic acid with hexamethylenediamine to form [—OC—(F)—(CH₂)₄—CO—NH—(CH₂)₆—NH—]ₙ. Accordingly, in some embodiments the method further comprises a step of conversion of the 2-fluoro-cis,cis-muconate which is further converted to [—OC—(F)—(CH₂)₄—CO—NH—(CH₂)₆—NH—]ₙ.

In some embodiments, the present disclosure provides 2-fluoro-cis,cis-muconate which is further converted to 2-fluoro-1,6-hexanediol. 2-fluoro-1,6-hexanediol may be obtained through conversion, for example chemical conversion of 2-fluoro-cis,cis-muconate to 2-fluoro-1,6-hexanediol. 2-fluoro-1,6-hexanediol may be used for fluorinated polyester and polyurethane production, and as an intermediate in the production of fluorinated acrylics, adhesives and dyes. Accordingly, in some embodiments the method further comprises a step of conversion of the 2-fluoro-cis,cis-muconate which is further converted to 2-fluoro-1,6-hexanediol.

In some embodiments, the present disclosure provides 2-fluoro-cis,cis-muconate which is further converted to fluoro-terephthalic acid. Fluoro-terephthalic acid may be obtained through conversion, for example chemical conversion of 2-fluoro-cis,cis-muconate to fluoro-terephthalic acid. Accordingly, in some embodiments the method further comprises a step of conversion of the 2-fluoro-cis,cis-muconate which is further converted to fluoro-terephthalic acid.

In some embodiments, the present disclosure provides 2-fluoro-cis,cis-muconate which is further converted to fluoro-trimellitic acid. Fluoro-trimellitic acid may be obtained through conversion, for example chemical conversion of 2-fluoro-cis,cis-muconate to fluoro-trimellitic acid. Accordingly, in some embodiments the method further comprises a step of conversion of the 2-fluoro-cis,cis-muconate which is further converted to fluoro-trimellitic acid.

In some embodiments, the present disclosure provides 2-fluoro-cis,cis-muconate which is further converted to a fluorinated polyester. Fluorinated polyesters may be obtained through conversion, for example chemical conversion of 2-fluoro-cis,cis-muconate to a fluorinated polyester. Accordingly, in some embodiments the method further comprises a step of conversion of the 2-fluoro-cis,cis-muconate which is further converted to a fluorinated polyester.

In some embodiments, the present disclosure provides 2-fluoro-cis,cis-muconate which is further converted to a fluorinated polyurethane. Fluorinated polyurethanes may be obtained through conversion, for example chemical conversion of 2-fluoro-cis,cis-muconate to a fluorinated polyurethane. Accordingly, in some embodiments the method further comprises a step of conversion of the 2-fluoro-cis,cis-muconate which is further converted to a fluorinated polyurethane.

In some embodiments, the present disclosure provides 2-fluoro-cis,cis-muconate which is further converted to a fluorinated acrylic. Fluorinated acrylics may be obtained through conversion, for example chemical conversion of 2-fluoro-cis,cis-muconate to a fluorinated acrylic. Accordingly, in some embodiments the method further comprises a step of conversion of the 2-fluoro-cis,cis-muconate which is further converted to a fluorinated acrylic.

In some embodiments, the present disclosure provides 2-fluoro-cis,cis-muconate which is further converted to a fluorinated adhesive. Fluorinated adhesives may be obtained through conversion, for example chemical conversion of 2-fluoro-cis,cis-muconate to a fluorinated adhesive. Accordingly, in some embodiments the method further comprises a step of conversion of the 2-fluoro-cis,cis-muconate which is further converted to a fluorinated adhesive.

In some embodiments, the present disclosure provides 2-fluoro-cis,cis-muconate which is further converted to a fluorinated dye. Fluorinated dyes may be obtained through conversion, for example chemical conversion of 2-fluoro-cis,cis-muconate to a fluorinated dye. Accordingly, in some embodiments the method further comprises a step of conversion of the 2-fluoro-cis,cis-muconate which is further converted to a fluorinated dye.

In some embodiments, the enantiomeric purity of the 2-fluoro-cis,cis-muconate may be at least 60%, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100%.

### Nucleic Acid Constructs

Also provided herein are nucleic acid constructs useful for engineering a bacterial cell capable of producing 2-fluoro-cis,cis-muconate or derivatives thereof as described above. The present nucleic acid constructs may be provided as one or more nucleic acid molecules or polynucleotides, for example they may be comprised in one or more vectors. Such nucleic acids may be introduced in the bacterial cell by methods known in the art.

It will be understood that throughout the present disclosure, the term ‘nucleic acid encoding an activity’ shall refer to a nucleic acid molecule capable of encoding a peptide, a protein or a fragment thereof having said activity. Such nucleic acid molecules may be open reading frames or genes, or fragments thereof.

Herein is provided a nucleic acid construct for modifying a cell comprising:
a first polynucleotide encoding a native or heterologous benzoate 1,2-dioxygenase (EC: 1.14.12.10) and further comprising a medium-strong constitutive promoter;
a second polynucleotide encoding a native or heterologous benzoate-1,2-dihydrodiol dehydrogenase (EC: 1.3.1.25); and
a third polynucleotide encoding a first native or heterologous catechol 1,2-dioxygenase (EC: 1.13.11.1), a first expression module comprising a first promoter, said first expression module further comprising a first strong translation initiation sequence such as a first strong translational coupler; and
optionally, a fourth polynucleotide encoding a second native or heterologous catechol 1,2-dioxygenase (EC: 1.13.11.1), a second expression module comprising a second promoter, said second expression module further comprising a second strong translational initiation sequence such as a second strong translational coupler.

Herein is provided a nucleic acid construct for modifying a cell comprising:
a first polynucleotide encoding a native or heterologous benzoate 1,2-dioxygenase (EC: 1.14.12.10) and further comprising a medium-strong constitutive promoter;
a second polynucleotide encoding a native or heterologous benzoate-1,2-dihydrodiol dehydrogenase (EC: 1.3.1.25); and
a third polynucleotide encoding a first native or heterologous catechol 1,2-dioxygenase (EC: 1.13.11.1), a first expression module comprising a first inducible promoter and a sequence encoding an associated activator protein, said first expression module further comprising a first strong translational coupler.

Herein is also provided a nucleic acid construct for modifying a cell comprising:
a first polynucleotide encoding a native or heterologous benzoate 1,2-dioxygenase (EC: 1.14.12.10) and further comprising a medium-strong constitutive promoter;
a second polynucleotide encoding a native or heterologous benzoate-1,2-dihydrodiol dehydrogenase (EC: 1.3.1.25);
a third polynucleotide encoding a first native or heterologous catechol 1,2-dioxygenase (EC: 1.13.11.1), a first expression module comprising a first inducible promoter and a sequence encoding an associated activator protein, said first expression module further comprising a first strong translational coupler; and
a fourth polynucleotide encoding a second native or heterologous catechol 1,2-dioxygenase (EC: 1.13.11.1), a second expression module comprising a second inducible promoter, said second expression module further comprising a second strong translational coupler.

The polynucleotide encoding the benzoate 1,2-dioxygenase may herein be referred to as the “first polynucleotide”. The polynucleotide encoding the benzoate-1,2-dihydrodiol dehydrogenase may be referred to as the “second polynucleotide”. The polynucleotide encoding the first catechol 1,2-dioxygenase may be referred to as the “third polynucleotide”. The polynucleotide encoding the second catechol 1,2-dioxygenase may be referred to as the “fourth polynucleotide”. This does not imply that the construct comprises four polynucleotides in total; in some embodiments the cell comprises only the first, second and third polynucleotides.

In some embodiments, the benzoate 1,2-dioxygenase (EC: 1.14.12.10) is a native or heterologous benzoate 1,2-dioxygenase, as described herein above in the section “Bacterial cell”, and may further comprise any of the modifications described in “Other modifications”. In particular, the benzoate 1,2-dioxygenase may be BenABC or a functional variant thereof having at least 80% homology or identity thereto, the expression of which may be regulated as detailed above. In particular, the gene(s) encoding the benzoate 1,2-dioxygenase may be under the control of a specific promoter, such as P_{tac} or a variant thereof having at least 80% homology or identity thereto, as described herein above in the section “Bacterial cell”.

In some embodiments, the benzoate-1,2-dihydrodiol dehydrogenase (EC: 1.3.1.25) is a native or heterologous benzoate-1,2-dihydrodiol dehydrogenase, as described herein above in the section “Bacterial cell”, and may further comprise any of the modifications described in “Other modifications”. In particular, the benzoate-1,2-dihydrodiol dehydrogenase may be BenD or a functional variant thereof having at least 80% homology or identity thereto, the expression of which may be regulated as detailed above.

In some embodiments, the first and/or second catechol 1,2-dioxygenase (EC: 1.13.11.1) is a native or heterologous catechol 1,2-dioxygenase, as described herein above in the section “Bacterial cell”, and may further comprise any of the modifications described in “Other modifications”. In particular, the first catechol 1,2-dioxygenase may be CatA-I and/or the second catechol 1,2-dioxygenase may be CatA-II as detailed above. In particular, the expression of the gene(s) encoding the benzoate 1,2-dioxygenase(s) may be under the control of specific first and/or second expression module(s) as described herein above in the section “Bacterial cell”. In particular, as described herein above, the first expression module may comprise the P_{14b} promoter or a variant thereof having at least 80% homology or identity thereto, and the strong translational coupler BCD2 or BCD10 or a variant thereof having at least 80% homology or identity thereto. In particular, as described herein above, the second expression module may comprise the P_{14b} promoter or a variant thereof having at least 80% homology or identity thereto and the strong translational coupler BCD10 or a variant thereof having at least 80% homology or identity thereto. In particular, as described herein above, the first expression module may comprise the Pm promoter variant ML1-17 or a variant thereof having at least 80% homology or identity thereto, the associated activator protein XylS or a functional variant thereof having at least 80% homology or identity thereto, and the strong translational coupler BCD2 or BCD10 or a variant thereof having at least 80% homology or identity thereto. In particular, as described herein above, the second expression module may comprise the Pm promoter or a variant thereof having at least 80% homology or identity thereto and the strong translational coupler BCD10 or a variant thereof having at least 80% homology or identity thereto.

In some embodiments, the first polynucleotide encoding a benzoate 1,2-dioxygenase, such as the benzoate 1,2-dioxygenase BenABC, comprises or consists of SEQ ID NO: 4, SEQ ID NO: 5, and SEQ ID NO: 6, or variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, said first polynucleotide further comprising a medium-strong constitutive promoter, such as the medium-strong constitutive promoter Ptac, comprising or consisting of SEQ ID NO: 14 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the second polynucleotide encoding a benzoate-1,2-dihydrodiol dehydrogenase, such as the benzoate-1,2-dihydrodiol dehydrogenase BenD, comprises or consists of SEQ ID NO: 8 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the third polynucleotide encoding a first catechol 1,2-dioxygenase, such as the catechol 1,2-dioxygenase CatA-I, comprises or consists of SEQ ID NO: 10 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, said third polynucleotide further comprising a first expression module comprising a first promoter, such as the medium-to-weak constitutive promoter P_{14b} comprising or consisting of SEQ ID NO: 49 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, and a first strong translational coupler, such as the translational coupler BCD10 comprising or consisting of SEQ ID NO: 16, or the translational coupler BCD2 comprising or consisting of SEQ ID NO: 22, or variants thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the third polynucleotide encoding a first catechol 1,2-dioxygenase, such as the catechol 1,2-dioxygenase CatA-I, comprises or consists of SEQ ID NO: 10 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, said third polynucleotide further comprising a first expression module comprising a first inducible promoter, such as the inducible promoter Pm variant ML1-17 comprising or consisting of SEQ ID NO: 18 or a variant thereof having at least 80% homology, such as at least 85%, such as at least 90%, such as at least 95% homology or identity thereto, an associated activator protein, such as the associated activator protein XylS, comprising or consisting of SEQ ID NO: 20 or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, and a first strong translational coupler, such as the strong translational coupler BCD10 comprising or consisting of SEQ ID NO: 16 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, or the strong translational coupler BCD2 comprising or consisting of SEQ ID NO: 22 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the fourth polynucleotide encoding a second catechol 1,2-dioxygenase, such as the catechol 1,2-dioxygenase CatA-II, comprises or consists of SEQ ID NO: 12 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, said fourth polynucleotide further comprising a second expression module comprising a second promoter, such as the medium-to-weak constitutive promoter P_{14b} comprising or consisting of SEQ ID NO: 49 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, and a second strong translational coupler, such as the strong translational coupler BCD10, comprising or consisting of SEQ ID NO: 16 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the fourth polynucleotide encoding a second catechol 1,2-dioxygenase, such as the catechol 1,2-dioxygenase CatA-II, comprises or consists of SEQ ID NO: 12 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, said fourth polynucleotide further comprising a second expression module comprising a second inducible promoter, such as the inducible Pm promoter, comprising or consisting of SEQ ID NO: 15 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, and a second strong translational coupler, such as the strong translational coupler BCD10, comprising or consisting of SEQ ID NO: 16 or a variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, the nucleic acid construct encodes the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenase CatA-I (SEQ ID NO: 9).

In some embodiments, the nucleic acid construct encodes the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenases CatA-I (SEQ ID NO: 9) and CatA-II (SEQ ID NO: 11).

In some embodiments, the nucleic acid construct encodes the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenase CatA-I (SEQ ID NO: 9), wherein BenABC is expressed under the control of the medium-strong constitutive promoter P_{tac} (SEQ ID NO: 15) and CatA-I is expressed under the control of the first expression module comprising or consisting of the medium-to-weak constitutive promoter P_{14b} (SEQ ID NO: 49), and the first strong translational coupler BCD2 (SEQ ID NO: 22) or BCD10 (SEQ ID NO: 16).

In some embodiments, the nucleic acid construct encodes the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenase CatA-I (SEQ ID NO: 9), wherein BenABC is expressed under the control of the medium-strong constitutive promoter P_{tac} (SEQ ID NO: 15) and CatA-I is expressed under the control of the first expression module comprising or consisting of the first inducible promoter Pm variant ML1-17 (SEQ ID NO: 18), the associated activator protein is XylS (SEQ ID NO: 21), and the first strong translational coupler BCD2 (SEQ ID NO: 22) or BCD10 (SEQ ID NO: 16).

In some embodiments, the nucleic acid construct encodes the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenases CatA-I (SEQ ID NO: 9) and CatA-II (SEQ ID NO: 11), wherein BenABC is expressed under the control of the medium-strong constitutive promoter P_{tac} (SEQ ID NO: 15) and CatA-I is expressed under the control of the first expression module comprising or consisting of the medium-to-weak constitutive promoter P_{14b} (SEQ ID NO: 49), and the first strong translational coupler BCD2 (SEQ ID NO: 22) or BCD10 (SEQ ID NO: 16), and wherein CatA-II is expressed under the control of the second expression module comprising or consisting of the medium-to-weak constitutive promoter P_{14b} (SEQ ID NO: 49) and the second strong translational coupler BCD10 (SEQ ID NO: 16).

In some embodiments, the nucleic acid construct encodes the benzoate 1,2-dioxygenase BenABC consisting of BenA (SEQ ID NO: 1), BenB (SEQ ID NO: 2) and BenC (SEQ ID NO: 2), the benzoate-1,2-dihydrodiol dehydrogenase BenD (SEQ ID NO: 7), and the catechol 1,2-dioxygenases CatA-I (SEQ ID NO: 9) and CatA-II (SEQ ID NO: 11), wherein BenABC is expressed under the control of the medium-strong constitutive promoter P_{tac} (SEQ ID NO: 15) and CatA-I is expressed under the control of the first expression module comprising or consisting of the first inducible promoter Pm variant ML1-17 (SEQ ID NO: 18), the associated activator protein XylS (SEQ ID NO: 21), and the first strong translational coupler BCD2 (SEQ ID NO: 22) or BCD10 (SEQ ID NO: 16), and wherein CatA-II is expressed under the control of the second expression module comprising or consisting of the inducible promoter Pm (SEQ ID NO: 15) and the second strong translational coupler BCD10 (SEQ ID NO: 16).

In some embodiments, one or more of the nucleic acids encoding each of the present activities, i.e. a benzoate 1,2-dioxygenase, a benzoate-1,2-dihydrodiol dehydrogenase, and catechol 1,2-dioxygenase, may be codon-optimized for said bacterial cell.

In some embodiments, each of the nucleic acids encoding each of the present activities, i.e. a benzoate 1,2-dioxygenase, a benzoate-1,2-dihydrodiol dehydrogenase, and catechol 1,2-dioxygenase, may be designed to be integrated within the genome of the cell or they may be within one or more vectors comprised within the cell.

In some embodiments, one or more of the nucleic acids encoding each of the present activities may be integrated in the genome of said cell. Methods for integrating a nucleic acid are well known in the art. Thus in some embodiments the activity of interest is encoded by introduction of a heterologous nucleic acid in the bacterial cell.

The heterologous nucleic acid encoding said activity may be codon-optimized, or may comprise features that can help improve the activity. Such modifications include, but are not limited to, the introduction of localization signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the activity of interest can be performed by methods known in the art. The skilled person will recognize that such methods include, but are not limited to: cloning and homologous recombination-based methods. Cloning methods may involve the design and construction of a plasmid e.g. in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the bacterial cell.

In some embodiments, the nucleic acids encoding the activities of interest may be present in high copy number.

In some embodiments, the nucleic acid construct further comprises or consists of one or more vectors, such as an integrative vector or a replicative vector. In some embodiments, the vector is a high copy replicative vector.

The bacterial cell may be as described herein.

### Kit of Parts

Also provided herein is a kit of parts comprising a bacterial cell, for example a bacterial cell as described herein, and/or a nucleic acid construct as described herein, and instructions for use.

In some embodiments, the kit comprises a bacterial cell that can be used in the methods for producing 2-fluoro-cis,cis-muconate described herein. In other embodiments, the kit comprises a nucleic acid construct that can be used to engineer a bacterial cell useful for the methods for producing 2-fluoro-cis,cis-muconate described herein. In some embodiments, the kit comprises a bacterial cell and a nucleic acid construct as described herein.

In some embodiments, the kit comprises a bacterial cell capable of producing 2-fluoro-cis,cis-muconate, wherein the bacterial cell expresses a benzoate 1,2-dioxygenase, a benzoate-1,2-dihydrodiol dehydrogenase and at least one catechol 1,2-dioxygenase. The yeast cell may be further modified as detailed in the section “Other modifications”.

In some embodiments, the kit comprises a bacterial cell capable of producing 2-fluoro-cis,cis-muconate, wherein the bacterial cell expresses a benzoate 1,2-dioxygenase, a benzoate-1,2-dihydrodiol dehydrogenase and at least two catechol 1,2-dioxygenases. The yeast cell may be further modified as detailed in the section “Other modifications”.

In some embodiments, the kit comprises a nucleic construct comprising a first polynucleotide encoding a benzoate 1,2-dioxygenase and further comprising a medium-strong constitutive promoter. In some embodiments, the kit comprises a nucleic construct comprising a second polynucleotide encoding a benzoate-1,2-dihydrodiol dehydrogenase. In some embodiments, the kit comprises a nucleic construct comprising a third polynucleotide encoding a first catechol 1,2-dioxygenase, a first expression module comprising a first promoter, said first expression module further comprising a first strong translational coupler. In some embodiments, the kit comprises a nucleic construct comprising a fourth polynucleotide encoding a second catechol 1,2-dioxygenase, a second expression module comprising a second promoter, said second expression module further comprising a second strong translational coupler. In some embodiments, the kit comprises a nucleic construct comprising the first, second and third polynucleotides described herein above. In some embodiments, the kit comprises a nucleic construct comprising the first, second, third and fourth polynucleotides described herein above.

In some embodiments, the kit comprises a nucleic construct comprising a first polynucleotide encoding a benzoate 1,2-dioxygenase and further comprising a medium-strong constitutive promoter. In some embodiments, the kit comprises a nucleic construct comprising a second polynucleotide encoding a benzoate-1,2-dihydrodiol dehydrogenase. In some embodiments, the kit comprises a nucleic construct comprising a third polynucleotide encoding a first catechol 1,2-dioxygenase, a first expression module comprising a first inducible promoter and a sequence encoding an associated activator protein, said first expression module further comprising a first strong translational coupler. In some embodiments, the kit comprises a nucleic construct comprising a fourth polynucleotide encoding a second catechol 1,2-dioxygenase, a second expression module comprising a second inducible promoter, said second expression module further comprising a second strong translational coupler. In some embodiments, the kit comprises a nucleic construct comprising the first, second and third polynucleotides described herein above. In some embodiments, the kit comprises a nucleic construct comprising the first, second, third and fourth polynucleotides described herein above.

In some embodiments, the kit comprises the nucleic acid construct as described herein and the bacterial cell to be modified. In some embodiments, the bacterial cell to be modified is a Pseudomonas putida cell, preferably a Pseudomonas putida KT2440 cell.

### EXAMPLES

### Materials and Methods

### Bacterial Strains and Culture Conditions

The bacterial strains employed in the present study are listed in Table 1, below. E. coli and P. putida were incubated at 37° C. and 30° C., respectively. For cell propagation and storage, routine cloning procedures, and during genome engineering manipulations, cells were grown in lysogeny broth (LB) medium (10 g/L tryptone, 5 g/L yeast extract, and 10 g/L NaCl). Liquid cultures were performed using either 50-ml centrifuge tubes with a medium volume of 5-10 mL, or in 500-mL Erlenmeyer flasks covered with cellulose plugs (Carl Roth, Karlsruhe, Germany) and with a medium volume of 50 mL. All liquid cultures were agitated at 250 rpm (MaxQ™8000 incubator; ThermoFisher Scientific, Waltham, MA, USA). Solid culture media contained an additional 15 g/L agar. Selection of plasmid-harboring cells was achieved by adding kanamycin (Km), gentamicin (Gm), or streptomycin (Sm) when required at 50 μg mL⁻¹, 10 μg mL⁻¹, and 50 μg mL⁻¹, respectively. For phenotypic characterizations in microtiter plates as well as fermentations in shaken flasks, P. putida was pre-grown in LB medium, and the experiments were performed in de Bont synthetic minimal medium (Hartmans, Smits et al. 1989) additionally buffered with 10 g/L 3-(N-morpholino)propanesulfonic acid (MOPS) at pH 7.0 and supplemented with different carbon compounds as explained in the text.

For shaken flask experiments, LB precultures were harvested by centrifugation at 4000×g for 10 min, washed with de Bont medium without the addition of any carbon compound, and resuspended in the final media of the experiment at the desired start-OD_{600 nm}. Cell growth was monitored by measuring the absorbance at 630 nm (for plate reader experiments) or 600 nm (for shaken flask experiments). The optical density at 630 nm (OD_{630 nm}) was estimated from plate reader absorbance values by multiplying the values by 1.751, a value which had been previously determined with pure water using the method described by Lampinen, Raitio et al. (2012). Alternatively, cell growth was monitored by measuring the absorbance at 630 nm (for plate reader experiments with ELx808, BioTek Instruments; Winooski, VT, USA) or 600 nm (for shaken flask experiments) and estimating the optical density at 600 nm (OD_{600 nm}) from plate reader absorbance values by multiplying the values by correlation factors, which had been previously determined for the employed microtiter plate-readers and spectrophotometers. Biomass concentrations (cell dry weight, g_{CDW} L⁻¹) in shaken flask experiments were derived from OD_{600 nm} measurements with a correlation factor 0.35, previously determined for the spectrophotometer employed with exponentially growing P. putida KT2440.

Comparative phenotypical characterizations and quantifications of green fluorescence for bioreporters were performed in 96-well plates in a Synergy HI plate reader (BioTek Instruments; Winooski, VT, USA). Therefore, the LB preculture was diluted 1:100 in the respective screening medium (de Bont medium supplemented with various organic compounds). Fluorescence was measured at an excitation wavelength of 488 nm and an emission wavelength of 588 nm. The transcription and translation initiation strengths of various expression systems tested in bioreporter experiments are provided by the relative normalized fluorescence of GFP at an OD_{630 nm} value of 1.0 for each culture, which represents a point within the mid-exponential growth phase of P. putida. For this purpose, the absolute GFP emission values were normalized by the OD_{630 nm} (˜1.0) and divided by the normalized fluorescence value of the P_{tac} reporter strain.

TABLE 1
Bacterial strains used in this study.

| Strain [Alias] | Relevant characteristics | Reference or source |
|---|---|---|
| Escherichia coli | | |
| DH5α λ pir | Cloning host; F⁻ λ⁻ endA1 | Platt, Drescher et al. |
| | glnX44(AS) thiE1 recA1 relA1 | (2000) |
| | spoT1 gyrA96(Nal^{R}) rfbC1 deoR | |
| | nupG Φ80(lacZΔM15) Δ(argF- | |
| | lac)U169 hsdR17(r_{K}⁻ m_{K}⁺), λ pir | |
| | lysogen | |
| Pseudomonas | | |
| P. putida KT2440 | Wild-type strain, derived from P. | Bagdasarian, Lurz et |
| | putida mt-2 (Worsey and Williams | al. (1981) |
| | 1975) cured of the TOL plasmid | |
| | pWW0 | |
| P. knackmussii | Wild-type strain | Stolz, Busse et al. |
| (Pseudomonas sp. | | (2007) |
| B13) | | |
| P. putida P_{tac}→benABC *¹ | P. putida KT2440 with the | This study |
| | chromosomal benABC gene cluster | P_{tac}: de Boer, |
| | put under the control of the | Comstock et al. |
| | constitutive P_{tac} promoter. The | (1983) |
| | native, validated 5′ UTR of the | benA 5′ UTR |
| | benA gene were kept unchanged, | definition: Perez- |
| | providing a predicted translation | Pantoja, Kim et al. |
| | rate of 220. | (2015) |
| P. putida P_{tac}→benD *¹ | P. putida KT2440 with the | This study |
| | chromosomal benD gene under the | P_{tac}: de Boer, |
| | control of the constitutive P_{tac} | Comstock et al. |
| | promoter. The native 28 bp | (1983) |
| | upstream of the benD gene were | |
| | kept unchanged, providing a | |
| | putative translation initiation | |
| | sequence with a predicted | |
| | translation rate of 818. | |
| P. putida P_{EM7}→catA *¹ | P. putida KT2440 with the the | This study |
| | constitutive P_{EM7} promoter | |
| | chromosomally inserted upstream | |
| | of the catA gene. The native 29 bp | |
| | upstream of the catA gene were | |
| | kept unchanged, providing a | |
| | putative translation initiation | |
| | sequence with a predicted | |
| | translation rate of 3211. | |
| P. putida ΔcatBC *¹ | P. putida KT2440 with both | This study |
| | chromosomal genes catB and catC | |
| | deleted | |
| P. putida ΔcatBC::catA *¹ | P. putida KT2440 with the genes | This study |
| | catB and catC replaced with an | |
| | additional copy of catA. The | |
| | predicted translation rate of catA | |
| | with the catB 5′ UTR is 1934. | |
| P. putida ΔcatBC::catA- | P. putida KT2440 with the genes | This study |
| II *¹ | catB and catC replaced with an | |
| | additional copy of catA-II. The | |
| | predicted translation rate of catA-II | |
| | with the catB 5′ UTR is 503. | |
| P. putida ΔcatBC | | This study |
| P_{tac}→benABC *¹ | | P_{tac}: de Boer, |
| | | Comstock et al. |
| | | (1983) |
| P. putida ΔcatBC | | This study |
| P_{tac}→benD *¹ | | P_{tac}: de Boer, |
| | | Comstock et al. |
| | | (1983) |
| P. putida ΔcatBC::catA | | This study |
| P_{tac}→benD *¹ | | P_{tac}: de Boer, |
| | | Comstock et al. |
| | | (1983) |
| P. putida ΔcatBC::catA-II | | This study |
| P_{tac}→benD *¹ | | P_{tac}: de Boer, |
| | | Comstock et al. |
| | | (1983) |
| P. putida | P. putida KT2440 with the | This study |
| Pm-BCD10→catA *² | chromosomal gene catA put under | BCD10: Mutalik, |
| | the control of the inducible Pₘ | Guimaraes et al. |
| | promoter and the translational | (2013) |
| | coupling sequence BCD10 with | |
| | predicted translation strengths of | |
| | 28177 (SD1) and 331 (SD2). | |
| P. putida | P. putida KT2440 with the | This study |
| Pm-BCD10→catA-II *² | chromosomal gene catA put under | BCD10: Mutalik, |
| | the control of the inducible Pₘ | Guimaraes et al. |
| | promoter and the translational | (2013) |
| | coupling sequence BCD10 with | |
| | predicted translation strengths of | |
| | 28177 (SD1) and 134 (SD2). | |
| Pm-BCD10→catA | | This study |
| Pm-BCD10→catA-II *² | | |
| P. putida [Pmuc3] *² | | This study |
| Pm-BCD10→catA | | |
| Pm-BCD10→catA-II | | |
| P_{tac}→benABC | | |
| P. putida [Pmuc3x] *² | Strain Pmuc3 with the xylS gene | This study |
| xylS/Pm(ML1-17)- | with its native regulatory | BCD10: Mutalik, |
| BCD10→catA | sequences chromosomally | Guimaraes et al. |
| Pm-BCD10→catA-II | integrated upstream of and | (2013) |
| P_{tac}→benABC | encoded on the opposite DNA | Pm(ML1-17): Bakke, |
| | strand of catA, which is controlled | Berg et al. (2009) |
| | by the Pm variant ML1-17. | |
| Pm-BCD10→catA | pBen1 is a suicide plasmid | This study |
| Pm-BCD10→catA-II | harboring HAs for the | The use of the |
| pBen1 pSEVA438 *² | chromosomal integration of | landing site was |
| | additional copies of Pm- | described previously |
| | BCD10→benABC and Pm- | in Wirth, Kozaeva et |
| | BCD10→benDK-(catA-II), into a | al. (2019) |
| | landing site between PP_0013 and | |
| | PP_5421 | |
| P. putida [Pmuc4] *³ | Pmuc3 with the catabolite | This study |
| Pm-BCD10→catA | repression control gene crc | |
| Pm-BCD10→catA-II | deleted. | |
| P_{tac}→benABC Δcrc | | |
| [Pmuc3F] *³ | Pmuc3 with the porin-like protein | This study |
| Pm-BCD10→catA | encoding gene nicP-I put under the | Pm(ML1-17): Bakke, |
| Pm-BCD10→catA-II | control of the inducible Pₘ promoter | Berg et al. (2009) |
| Pm-BCD10→nicP-I | variant ML1-17 and the | |
| P_{tac}→benABC | translational coupling sequence | |
| | BCD10 with predicted translation | |
| | strengths of 31045 (SD1) and 72 | |
| | (SD2). | |
| [Pmuc3E2] *³ | Pmuc3 with the benzoate transport | This study |
| Pm-BCD10→catA | protein encoding gene benE-II put | Pm(ML1-17): Bakke, |
| Pm-BCD10→catA-II | under the control of the inducible | Berg et al. (2009) |
| Pm-BCD10→benE-II | Pₘ promoter variant ML1-17 and | |
| P_{tac}→benABC | the translational coupling sequence | |
| | BCD10 with predicted translation | |
| | strengths of 31045 (SD1) and 1594 | |
| | (SD2). | |
| [Pmuc3K] *³ | Pmuc3 with the benzoate MFS | This study |
| Pm-BCD10→catA | transporter encoding gene benk | Pm(ML1-17): Bakke, |
| Pm-BCD10→catA-II | put under the control of the | Berg et al. (2009) |
| Pm-BCD10→benK | inducible Pₘ promoter variant ML1- | |
| P_{tac}→benABC | 17 and the translational coupling | |
| | sequence BCD10 with predicted | |
| | translation strengths of 31045 | |
| | (SD1) and 541 (SD2). | |
| Pm-BCD2→catA | Chromosomal benABC gene | This study |
| P_{tac}→benABC *³ | cluster put under the control of the | BCD2: Mutalik, |
| | constitutive P_{tac} promoter. | Guimaraes et al. |
| | Chromosomal gene catA put under | (2013) |
| | the control of the inducible Pₘ | |
| | promoter and the translational | |
| | coupling sequence BCD2 with | |
| | predicted translation strengths of | |
| | 28177 (SD1) and 18908 (SD2). | |
| Pm-BCD2→catA-II | Chromosomal benABC gene | This study |
| P_{tac}→benABC *³ | cluster put under the control of the | BCD2: Mutalik, |
| | constitutive P_{tac} promoter. | Guimaraes et al. |
| | Chromosomal gene catA-II put | (2013) |
| | under the control of the inducible | |
| | Pₘ promoter and the medium- | |
| | strong translational coupling | |
| | sequence BCD2 with predicted | |
| | translation strengths of 28177 | |
| | (SD1) and 7687 (SD2). | |
| P_{tac}-BCD10→benABC | Pmuc3 with the native 5′-UTR of | This study |
| Pm-BCD10→catA | benABC replaced with BCD10. | |
| Pm-BCD10→catA-II *³ | | |
| P. putida [Pmuc4vhb] *³ | Pmuc4 with the Vitreoscilla | This study |
| Pm-BCD10→catA | stercoraria vhb gene inserted into | |
| Pm-BCD10→catA-II | the crc locus under control of Pm | |
| P_{tac}→benABC | and BCD10 | |
| Δcrc::vhb | | |
| P. putida [Pmuc4tat- | Pmuc4 with the V. stercoraria vhb | This study |
| vhb] *³ | gene inserted into the crc locus | uxpA signal peptide: |
| Pm-BCD10→catA | under control of Pm and BCD10, | Putker, Tommassen- |
| Pm-BCD10→catA-II | and fused at the 5′ end to a type II | van Boxtel et al. |
| P_{tac}→benABC | secretion system signal sequence | (2013) |
| Δcrc::tat-vhb | derived from the P. putida KT2440 | |
| | gene uxpA | |
| P. putida [Pmuc4c] *³ | Pmuc4 with the xylS gene | This study |
| P_{14c}-BCD10→xylS | chromosomally integrated under | |
| Pm-BCD10→catA | P_{14c}-BCD10 control upstream of | |
| Pm-BCD10→catA-II | and encoded on the opposite DNA | |
| P_{tac}→benABC Δcrc | strand of catA | |
| P. putida [Pmuc4g] *³ | Pmuc4 with the xylS gene | This study |
| P_{14g}-BCD10→xylS | chromosomally integrated under | |
| Pm-BCD10→catA | P_{14g}-BCD10 control upstream of | |
| Pm-BCD10→catA-II | and encoded on the opposite DNA | |
| P_{tac}→benABC Δcrc | strand of catA | |
| P. putida [Pmuc5] *⁴ | Pmuc4 with benABC under the | This study |
| Pm-BCD10→catA | control of the constitutive P_{tac} | |
| Pm-BCD2→catA-II | promoter and the native 5′-UTR of | |
| P_{tac} -BCD10→benABC | benABC replaced with BCD10. | |
| Δcrc | | |
| P. putida [Pmuc6] *⁴ | Pmuc5 with catA and catA-II put | This study |
| J23115-BCD10→catA | under the control of the medium- | J12115: iGEM Part |
| J23115-BCD10→catA-II | weak, consitutive promoter J23115 | BBa_J23115 |
| P_{tac}-BCD10→benABC | and the translational coupling | |
| Δcrc | sequence BCD10. | |
| P. putida [Pmuc7] *⁴ | Pmuc5 with catA and catA-II put | This study |
| J23108-BCD10→catA | under the control of the medium- | J12108: iGEM Part |
| J23108-BCD10→catA-II | weak, consitutive promoter J23108 | BBa_J23108 |
| P_{tac}-BCD10→benABC | and the translational coupling | |
| Δcrc | sequence BCD10. | |
| P. putida [Pmuc8] *⁴ | Pmuc5 with catA and catA-II put | This study |
| J23114-BCD10→catA | under the control of the medium- | J12115: iGEM Part |
| J23114-BCD10→catA-II | weak, consitutive promoter J23114 | BBa_J23114 |
| P_{tac}-BCD10→benABC | and the translational coupling | |
| Δcrc | sequence BCD10. | |
| P. putida [Pmuc9] *⁴ | Pmuc5 with catA and catA-II put | This study |
| P_{14b}-BCD10→catA | under the control of the medium- | P_{14b}: Zobel, |
| P_{14b}-BCD10→catA-II | weak, consitutive promoter P_{14b} | Benedetti et al. |
| P_{tac}-BCD10→benABC | and the translational coupling | (2015) |
| Δcrc | sequence BCD10. | |
| P. putida [Pmuc10] *⁵ | Pmuc9 with intact sequence of the | This study |
| P_{14b}-BCD10→catA | gene crc, and with benABC under | |
| P_{14b}-BCD10→catA-II | the transcriptional control of the | |
| P_{tac} →benABC | constitutive P_{tac} promoter and the | |
| | native 5′-UTR controlling | |
| | translation initiation. | |
| P. putida [Pmuc11] *⁵ | Pmuc9 with benABC under the | This study |
| P_{14b}-BCD10→catA | transcriptional control of the | |
| P_{14b}-BCD10→catA-II | constitutive P_{tac} promoter and the | |
| P_{tac}→benABC | native 5′-UTR controlling | |
| Δcrc | translation initiation. | |
| P. putida [Pmuc12] *⁵ | Pmuc9 with intact sequence of the | This study |
| P_{14b}-BCD10→catA | gene crc. | |
| P_{14b}-BCD10→catA-II | | |
| P_{tac}-BCD10→benABC | | |
| P. putida [Pmuc13] *⁵ | Pmuc3 with catA-II put under the | |
| Pm-BCD10→catA | control of the medium-weak, | |
| P14b-BCD10→catA-Il | consitutive promoter P_{14b} and the | |
| P_{tac}→benABC | translational coupling sequence | |
| | BCD10. | |
| P. putida [Pmuc14] *⁵ | Pmuc3 with xylS integrated into the | This study |
| Pm-BCD10→catA | chromosome upstream of catA-II | |
| xylS/Pm-BCD10→catA-II | under the control of the | |
| P_{tac}→benABC | strong, consitutive promoter P_{14g}, | |
| | and catA-II under the translational | |
| | control of BCD10 in addition to the | |
| | transcriptional control by Pm. | |

*¹ Strain created in the first round of strain design
*² Strain created in the second round of strain design
*³ Strain created in the third round of strain design
*⁴ Strain created in the fourth round of strain design
*⁵ Strain created in the fifth round of strain design

### Cloning Procedures and Plasmid Construction

All plasmids and oligonucleotides used in this work are listed in Table 2, below. Uracil-excision (USER) cloning (Cavaleiro, Kim et al. 2015) was used for the construction of all plasmids. The AMUSER tool was employed for the design of oligonucleotides (Genee, Bonde et al. 2015). All genetic manipulations followed the protocol published previously (Wirth, Kozaeva et al. 2019). DNA fragments employed in assembly reactions were amplified using the Phusion™ U high-fidelity DNA polymerase (Thermo Fisher Scientific, Waltham, Massachusetts, USA) according to the manufacturer's specifications. The identity and correctness of all plasmids and DNA constructs were confirmed by sequencing. For genotyping experiments after cloning procedures and genome manipulations, colony PCRs were performed using the commercial OneTaq™ master mix (New England BioLabs; Ipswich, MA, USA) according to the manufacturer's instructions. E. coli DH5α λ pir was employed for all cloning purposes. Chemically competent E. coli cells were prepared and transformed with plasmids according to well-established protocols (Elder 1983). P. putida was rendered electro-competent following the protocol of Choi, Kumar et al. (2006), i.e., by washing the biomass from saturated (16 h) LB medium cultures with 0.3 M sucrose, followed by transformation with plasmids by electroporation with a voltage of 2.5 kV, 25 μF capacitance and 200) resistance in a Gene Pulser Xcell™ Electroporation System (Bio-Rad Laboratories, Hercules, CA, USA).

TABLE 2
Plasmids used in this study.

| | | Reference or |
|---|---|---|
| Plasmid | Relevant characteristics^{a} | source |
| pGNW2 | Suicide vector used for genetic manipulations in | Wirth, |
| | Gram-negative bacteria; oriT, traJ, lacZα, | Kozaeva et al. |
| | oriV(R6K), P14g-BCD2→msfGFP; Km^{R} | (2019) |
| PSNW2 | Derivative of vector pGNW2 with the translation | Volke, Friis et |
| | initiation sequence of msfGFP replaced by the | al. (2020) |
| | very strong translational coupling sequence BCD2 | |
| pSEVA628S | Helper plasmid; oriV(RK2), xylS, | Martínez- |
| | Pm→I-SceI; GmR | Garćía, |
| | | Aparicio et al. |
| | | (2015) |
| pSEVA228 | Plasmid used to supply XylS for chromosomal Pm | (Martínez- |
| | promoters; oriV(RK2), xylS, | Garćía, |
| | Pm; Km^{R} | Aparicio et al. |
| | | 2015) |
| pSEVA228.2 | Plasmid used to supply XylS^{Thr45} (XylS.2) for | XylS.2: Ramos |
| | chromosomal Pm promoters; oriV(RK2), xylS.2, | et al. (1990) |
| | Pm; Km^{R} | |
| PQURE6•H | Conditionally-replicating vector; derivative of | Volke, Friis et |
| | vector pJBSD1 carrying | al. (2020) |
| | XylS/Pm→I-SceI and P_{14g}-BCD2→mRFP; Gm^{R} | |
| pGNW2•P_{tac} | Derivative of pGNW2 carrying homology arms | This study |
| →benABC | (HAs) to replace the native P_{ben} promoter | P_{tac}: de Boer, |
| | upstream of benA with the P_{tac} promoter | Comstock et |
| | sequence P. putida KT2440 | al. (1983) |
| pGNW2•P_{tac} | Derivative of pGNW2 carrying HAs to insert the | This study |
| →benD | P_{tac} promoter sequence upstream of benD P. | P_{tac}: de Boer, |
| | putida KT2440 | Comstock et |
| | | al. (1983) |
| pGNW2•P_{EM7} | Derivative of pGNW2 carrying HAs to insert the | This study |
| →catA | P_{EM7} promoter sequence upstream of catA P. | |
| | putida KT2440 | |
| pGNW2•Pm- | Derivative of pGNW2 carrying HAs to insert the | This study |
| BCD10•catA | Pm promoter and the translational coupling | BCD10: |
| | sequence BCD10 upstream of catA P. putida | Mutalik, |
| | KT2440 | Guimaraes et |
| | | al. (2013) |
| pGNW2•Pm- | Derivative of pGNW2 carrying HAs to insert the | This study |
| BCD2→catA | Pm promoter and the translational coupling | BCD2: Mutalik, |
| | sequence BCD2 upstream of catA P. putida | Guimaraes et |
| | KT2440 | al. (2013) |
| pGNW2•xylS/ | Derivative of pGNW2 carrying HAs to insert the | This study |
| Pm(ML1-17)- | xylS gene with its native regulatory sequences, | ML1-17: |
| BCD10→catA | the Pm promoter variant ML1-17, and the | Bakke, Berg et |
| | translational coupling sequence BCD10 upstream | al. (2009) |
| | of catA P. putida KT2440 | BCD10: |
| | | Mutalik, |
| | | Guimaraes et |
| | | al. (2013) |
| pGNW2•P_{14g}- | Derivative of pGNW2 carrying HAs to insert the | This study |
| BCD10→xylS/ | xylS gene under the control of P_{14g} and BCD10, | ML1-17: |
| Pm- | the Pm promoter, and the translational coupling | Bakke, Berg et |
| BCD10→catA | sequence BCD10 upstream of catA P. putida | al. (2009) |
| | KT2440 | |
| pGNW2•P_{14c}- | Derivative of pGNW2 carrying HAs to insert the | This study |
| BCD10→xylS/ | xylS gene under the control of P_{14c} and BCD10, | BCD10: |
| Pm- | the Pm promoter, and the translational coupling | Mutalik, |
| BCD10→catA | sequence BCD10 upstream of catA P. putida | Guimaraes et |
| | KT2440 | al. (2013) |
| pGNW2•Pm- | Derivative of pGNW2 carrying HAs to insert the | This study |
| BCD10→catA- | Pm promoter and the translational coupling | BCD10: |
| II | sequence BCD10 upstream of catA-II P. putida | Mutalik, |
| | KT2440 | Guimaraes et |
| | | al. (2013) |
| pGNW2•Pm- | Derivative of pGNW2 carrying HAs to insert the | This study |
| BCD2→catA- | Pm promoter and the translational coupling | BCD2: Mutalik, |
| II | sequence BCD2 upstream of catA-II P. putida | Guimaraes et |
| | KT2440 | al. (2013) |
| PSNW2•Pm- | Derivative of pGNW2 carrying HAs to insert the | This study |
| BCD10→nicP- | Pm promoter and the translational coupling | BCD10: |
| I | sequence BCD10 upstream of nicP-I P. putida | Mutalik, |
| | KT2440 | Guimaraes et |
| | | al. (2013) |
| PSNW2•Pm- | Derivative of pGNW2 carrying HAs to insert the | This study |
| BCD10→benE- | Pm promoter and the translational coupling | BCD10: |
| II | sequence BCD10 upstream of benE-II P. putida | Mutalik, |
| | KT2440 | Guimaraes et |
| | | al. (2013) |
| PSNW2•Pm- | Derivative of pGNW2 carrying HAs to insert the | This study |
| BCD10→benK | Pm promoter and the translational coupling | BCD10: |
| | sequence BCD10 upstream of benK P. putida | Mutalik, |
| | KT2440 | Guimaraes et |
| | | al. (2013) |
| pGNW2•Δcrc | Derivative of pGNW2 carrying HAs to delete crc in | This study |
| | P. putida KT2440 | |
| pGNW2•Δcrc::vhb | Derivative of pGNW2 carrying HAs to delete crc in | This study |
| | P. putida KT2440 and replace it with the | |
| | sequence of the Vitreoscilla stercoraria vhb gene | |
| | under control of Pm and BCD10 | |
| pGNW2•Δcrc::tat-vhb | Derivative of pGNW2 carrying HAs to delete crc in | This study |
| | P. putida KT2440 and replace it with the | uxpA signal |
| | sequence of the V. stercoraria vhb gene under | peptide: |
| | control of Pm and BCD10, and fused at the 5′ end | Putker, |
| | to a type II secretion system signal sequence | Tommassen- |
| | derived from the P. putida KT2440 gene uxpA | van Boxtel et |
| | | al. (2013) |
| pGNW•ΔcatBC | Derivative of pGNW2 carrying HAs to delete | This study |
| | catBC in P. putida KT2440 | |
| pGNW•ΔcatBC::catA | Derivative of pGNW2 carrying HAs to delete | This study |
| | catBC in P. putida KT2440 replace it with an | |
| | additional copy of catA | |
| pGNW•ΔcatBC::catA-II | Derivative of pGNW2 carrying HAs to delete | This study |
| | catBC in P. putida KT2440 replace it with an | |
| | additional copy of catA-II | |
| pGNW2•P_{tac}- | Derivative of pGNW2 carrying homology arms | This study |
| BCD10→benABC | (HAs) to replace the native P_{ben} promoter | P_{tac}: de Boer, |
| | upstream of benA with the P_{tac} promoter | Comstock et |
| | sequence, and the native 5′-UTR with the | al. (1983) |
| | translational coupling sequence BCD10 in P. | BCD10: |
| | putida KT2440. | Mutalik, |
| | | Guimaraes et |
| | | al. (2013) |
| pGNW2•J23108- | Derivative of pGNW2 carrying homology arms | This study |
| BCD10→catA | (HAs) to insert the constitutive J23108 promoter | J12108: iGEM |
| | and the translational coupling sequence BCD10 | Part |
| | upstream of catA in P. putida KT2440. | BBa_J23108 |
| | | BCD10: |
| | | Mutalik, |
| | | Guimaraes et |
| | | al. (2013) |
| pGNW2•J23108- | Derivative of pGNW2 carrying homology arms | This study |
| BCD10→catA-II | (HAs) to insert the constitutive J23108 promoter | J12108: iGEM |
| | and the translational coupling sequence BCD10 | Part |
| | upstream of catA-II in P. putida KT2440. | BBa_J23108 |
| | | BCD10: |
| | | Mutalik, |
| | | Guimaraes et |
| | | al. (2013) |
| pGNW2•J23114- | Derivative of pGNW2 carrying homology arms | This study |
| BCD10→catA | (HAs) to insert the constitutive J23114 promoter | J12114: iGEM |
| | and the translational coupling sequence BCD10 | Part |
| | upstream of catA in P. putida KT2440. | BBa_J23114 |
| | | BCD10: |
| | | Mutalik, |
| | | Guimaraes et |
| | | al. (2013) |
| pGNW2•J23114- | Derivative of pGNW2 carrying homology arms | This study |
| BCD10→catA-II | (HAs) to insert the constitutive J23114 promoter | J12114: iGEM |
| | and the translational coupling sequence BCD10 | Part |
| | upstream of catA-II in P. putida KT2440. | BBa_J23115 |
| | | BCD10: |
| | | Mutalik, |
| | | Guimaraes et |
| | | al. (2013) |
| pGNW2•J23115- | Derivative of pGNW2 carrying homology arms | This study |
| BCD10→catA | (HAs) to insert the constitutive J23115 promoter | J12115: iGEM |
| | and the translational coupling sequence BCD10 | Part |
| | upstream of catA in P. putida KT2440. | BBa_J23114 |
| | | BCD10: |
| | | Mutalik, |
| | | Guimaraes et |
| | | al. (2013) |
| pGNW2•J23115- | Derivative of pGNW2 carrying homology arms | This study |
| BCD10→catA-II | (HAs) to insert the constitutive J23115 promoter | J12115: iGEM |
| | and the translational coupling sequence BCD10 | Part |
| | upstream of catA-II in P. putida KT2440. | BBa_J23114 |
| | | BCD10: |
| | | Mutalik, |
| | | Guimaraes et |
| | | al. (2013) |
| pGNW2•P_{14b}- | Derivative of pGNW2 carrying homology arms | This study |
| BCD10→catA | (HAs) to insert the constitutive P_{14b} promoter and | P_{14b}: Zobel, |
| | the translational coupling sequence BCD10 | Benedetti et al. |
| | upstream of catA in P. putida KT2440. | (2015) |
| | | BCD10: |
| | | Mutalik, |
| | | Guimaraes et |
| | | al. (2013) |
| pGNW2•P_{14b}- | Derivative of pGNW2 carrying homology arms | P_{14b}: Zobel, |
| BCD10→catA-II | (HAs) to insert the constitutive P_{14b} promoter and | Benedetti et al. |
| | the translational coupling sequence BCD10 | (2015) |
| | upstream of catA-II in P. putida KT2440. | BCD10: |
| | | Mutalik, |
| | | Guimaraes et |
| | | al. (2013) |
| pGNW2•P_{14g}→ | Derivative of pGNW2 carrying homology arms | This study |
| xylS/Pm- | (HAs) to insert the Pm promoter and the | BCD10: |
| BCD2→catA-II | translational coupling sequence BCD10, as well | Mutalik, |
| | as the gene sequence of xylS under the control of | Guimaraes et |
| | the strong, constitutive promoter P_{14g}, upstream of | al. (2013) |
| | catA-II in P. putida KT2440. | |
| pS628-BCD1 | oriV(RK2), xylS, Pm-BCD1→msfGFP; Gm^{R} | This study |
| →GFP | | BCD1: Mutalik, |
| | | Guimaraes et |
| | | al. (2013) |
| pS628-BCD2 | oriV(RK2), xylS, Pm-BCD2→msfGFP; Gm^{R} | This study |
| →GFP | | BCD2: Mutalik, |
| | | Guimaraes et |
| | | al. (2013) |
| pS628-BCD7 | oriV(RK2), xylS, Pm-BCD7→msfGFP, Gm^{R} | This study |
| →GFP | | BCD7: Mutalik, |
| | | Guimaraes et |
| | | al. (2013) |
| pS628-BCD10 | oriV(RK2), xylS, Pm-BCD10→msfGFP; Gm^{R} | This study |
| →GFP | | BCD10: |
| | | Mutalik, |
| | | Guimaraes et |
| | | al. (2013) |
| PS628(ML1- | oriV(RK2), xylS, Pm(ML1-17)-BCD10→msfGFP; | This study |
| 17)-BCD10 | Gm^{R} | ML1-17: |
| →GFP | | Bakke, Berg et |
| | | al. (2009) |
| | | BCD10: |
| | | Mutalik, |
| | | Guimaraes et |
| | | al. (2013) |
| pS62P_{tac}- | oriV(RK2), P_{tac}-BCD10→msfGFP; Gm^{R} | This study |
| BCD10 | | P_{tac}: de Boer, |
| →GFP | | Comstock et |
| | | al. (1983) |
| | | BCD10: |
| | | Mutalik, |
| | | Guimaraes et |
| | | al. (2013) |
| PS62P_{EM7}- | oriV(RK2), P_{EM7}-BCD10→msfGFP; Gm^{R} | This study |
| BCD10 | | BCD10: |
| →GFP | | Mutalik, |
| | | Guimaraes et |
| | | al. (2013) |
| pS62P_{14g}- | oriV(RK2), P_{14g}-BCD10→msfGFP; Gm^{R} | This study |
| BCD10 | | P_{14g}: Zobel, |
| →GFP | | Benedetti et al. |
| | | (2015) |
| | | BCD10: |
| | | Mutalik, |
| | | Guimaraes et |
| | | al. (2013) |
| PS62P_{14d}- | oriV(RK2), P_{14d}-BCD10→msfGFP; Gm^{R} | This study |
| BCD10 | | P_{14d}: Zobel, |
| →GFP | | Benedetti et al. |
| | | (2015) |
| | | BCD10: |
| | | Mutalik, |
| | | Guimaraes et |
| | | al. (2013) |
| pS62P_{cat}- | oriV(RK2), catR, P_{cat}-BCD10→msfGFP, Gm^{R} | This study |
| BCD10 | | BCD10: |
| →GFP | | Mutalik, |
| | | Guimaraes et |
| | | al. (2013) |
| pS62P_{ben}-BCD10 | oriV(RK2), benR, P_{ben}-BCD10→msfGFP; Gm^{R} | This study |
| →GFP | | |
| pSEVA634::PKB_1379 | oriV(pBBR1), laclq, P_{trc}→PKB_1379, Gm^{R} | This study |
| pSEVA634::PKB_2107 | oriV(pBBR1), laclq, P_{trc}→PKB_2107, Gm^{R} | This study |
| pSEVA634::PKB_3273 | oriV(pBBR1), laclq, P_{trc}→PKB_3273, Gm^{R} | This study |
| pSEVA438 | oriV(pBBR1), xylS, Pm, Sm^{R} | Martínez- |
| | | Garćía, |
| | | Aparicio et al. |
| | | (2015) |

^{a}Antibiotic markers: Km, kanamycin; Nal, nalidixic acid; Gm, gentamicin; Km,
kanamycin; Sm, streptomycin

### Metabolite Analysis

For the detection of extracellular metabolites, supernatants were obtained via centrifugation of bacterial culture broths for 2 min at 13,000×g. 2-Fluoromuconic acid (2-FMA), cis,cis-muconic acid (ccMA), catechol, 3-fluorocatechol (3-FC), 4-fluorocatechol (4-FC), 2-fluorobenzoic acid (2-FBz), and 3-fluorobenzoic acid (3-FBz) were quantified using a Dionex 3000 HPLC system equipped with a Zorbax Eclipse Plus C18 column (Agilent Technologies, Santa Clara, CA, USA) that was heated to 30° C., and a guard column from Phenomenex. Separation was achieved with a mobile phase consisting of 0.05% (w/v) acetic acid and varying amounts of acetonitrile. The total runtime per sample was 8.3 min (with a separation time of 8.0 min), during which the fraction of acetonitrile was increased from 1% to 3% within the first 3 min, followed by a steady increase to 20% within 12 s, and a further steady increase to 75% within 4 min. From 7.2 to 7.5 min, the acetonitrile concentration was held at 75% and subsequently reduced to 1% within 18 s. The column was then equilibrated again at 1% acetonitrile for further 30 s before injecting the next sample. The flow rate was set to 1 ml min⁻¹, and the injection volume was 0.75 μl. Elution of the compounds was detected by UV light at wavelengths of 210 nm, 240 nm, 280 nm, and 300 nm. HPLC data were processed using the Chromeleon 7.1.3 software (Thermo Fisher Scientific), and compound concentrations were calculated using calibration curves.

### Software

Data manipulations and calculations were performed in Microsoft Excel 2016 and OriginPro 2019 (OriginLab Corporation). Figures and Illustrations were created in OriginPro 2019 and Adobe Illustrator 2020. Geneious Prime 2020.1.2 (Biomatters Ltd.) served as a database for any kind DNA sequences, to design plasmids and constructs, and to analyze Sanger sequencing results. Maximum exponential growth rates μₘₐₓ in Example 1 were calculated using the tool GrowthRates published by Hall, Acar et al. (2014). Allternatively, maximum exponential growth rates μₘₐₓ were determined by Gaussian process regression using the Python-based tool deODorizer published by Swain et al. (2016). The prediction of translation initiation strengths for 5′ untranslated region (5′ UTR) mRNA sequences was performed using the online RBS Calculator v2.1 which can be found at https://www.denovodna.com/software/predict_rbs_calculator. The results are given in arbitrary units (au) on the RBS Calculator v2.0 scale, representing the relative strength of translation.

### Data Analysis

Biomass-specific 3-FBz consumption rates qₛ and 2-FMA production rates qₚ were determined over the timeframes of fermentations in which there was 3-FBz detectable in the culture media with the following equations:

$${q}_{S} = \frac{1}{\overline{X}} \frac{Δ S}{Δ t} \qquad ( 1 )$$

$${q}_{P} = \frac{1}{\overline{X}} \frac{Δ P}{Δ t} . \qquad ( 2 )$$

qₛ: biomass-specific substrate consumption rate (g_{3FBz} g_{CDW}⁻¹ h⁻¹)
X̅: the average biomass concentration between two sampling timepoints (g_{CDW} L⁻¹)
ΔS: Difference in substrate concentration between two sampling timepoints (g_{3FBz})
Δt: time between two sampling points
qₚ: biomass-specific product formation rate (g_{2FMA} g_{CDW}⁻¹ h⁻¹)
ΔP: Difference in product concentration between two sampling timepoints (g_{2FMA})

The qₛ and qₚ rates displayed in Table 5 are averages of the differential values determined for the individual time intervals.

### Example 1—Wild-Type P. putida KT2440 Converts Fluoro-Substituted Benzoic Acids into Fluoro-Muconic Acid

To examine the suitability of the ortho-cleavage pathway enzymes in P. putida for the bioconversion of 2- or 3-FBz to 2-FMA, both P. knackmussii and P. putida KT2440 were cultured in microtiter plates with minimal medium supplemented with 30 mM glucose (or 30 mM potassium benzoate) and varying concentrations of metabolites involved in the bioconversion process (Figures A-C). Maximum exponential growth rates (μₘₐₓ) observed, determined in the early exponential phase for each growth curve individually, are listed in Table 3, below.

TABLE 3
Growth rates of selected strains used in this study. Cells
were precultured in LB medium supplemented. The experiment
was performed in 96-well microtiter plates with each well
containing 200 μl de Bont medium with 10 g/L MOPS and
varying concentrations of carbon sources and metabolites
involved in the bioconversion process. Growth rates were
determined by performing an exponential regression to the
growth curves within the early exponential phase. Every
regression had a coefficient of determination (R²) of >0.998.

| | Maximum growth rate μₘₐₓ |
|---|---|
| Strain/media additives | (h⁻¹) |
| P. putida KT2440 | |
| 30 mM glucose | 0.60 |
| 30 mM K-benzoate | 0.37 |
| 30 mM glucose, 0.5 mM catechol | 0.72 |
| 30 mM glucose, 1.0 mM catechol | 0.77 |
| 30 mM glucose, 2.0 mM catechol | 0.78 |
| 30 mM glucose, 0.5 mM 3-F-catechol | 0.72 |
| 30 mM glucose, 1.0 mM 3-F-catechol | 0.65 |
| 30 mM glucose, 2.0 mM 3-F-catechol | 0.39 |
| 30 mM glucose, 0.5 mM 4-F-catechol | 0.77 |
| 30 mM glucose, 1.0 mM 4-F-catechol | 0.64 |
| 30 mM glucose, 2.0 mM 4-F-catechol | 0.48 |
| 30 mM glucose, 10 mM K-2-F-benzoate | 0.57 |
| 30 mM glucose, 15 mM K-2-F-benzoate | 0.53 |
| 30 mM glucose, 20 mM K-2-F-benzoate | 0.48 |
| 30 mM glucose, 5.0 mM K-3-F-benzoate | 0.36 |
| 30 mM glucose, 10 mM K-3-F-benzoate | 0.13 |
| 30 mM glucose, 15 mM K-3-F-benzoate | no growth |
| 30 mM glucose, 20 mM K-3-F-benzoate | no growth |
| P. knackmussii | |
| 30 mM glucose | 0.38 |
| 30 mM K-benzoate | no growth |
| 30 mM glucose, 0.5 mM catechol | 0.36 |
| 30 mM glucose, 1.0 mM catechol | 0.33 |
| 30 mM glucose, 2.0 mM catechol | 0.31 |
| 30 mM glucose, 0.5 mM 3-F-catechol | 0.28 |
| 30 mM glucose, 1.0 mM 3-F-catechol | 0.17 |
| 30 mM glucose, 2.0 mM 3-F-catechol | 0.11 |
| 30 mM glucose, 0.5 mM 4-F-catechol | 0.34 |
| 30 mM glucose, 1.0 mM 4-F-catechol | 0.32 |
| 30 mM glucose, 2.0 mM 4-F-catechol | 0.25 |
| 30 mM glucose, 10 mM K-2-F-benzoate | 0.25 |
| 30 mM glucose, 15 mM K-2-F-benzoate | 0.22 |
| 30 mM glucose, 20 mM K-2-F-benzoate | no growth |
| 30 mM glucose, 5.0 mM K-3-F-benzoate | 0.01 |
| 30 mM glucose, 10 mM K-3-F-benzoate | no growth |
| 30 mM glucose, 15 mM K-3-F-benzoate | no growth |
| 30 mM glucose, 20 mM K-3-F-benzoate | no growth |
| Pmuc3 pSEVA228 | |
| 30 mM glucose | 0.64 |
| 30 mM K-benzoate, 0.5 mM 3-mBz | 0.16 |
| 30 mM glucose, 0.5 mM catechol | 0.77 |
| 30 mM glucose, 1.0 mM catechol | 0.78 |
| 30 mM glucose, 2.0 mM catechol | 0.86 |
| 30 mM glucose, 0.5 mM 3-F-catechol | 0.61 |
| 30 mM glucose, 1.0 mM 3-F-catechol | 0.48 |
| 30 mM glucose, 2.0 mM 3-F-catechol | 0.22 |
| 30 mM glucose, 0.5 mM 4-F-catechol | 0.55 |
| 30 mM glucose, 1.0 mM 4-F-catechol | 0.54 |
| 30 mM glucose, 2.0 mM 4-F-catechol | 0.35 |
| 30 mM glucose, 5.0 mM K-3-F-benzoate | 0.36 |
| 30 mM glucose, 10 mM K-3-F-benzoate | 0.33 |
| 30 mM glucose, 15 mM K-3-F-benzoate | 0.32 |
| 30 mM glucose, 20 mM K-3-F-benzoate | 0.30 |

Within the course of 50 h, no growth was detectable for P. knackmussii on benzoate as the sole source of carbon, while P. putida grew with a μₘₐₓ of 0.37 h⁻¹. With glucose as the primary source of carbon and energy, P. putida grew with a maximum rate μₘₐₓ of 0.6 h⁻¹, which is about 1.6 times higher than that of P. knackmussii (μₘₐₓ, 0.38 h⁻¹). In the presence of 2-FBz, both species demonstrated some degree of sensitivity to the compound, which became more prominent with increasing concentrations. However, while P. putida still grew with 80% of its maximum rate on glucose and without any noticeable effect on the duration of the lag phase in the presence of 20 mM 2-FBz, P. knackmussii was unable to grow at all within the course of the experiment (50 h) and showed significantly increased lag phases at 10 mM and 15 mM of 2-FBz. At 15 mM 2-FBz, P. knackmussii grew with 58% of the μₘₐₓ observed on only glucose. The detrimental impact of the second bioconversion substrate 3-FBz on the growth of both species was more severe. P. putida was able to grow in the presence of maximum 10 mM 3-FBz with a substantially reduced μₘₐₓ value. Even at 5 mM, the growth of P. knackmussii was almost completely impaired and no growth was observable at concentrations of 10 mM.

Depending on the position of the fluoro-substitution on the benzoate derivative, the three 1,2-dihydroxybenzenes pyrocatechol (here referred to as catechol), 3-FC, and 4-FC are produced as intermediary metabolites. No toxic effect was observed in the presence of up to 2 mM catechol on P. putida. In fact, with increasing catechol concentrations, μₘₐₓ increased by up to 30%. For P. knackmussii, catechol demonstrated a slightly toxic effect, evident in a decrease in μₘₐₓ with increasing catechol concentrations. The physiological response of the two species to the two fluorinated 1,2-dihydroxybenzene derivatives, 3-FC and 4-FC, was more pronounced, but was reflected in different growth characteristics for each strain. While 3-FC affected predominantly the cells' growth rate, the presence of 4-FC had a much more prominent effect on the duration of lag phases. However, in both cases, P. putida demonstrated a higher resistance to the toxic effects of both 3-FC and 4-FC than P. knackmussii.

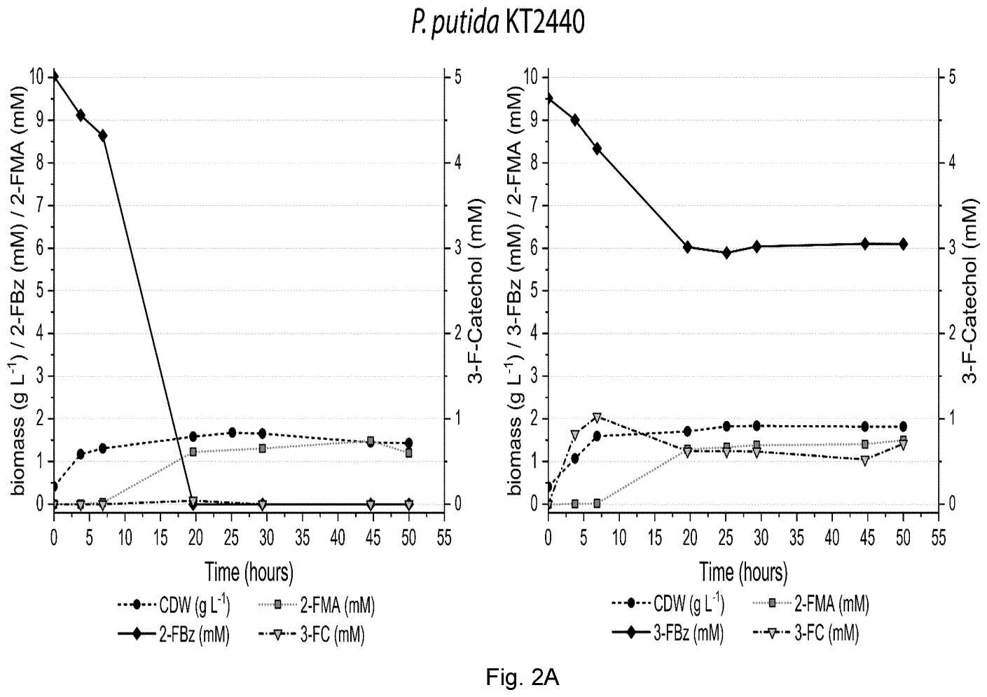

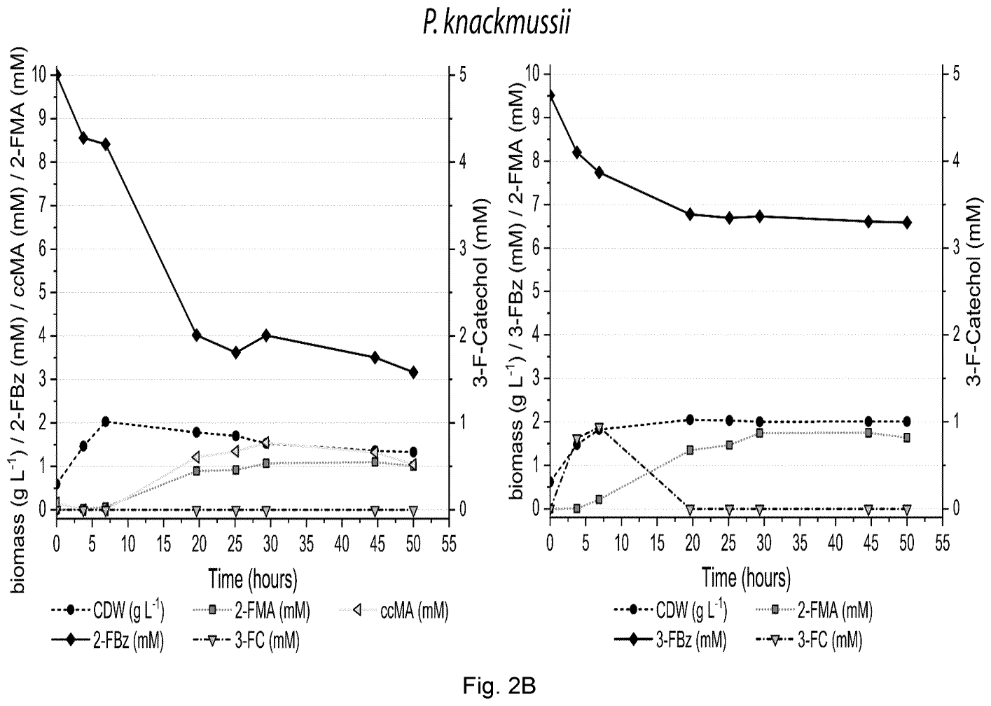

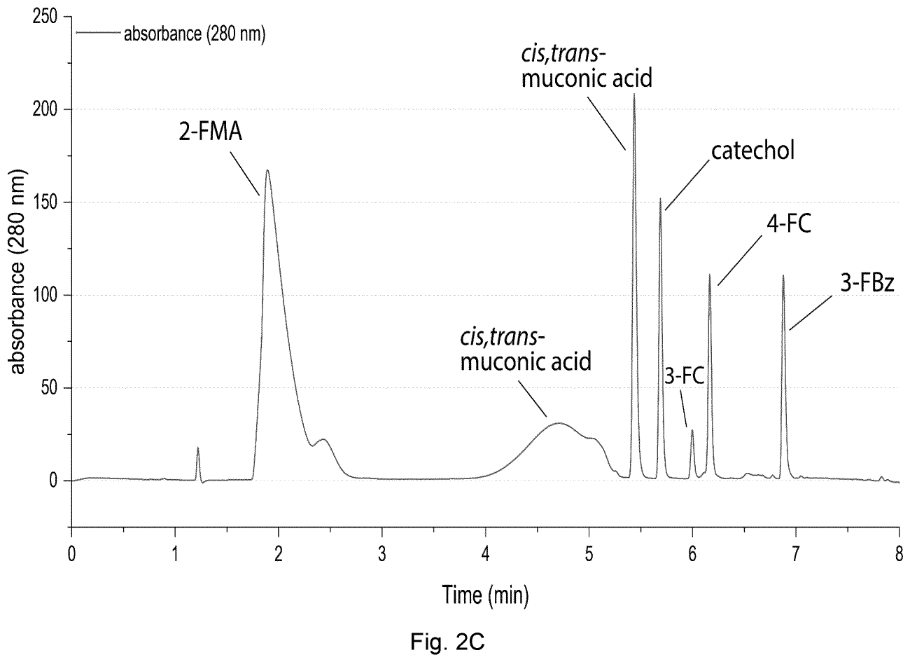

After establishing the physiological response of both P. knackmussii and P. putida to the chemical compounds involved in the bioconversion of FBz to 2-FMA, the two species were subjected to a fermentation experiment in shaken flasks containing synthetic de Bont medium with 10 g/L MOPS at pH 7.0, and supplemented with 30 mM glucose as well as 10 mM of 2-FBz or 3-FBz (FIGS. 2A-C). P. putida completely consumed 2-FBz within 20 h with a substrate uptake rate qₛ of 0.47 mmol g_{CDW}⁻¹ h⁻¹. 2-FMA was the only compound detectable after substrate depletion, at a concentration of 1.34 mM and produced at a maximum product formation rate qₚ of 0.06 mmol g_{CDW}⁻¹ h⁻¹. Neither 3-FC nor 4-FC were detected at any point in time. After an initial consumption of 2-FBz with a qₛ of 0.26 mmol g_{CDW}⁻¹ h⁻¹ for about 20 h, P. knackmussii stopped taking up any further substrate (final concentration of 4 mM). Out of the 6 mM substrate consumed, about 1 mM 2-FMA was produced at a product formation rate qₚ of 0.05 mmol g_{CDW}⁻¹ h⁻¹. Furthermore, ccMA could be detected in the culture supernatant at a maximum concentration of 1.55 mM after 30 h, which continued to be consumed slowly but not completely until the end of the fermentation (50 h). For both bacterial species, the amount of 2-FMA produced represents 14% of the 3-FBz consumed (product yield coefficient Y_{SP}=0.14 mol mol⁻¹).

Exposed to 3-FBz, P. putida consumed the substrate up until a time between 7 and 20 h with a qₛ of 0.162 mmol g_{CDW}⁻¹ after which the 3-FBz concentration in the culture supernatant remained constant. Within the first 7 h, all 3-FBz consumed was converted into 3-FC until it reached a concentration of 1 mM. In the course of the next 13 h, 40% of 3-FC in the supernatant was converted into 2-FMA, combined with a fraction of additionally consumed 3-FBz at a combined product formation rate qₚ of 0.06 mmol g_{CDW}⁻¹ h⁻¹. At the end of the fermentation, 3.48 mM 3-FBz had been converted into 1.38 mM 2-FMA, corresponding to a product yield Y_{SP} of 0.41 mol mol-1, as well as 0.52 mM 3-FC. The combined concentrations of metabolites constituting the 1,2-dioxygenation route (2-FMA and 3-FC) add up to 1.90 mM, representing 55% of the consumed 3-FBz. Considering its previously established toxicity, the accumulation of 3-FC likely represents a major bottleneck for an efficient and prolonged bioconversion process in P. putida. Also with P. knackmussii, 3-FBz consumption occurred only within the first 7-20 h of the fermentation, with a qₛ of 0.090 mmol g_{CDW}⁻¹ h⁻¹. After an initial increase of 3-FC up to 0.94 mM in the culture supernatant, the catechol derivative was completely re-consumed until 20 h. From a total concentration of 2.93 mM 3-FBz consumed, P. knackmussii produced 1.64 mM 2-FMA [corresponding to 56% of the substrate (Y_{SP}: 0.41 mol mol-1)] with a rate qₚ of 0.05 mM g_{CDW}⁻¹ h⁻¹. In none of the conditions tested significant amounts of 4-FC could be detected in the culture supernatants. The enzyme machinery performing the di-oxygenation on fluorinated catechol derivatives that both species are equipped with thus seems to demonstrate a higher efficiency towards 4-FC compared to 3-FC.

With the asymmetrical use of the non-productive 1,2-dioxygenation route when using 2-FBz as substrate for the bioconversion, almost 90% of fluorine gets lost, in contrast to about 50% with 3-FBz as substrate. The 3-F-substituted benzoate consequently represents the most suitable precursor compound to produce 2-FMA. A faster growth rate, higher biomass yield on glucose, higher qₛ and qₚ values, and a higher resistance to toxic effects of pathway intermediates make P. putida stand out as the bacterial platform better suited to perform this bioconversion.

### Example 2—Strain Engineering of P. putida KT2440 to Increase Bioconversion Performance with 3-FBz

As apparent from metabolite analyses of the supernatant during bioconversion experiments with P. putida and 3-FBz, the last biochemical step of the upper ortho-cleavage pathway, conversion of 3-FC into 2-FMA, represents a major bottleneck for a balanced conversion without the accumulation of toxic 3-FC. Following an approach commonly employed in the field of microbial strain engineering, a first set of genome manipulations was performed on P. putida KT2440 with the aim of increasing the enzyme availability of different steps of the bioconversion pathway, especially that of the two catechol 1,2-dioxygenases, by means of constitutive promoters (the strains are listed in Table 1, above).

These modifications comprised the replacement of the native P_{ben} promoter upstream of benA with the constitutive P_{tac} promoter sequence [predicted translation strength with the native 5′ untranslated region (UTR): 220], the insertion of P_{tac} upstream of the 28 bp 5′ UTR (predicted translation strength: 818) of benD, and the insertion of the P_{EM7} promoter sequence upstream of the 29 bp 5′ UTR of catA (predicted translation strength: 3211).

Approaches to insert a regulatory element comprising P_{tac} and a translational coupling sequence [bi-cistronic designs—BCD2 or BCD10, published by Mutalik, Guimaraes et al. (2013)] upstream of catA [translation strengths predicted using the RBS Calculator v2.1; BCD2: 28177 (SD1) and 18908 (SD2); BCD10: 28177 (SD1) and 331 (SD2)] or catA-II [predicted translation strengths BCD2: 28177 (SD1) and 7687 (SD2); BCD10: 28177 (SD1) and 134 (SD2)] failed due to the consistent emergence of mutations within the −35 region of P_{tac} in the cloning host E. coli harboring the respective genome editing plasmid, suggesting a toxic effect of catechol 1,2-dioxygenase overexpression under some conditions.

As an alternative strategy to increase catechol 1,2-dioxygenase activity, the three catA homologues from P. knackmussii were cloned into the replicable plasmid pSEVA634 under the control of the IPTG-inducible LacIq/P_{trc} system and inserted into P. putida KT2440. Following the strategy employed by Kohlstedt, Starck et al. (2018), further P. putida KT2400 strains were engineered with in-frame deletions of the two genes catB and catC to avoid (3-F-)cis,cis-muconate degradation, as well as variants where the catBC gene sequences were replaced by additional copies of catA or catA-II, putting them under the control of the native CatR/P_{cat} regulatory system (with predicted translation rates of 1934 for catA, and 503 for catA-II). These modifications were also combined with the aforementioned P_{tac}→benD design, which had been found to enhance productivities within the two upstream reactions of the ortho-cleavage pathway in preliminary experiments (not shown).

All ‘first generation’ strains (Table 1, above) were subjected to a bioconversion experiment in 500 ml Erlenmeyer flasks filled with 50 ml de Bont medium supplemented with 10 g/L MOPS (pH 7.0), 30 mM glucose and 10 mM 3-FBz. Within 24 h, all strain cultures reached a terminal state in which no further 3-FBz was consumed and all extracellular metabolites measured, with the exception of minor concentrations of 4-FC that continued to be consumed, remained at constant concentrations in the culture supernatants. These concentrations of the three most relevant metabolites of the bioconversion pathway 3-FBz, 3-FC, and 2-FMA are compared between each member of the first generation strains, as well as wild-type P. putida and P. knackmussii, in FIG. 3. A comparison of these final states of the fermentations allows to draw conclusions about the balance of biochemical activities involved in the conversion of 3-FBz into 2-FMA. As mentioned previously, the pathway in wild-type P. putida is not optimized to process halogenated benzoate derivatives, which is apparent as an accumulation of fluorinated catechol intermediates. This accumulation is decreased by exchanging the native P_{ben} promoter driving the expression of the ben gene cluster with a P_{tac} promoter. The insertion of P_{tac} upstream of benD increases 3-FC formation to an about five-fold higher concentration compared to P. putida wildtype, indicating a rate-limiting role of BenD towards the formation of 3-FC. However, such an activity increase within the upper two reactions of the pathway without increased catechol detoxification is detrimental for cell viability and bioconversion efficiency.

This apparent bottleneck was addressed by means of a catA overexpression by the constitutive P_{EM7} promoter with putative high transcription initiation strength. The constitutive overexpression of catA, however, had detrimental effects on the catechol 1,2-dioxygenase activity, leading to an almost complete accumulation of all 3-FBz entering the 1,2-dioxygenation route in the form of 3-FC. The deletion of catBC also lead to a higher accumulation of 3-FC in all strains that had been subject to this genomic manipulation. Additional expression of benABC or benD by P_{tac} brought about comparable effects in a ΔcatBC background as with catBC intact. Also the implementation of additional copies of catA or catA-II under P_{cat} control in place of catBC did not show any positive effect on the conversion of 3-FC into 2-FMA.

In addition to >2 mM 3-FC and <0.93 mM 2-FMA, each of the seven ΔcatBC strains accumulated a new by-product eluting at 4.0 min (HPLC) with an absorption maximum at 277.5 nm and with a molecular weight of 299.06 g mol⁻¹ (as determined by LC-MS). The molecular weight and fragmentation pattern corresponded to either of the two C₁₂H₁₃NO₃ compounds 3-[Bis(carboxymethyl)amino]methyl-2,4-dihydroxybenzoic acid or 3-[Bis(carboxymethyl)amino]methyl-4,5-dihydroxybenzoic acid (data not shown). The greatest detriment on the bioconversion performance was caused by the expression of the three P. knackmussii catA orthologues from replicable plasmids. While 3-FBz uptake was greatly enhanced with most of the substrate having been consumed within 24 h, almost 50% of the initial 3-FBz was converted into 3-FC, with final concentrations of about 5 mM.

Of the manipulations tested, only P_{tac}→benABC had a balancing effect on the enzyme activities within the pathway, and efforts to increase the enzyme abundance of catechol 1,2-dioxygenase appeared to further reduce the rate at which its reaction is performed.

### Example 3—Characterization of Suitable Expression Systems for a Balanced Conversion of 3-FBz to 2-FMA

To get a better understanding of the effects that were caused by the defined genomic manipulation within first generation strains, all regulatory sequences involved in the strain engineering process were subjected to a comparative analysis. To this end, a set of standardized reporter plasmids was constructed utilizing pSEVA62X as backbone (Martinez-Garcia, Aparicio et al. 2014). Besides the low-copy number origin of vegative replication oriV(RK2) and a gentamicin resistance determinant, each plasmid harbors the msfGFP reporter gene under the control of i) the bi-cistronic translational coupling sequence BCD10 (Mutalik, Guimaraes et al. 2013) preceded by varying sequences of promoters relevant in this study, or ii) the XylS/Pm expression system followed by varying bi-cistronic translational coupling sequences to compare their relative translation initiation strength in P. putida.

A complete list of reporter constructs tested is summarized in Table 2, above. P. putida KT2440 harboring each of the reporter plasmid was cultured in de Bont medium supplemented with 30 mM glucose and, if applicable, varying concentrations of inducer compounds. A relative comparison of the effects of different bi-cistronic translational coupling sequences (BCD) is shown in FIG. 4.

The five chosen BCD variants showed the same relative effect on translation initiation as published in E. coli by Mutalik, Guimaraes et al. (2013), with BCD1 and BCD2 providing the highest GFP expression, followed by BCD7, BCD10, and BCD20. With the set of translational coupling sequences tested, expression of target genes can be tuned within an about three-fold range. With the BCD sequences behaving comparable as originally published for E. coli, it is expected that any of the other published variants can be chosen to provide translation initiation at even lower rates.

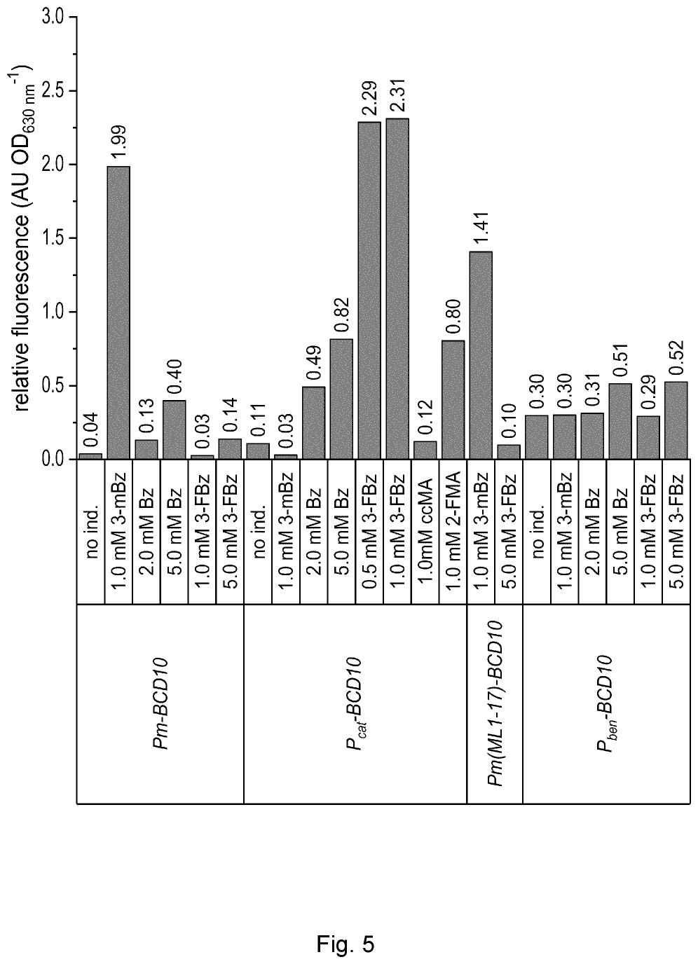

FIG. 5 shows a comparison of the transcription strength provided by the native CatR/P_{cat} promoter system, the BenR/P_{ben} system, as well as the XylS/Pm system and its variant XylS/Pm(ML1-17) which was reported to have a higher expression strength than Pm (Bakke, Berg et al. 2009). Compared are the induction strengths in the presence of the native XylS/Pm inducer 3-methyl benzoate (3-mBz), the native substrate of the ortho-cleavage pathway benzoate (Bz), the bioconversion substrate 3-FBz, and, for CatR/Pcₐt, its co-inducer compounds ccMA and 2-FMA.

With both 3-mBz and 3-FBz as inducers, GFP expression under Pm(ML1-17) control was only 70% of the expression provided by Pm. The XylS/Pm system was induced to 94% by 1 mM of 3-mBz as compared to 5 mM of the inducer (data not shown). With Bz and 3-FBz on the other hand, there was an almost linear dependency between inducer concentration and GFP expression within the range between 1.0 and 5.0 mM. Even with 5.0 mM inducer concentration, the GFP expression from Pm amounted to only 20% (for Bz) and 7% (for 3-FBz) of the one brought about by 1.0 mM 3-mBz. With 1.0 mM of 3-FBz, the expression strength was close to the basal, uninduced expression. The CatR/P_{cat} system was most strongly and fully induced already in the presence of 0.5 mM 3-FBz. The supplementation of 1.0 mM 3-mBz caused no response at all and with 5 mM Bz, only 35% of GFP fluorescence was observed as compared to the induction by 3-FBz. The system furthermore responded 6.7-fold stronger to 1.0 mM 2-FMA as compared to 1.0 mM ccMA. The fact that CatR/P_{cat} responds much more strongly to 3-FBz supplied in the medium than to 2-FMA suggests a more efficient uptake of 3-FBz which gets converted intracellularly to 2-FMA. The stronger response to 3-FBz and 2-FMA compared to Bz and ccMA could be caused by either a stronger interaction of CatR with the fluoro-substituted muconic acid or the stronger intracellular accumulation of 2-FMA, since ccMA is further consumed by the CatR/Pcₐt-regulated CatB enzyme.

The BenR/P_{ben} system displayed a high basal expression without the addition of any inducer. No further increase in GFP expression could be observed with 1.0 mM 3-mBz, 2.0 mM Bz, and 1.0 mM 3-FBz. Only with 5 mM of Bz or 3-FBz, induction increased by 70% compared to the uninduced system. It should be noted that the expression of BenR in wild-type P. putida KT2440 is negatively controlled by the translational repressor Crc which might have caused a decreased response of the BenR/P_{b}en system in the presence of glucose as the main source of carbon and energy.

Next, the inducible promoter systems BenR/P_{ben}, CatR/P_{cat}, and XylS (and its single substitution variants XylS_{Thr45} and XylS_{Val288})/Pm were compared under bioconversion conditions (with 5 mM 3-FBz) to the constitutive promoters P_{tac}, P_{EM7}, and P_{14g} (FIG. 6). The two XylS variants XylS_{Thr45} and XylS_{Val233} had been previously described as responding more strongly to 3-substituted Bz derivatives. The observed transcription initiation rates ranked as follows (values represent the quotient of the respective OD_{630 nm}-normalized fluorescence with the one of P_{tac}): P_{14g} (2.51)>CatR/P_{cat} (2.09)>P_{tac} (1.0)>P_{EM7} (0.57)>BenR/P_{ben} (0.52)>XylS_{Thr45}/Pm (0.29)>XylS/Pm (0.14)>XylS_{VAl233}/Pm (0.11)>XylS/Pm(ML1-17) (0.10).

The expression strengths of the promoter systems employed in the first round of strain engineering on P. putida KT2440 in the presence of 5 mM 3-FBz provide explanations for the phenotypic responses observed in the engineered strains. The expression of benABC by means of P_{tac} represents an about 2-fold overexpression compared to the native control by BenR/P_{ben}. A more complicated picture is drawn by the manipulations of catA(-II) expression by various expression modules. Any effort to increase the expression of catA or catA-II resulted in the increased accumulation of catechol 1,2-dioxygenase substrate 3-FC, suggesting a decrease in the respective biochemical activity.

This effect was particularly pronounced with the plasmid-based expression of P. knackmussii catA orthologues. With a reported copy number in P. putida of 30±7, the expression levels are expected to exceed those of any of the expression systems employed for the chromosomal manipulations. In the cloning host E. coli, the catA transcript levels provided by the chosen constitutive promoters are expected to be even higher or equal to those in P. putida under full induction of CatR/P_{cat} exposed to 3-FBz, as the plasmids are maintained at copy numbers of ˜10-15. In addition to that, CatA levels are further increased by the effect of the translational coupling sequences which are predicted to increase the translation initiation rate by a factor of 15 (BCD2) or 10 (BCD10) compared to the native catA translation initiation sequence (see Table 1, above).

In E. coli, the very high expression levels of catA appear to be toxic, thus providing a selection pressure to mutate the promoter sequences, while in P. putida, high CatA(-II) enzyme levels caused a disruption of catechol 1,2-dioxygenase activity. The given data on expression system performances suggests that a reduction in catA expression might lead to an increased bioconversion efficiency in P. putida.

### Example 4—Implementation of a Dynamic Catechol 1,2-Dioxygenase Expression Control Enables a Balanced, Complete Conversion of 3-FBz into 2-FMA

Guided by the observations made on the effects of genetic manipulations within first generation strains and the expression system comparison, a set of new strains was designed, in which catA and/or catA-II was put under the control of the XylS/Pm system that responds weakly to the bioconversion substrate 3-FBz and showed no induction in the absence of any inducer compound. The latter was thought to facilitate the cloning process in E. coli. Because 3-FBz-induced XylS/Pm provides transcript levels at a rate of only 7% compared to CatR/P_{cat} (see FIG. 6), the native translation initiation sequence of catA and catA-II were replaced by BCD10, which increases translation initiation by about 10-fold. The changes within the regulatory sequences for catA and catA-II were furthermore combined with P_{tac}→benABC, the integration of xylS into the chromosome under control of its native regulatory sequences, or the integration of the suicide plasmid pBen1, which harbors additional copies of the benABC and benDK-(catA-II) operons each under control of Pm-BCD10, into a landing site between PP_0013 and PP_5421. Strains without xylS integrated into the chromosome were additionally transformed with pSEVA228 or pSEVA438 (complementing pBen1) to provide the activator XylS expressed from a plasmid.

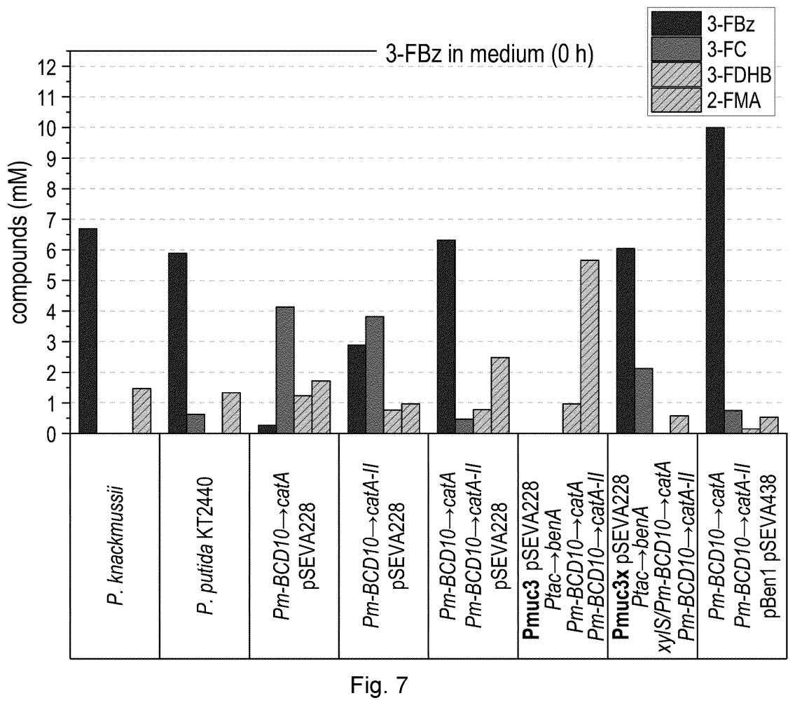

All strains created within the second round of strain engineering are listed in Table 1, above. Shaken flask bioconversion fermentations were performed in minimal medium with 30 mM glucose and 10 mM 3-FBz and the extracellular metabolites were analyzed via HPLC after 24 h of cultivation (see FIG. 7). While the individual replacement of regulatory sequences for catA and catA-II with Pm-BCD10 led to an increased formation of 3-FC, both manipulations combined resulted in reduced 3-FC concentrations in the culture supernatant, as well as an increase in 2-FMA concentration by about two-fold compared to wild-type P. putida KT2440. At the sampling time point, about 50% of 3-FBz were still left in the medium. The 3-FBz uptake rate was further enhanced by the implementation of P_{tac}→benABC (the resulting strain was named Pmuc3), which lead to a complete consumption of the substrate and a 2-FMA yield of about 50% without the accumulation of 3-FC. The remarkable performance of Pmuc3 was only observed when providing XylS from a plasmid (here pSEVA228) as indicated by the performance of strain Pmuc3x which has xylS integrated into the chromosome. This suggests that higher levels of XylS are necessary to saturate the expression of Pm-BCD10→catA and Pm-BCD10→catA-II which are located at distant positions within the P. putida chromosome. The integration of pBen1 in a strain that has both catA paralogues controlled by Pm-BCD10 significantly disrupted the bioconversion performance, apparent in a reduced 3-FBz uptake as well as an accumulation of 3-FC that is comparable to wild-type P. putida KT2440.

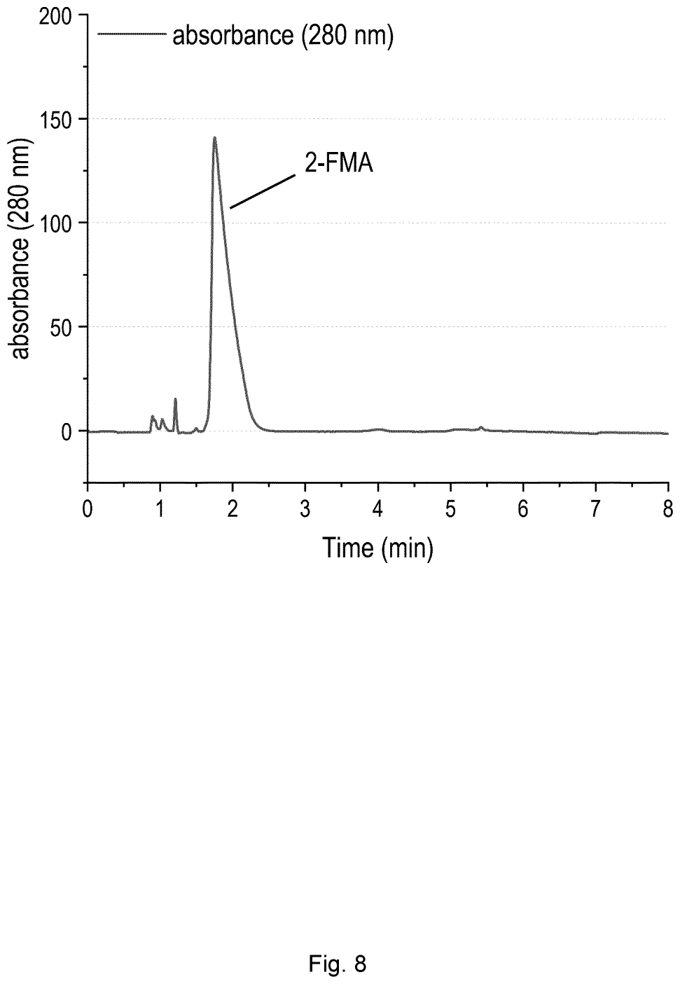

With 2-FMA as the only metabolite detected in the supernatant of Pmuc3 pSEVA228, the culture broth was subjected to the purification protocol published for ccMA by Vardon, Rorrer et al. (2016). This enabled the purification of about 30 mg 2-FMA from 40 ml of culture broth, corresponding to about 94% of 2-FMA initially measured in the culture supernatant. The chromatogram of an HPLC analysis with a UV-absorbance at 280 nm of the purified product is shown in FIG. 8.

### Example 5—Further Strain Engineering Involving Genetic “Off-Pathway” Targets Further Enhances Bioconversion Productivity while Maintaining the Biochemical Balance

With Pmuc3 harboring pSEVA228, a suitable set of genetic manipulation had been established that enables a well-balanced conversion of 3-FBz into 2-FMA without the accumulation of by-products. Using Pmuc3 as a genetic background, a further set of engineered strains was designed with the aim of further increasing the overall productivity of the bioconversion process.

As a first target the gene encoding the global catabolite repression control regulator Crc was deleted, which had been shown to exhibit post-transcriptional control over the BenR activator as well as to bind to certain mRNA targets of genes involved in the degradation of aromatic compounds. The rationale behind a crc deletion in Pmuc3 (resulting in strain Pmuc4) was to further increase the expression of benD, whose transcriptional control is unclear, as well as to enhance 3-FBz uptake since Crc was shown to have a repressing effect on the expression of a diverse range of porins and transporters. Because of the repressing effect that Crc reportedly has on benABC, its deletion was also combined with Pm-BCD10→catA and Pm-BCD10→catA-II without changing the regulation of benABC.

The three genes located within the ben gene cluster encoding membrane proteins that are thought to be involved in benzoate uptake, benK (transporter), benE-II (transporter), and nicP-I (also named benF, a porin), were also chosen as targets for expression control by Pm(ML1-17)-BCD10.

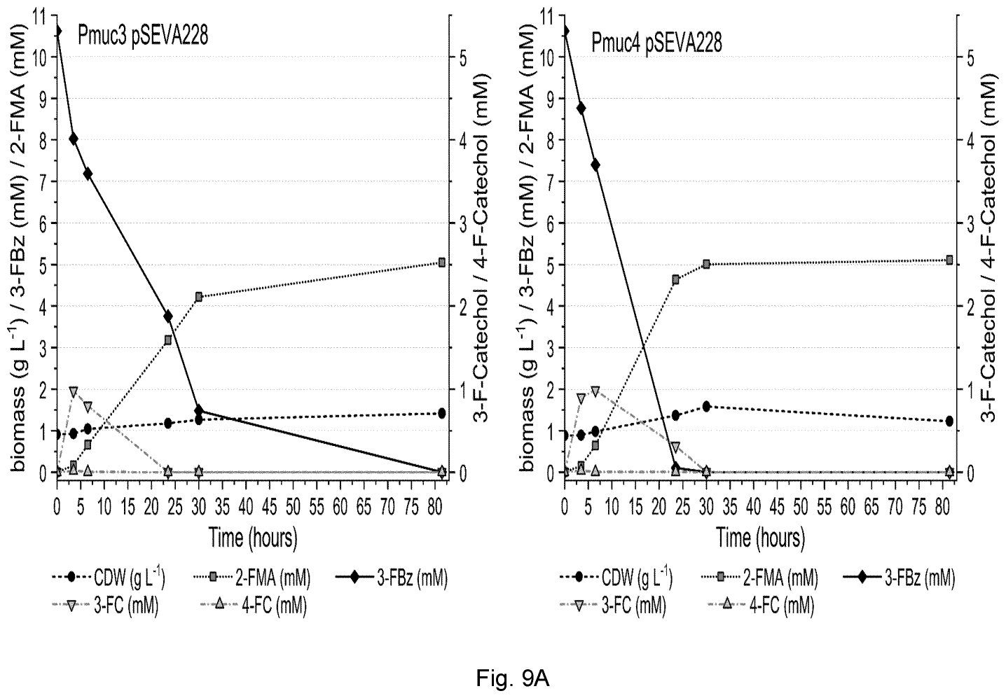

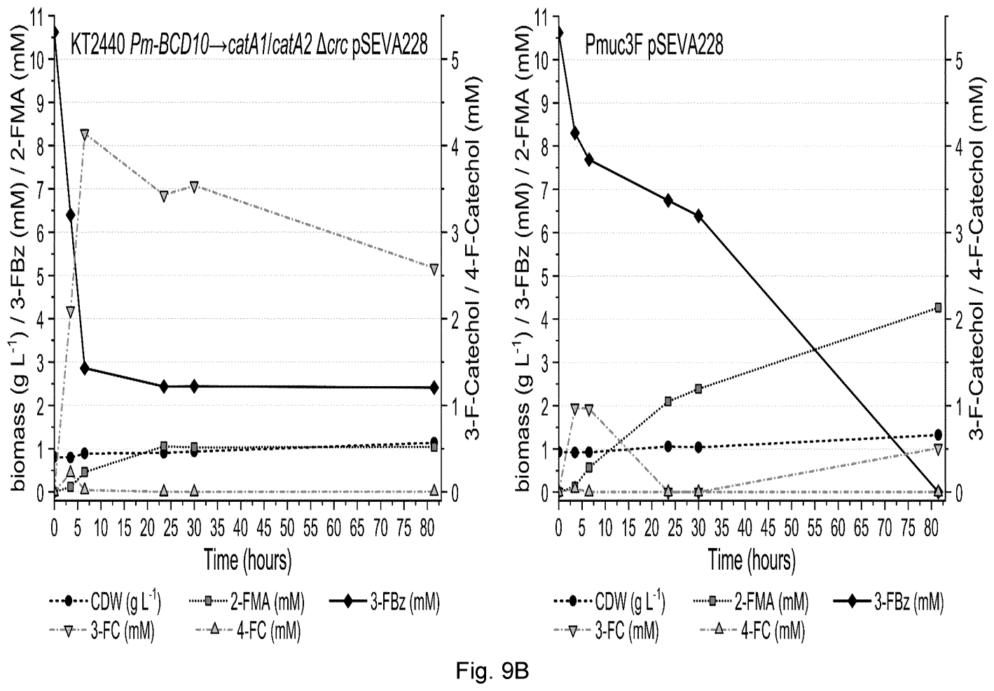

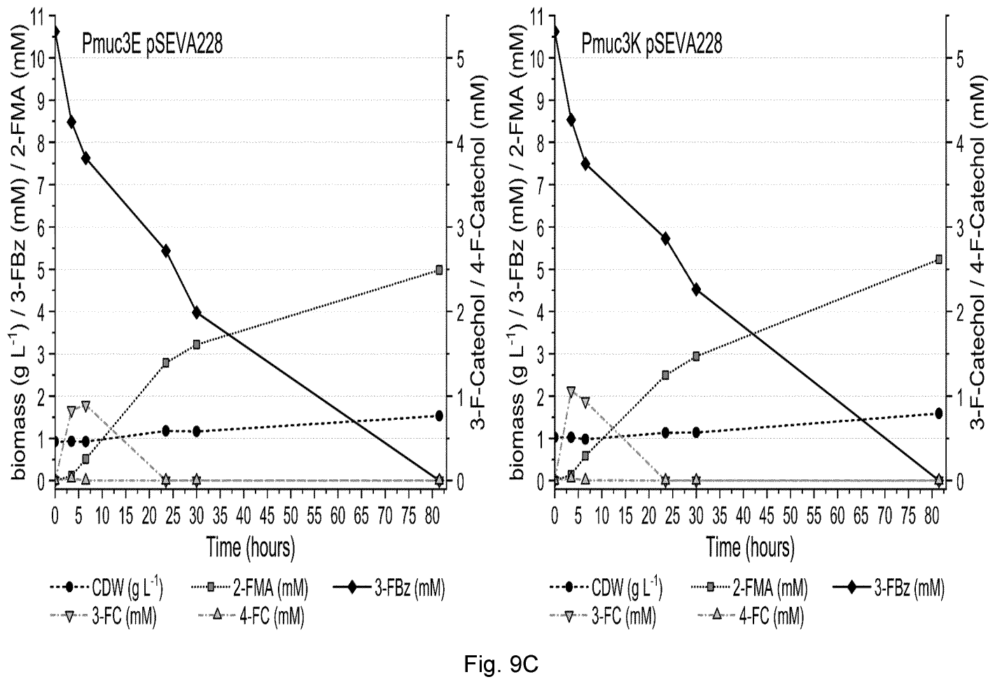

Each of these strains, Pmuc3, Pmuc4, P. putida Pm-BCD10-→catA/catA-II Δcrc, Pmuc3 Pm(ML1-17)-BCD10→benK (Pmuc3K), Pmuc3 Pm(ML1-17)-BCD10→benE-II (Pmuc3E), and Pmuc3 Pm(ML1-17)-BCD10→nicP-I (Pmuc3F) were transformed with pSEVA228 to provide enough XylS activator for the various genes under Pm control and tested for their performance in the conversion of 10 mM 3-FBz in shaken flasks with 30 mM glucose as the source of carbon and energy. The concentration changes in biomass (estimated cell dry weight—CDW), 3-FBz, 3-FC, 4-FC, and 2-FMA for each strain in the course of 80 h of culture are displayed in FIGS. 9A-C and 16.

All strains except P. putida Pm-BCD10→catA/catA-11 Δcrc completely consumed 10 mM 3-FBz within the course of the experiment and after an initial accumulation of ˜1 mM 3-FC within the first 5 h, likely due to a lack of catA(0) induction before the start of the fermentation, the only 3-FC detectable at the end of the fermentations was at a concentration of 2.6 mM 3-FC for P. putida Pm-BCD10→catA/catA-II Δcrc and 0.5 mM 3-FC for Pmuc3 Pm(ML-17)-BCD10→nicP-1 (Pmuc3F). All other Pmuc3 derivatives completely converted about 50% of the 3-FBz provided into 2-FMA. However, there were striking differences in the rates at which 3-FBz was consumed and 2-FMA was produced. The respective biomass-specific 3-FBz uptake rates qₛ and 2-FMA formation rates qₚ for each strain are listed in Table 4 and 5, below.

TABLE 4
Performance parameters for selected strains used in this study
in the bioconversion of 10 mM 3-FBz. To determine the biomass-specific
3-FBz uptake rates q_{S} and 2-FMA formation rates q_{P}, cells
were cultured in Erlenmeyer flasks filled with 10% (v/v)
de Bont minimal medium supplemented with 30 mM glucose and
10 mM 3-FBz. Maximum growth rates were determined in microtiter
plates in the identical medium.

| | | q_{S} | q_{P} |
|---|---|---|---|
| | μmax | [mmol | [mmol |
| Strain | [h⁻¹] | g_{CDW}⁻¹ h⁻¹] | g_{CDW}⁻¹ h⁻¹] |
| P. knackmussii | no growth | 0.090 ^{a} | 0.046 ^{b} |
| P. putida KT2440 | 0.13 | 0.162 ^{a} | 0.06 |
| Pmuc3 pSEVA228 | 0.28 | 0.226 | 0.14 ^{b} |
| Pmuc4 pSEVA228 | 0.30 | 0.398 | 0.199 ^{b} |
| Pm-BCD10→catA1/catA- | no growth | 1.409 ^{a} | 0.084 |
| II Δcrc pSEVA228 | | | |
| Pmuc3F pSEVA228 | 0.29 | 0.116 | 0.081 ^{b} |
| Pmuc3E pSEVA228 | 0.30 | 0.163 | 0.112 ^{b} |
| Pmuc3K pSEVA228 | 0.27 | 0.14 | 0.098 ^{b} |
| Ptac→benABC | 0.32 | 0.368 | 0.051 |
| Pm-BCD2→catA | | | |
| pSEVA228 | | | |
| Ptac→benABC | 0.39 | 0.105 | 0.053 ^{b} |
| Pm-BCD2→cat-II | | | |
| pSEVA228 | | | |
| Pm-BCD10→catA1/catA2 | 0.17 | 0.257 | 0.133 ^{b} |
| Ptac-BCD10→benA | | | |
| pSEVA228 | | | |
| Pmuc4vhb pSEVA228 | 0.24 | 0.311 | 0.167 ^{b} |
| Pmuc4tat-vhb pSEVA228 | 0.12 | 0.128 ^{a} | 0.061 ^{b} |
| Pmuc4c pSEVA228 | 0.19 | 0.650 ^{a} | 0.114 |
| Pmuc4g pSEVA228 | 0.15 | 0.596 ^{a} | 0.128 |

^{a} no complete consumption of 3-FBz
^{b} q_{P} represents about 50% of q_{S}

TABLE 5
Performance parameters for selected strains used in this study
in the bioconversion of 10 mM 3-FBz. To determine the biomass-specific
3-FBz uptake rates q_{S} and 2-FMA formation rates q_{P}, cells
were cultured in Erlenmeyer flasks filled with 10% (v/v) de Bont
minimal medium supplemented with 30 mM glucose and 10 mM 3-FBz.
Maximum growth rates (μₘₐₓ) were determined in microtiter
plates in the identical medium and values were calculated
using Gaussian process regression.

| | | q_{S} | q_{P} |
|---|---|---|---|
| | | [mmol | [mmol |
| Strain | μ^{max} [h⁻¹] | g_{CDW}⁻¹ h⁻¹] | g_{CDW}⁻¹ h⁻¹] |
| P. knackmussii | no growth | 0.090 ^{a} | 0.046 ^{b} |
| P. putida KT2440 | 0.13 | 0.162 ^{a} | 0.060 |
| Pmuc3 pSEVA228 | 0.28 | 1.105 | 0.507 ^{b} |
| Pmuc4 pSEVA228 | 0.13 | 0.994 | 0.344 ^{b, c} |
| Pm- | no growth | 1.409 ^{a} | 0.135 |
| BCD10→catA1/catA-II | | | |
| Δcrc pSEVA228 | | | |
| Pmuc3F pSEVA228 | 0.29 | 0.116 | 0.081 ^{b} |
| Pmuc3E pSEVA228 | 0.30 | 0.208 | 0.110 ^{b} |
| Pmuc3K pSEVA228 | 0.27 | 0.202 | 0.106 ^{b} |
| Ptac→benABC | 0.32 | 0.368 | 0.051 |
| Pm-BCD2→catA | | | |
| pSEVA228 | | | |
| Ptac→benABC | 0.39 | 0.172 | 0.067 ^{b} |
| Pm-BCD2→cat-II | | | |
| pSEVA228 | | | |
| Pm-BCD10→catA1/catA2 | 0.17 | 0.257 | 0.133 ^{b} |
| Ptac-BCD10→benA | | | |
| pSEVA228 | | | |
| Pmuc4vhb pSEVA228 | 0.24 | 0.311 | 0.167 ^{b} |
| Pmuc4tat-vhb pSEVA228 | 0.12 | 0.128 ^{a} | 0.061 ^{b} |
| Pmuc4c pSEVA228 | 0.19 | 0.406 ^{a} | 0.082 |
| Pmuc4g pSEVA228 | 0.15 | 0.384 ^{a} | 0.091 |
| Pmuc10 | 0.28 | 1.230 | 0.537 ^{b} |
| Pmuc11 | 0.15 | 1.771 ^{a} | 0.526 |
| Pmuc12 | 0.41 | 0.248 ^{a} | 0.128 ^{b} |
| Pmuc13 pSEVA228 | 0.29 | 1.216 | 0.460 ^{b} |
| Pmuc13 pSEVA228.2 | 0.25 | 0.930 | 0.385 ^{b} |
| Pmuc14 | 0.30 | 1.051 | 0.521 ^{b} |

^{a} no complete consumption of 3-FBz
^{b} no detectable fluorocatechol at the end of the fermentation
^{c} Pmuc4 showed a very long lag phase of about 30 h with low biochemical activity, followed by a fast conversion of 3-FBz and consumption of glucose

A direct comparison of qₛ and qₚ allows to draw conclusions about the balance of biochemical activities within the ortho-cleavage pathway acting on 3-FBz. With approximately 50% of the substrate channeled into the 2-FMA producing 1,2-dioxygenation route, the qₚ value should be half of the qₛ value if no significant accumulation of intermediates or by-product occurs. The deletion of crc in Pmuc4 resulted in an increase in productivity by about 1.5-fold compared to Pmuc3, suggesting an enhanced substrate uptake or increased BenD activity. The deletion of crc in Pmuc4 also resulted in a long lag phase of about 30 h after exposure to 3-FBz, in which the biomass-specific 3-FBz consumption rate, the 2-FMA production rate, and especially the glucose consumption rate was severely decreased. After this extended time of adaptation, all specific rates increased four to seven-fold so that about half of the supplied 3-FBz and glucose were consumed within the last three hours of fermentation. The deletion of the global carbon catabolite regulator Crc appears to create inherent instabilities in the metabolic state of Δcrc cells, which can result in either an immediate fast growth and 3-FBz utilization followed by metabolic inactivity as observed in strain Pm-BCD10→catA1/catA-II Δcrc pSEVA228, or extended phases of “dormancy” followed by very high metabolic activity as displayed by Pmuc4. It will require further investigation on the underlying mechanisms responsible for this drastic shift in physiology and suitable cultivation conditions that allow for a reliable utilization of the maximum productivities observed for these two strains. The individual expression of nicP-I, benE-II or benK resulted in a decrease in the 3-FBz uptake rate and thus 2-FMA formation, providing evidence for their contributing role in 3-FBz transport which was reduced due to the weak induction of the genes by Pm. A crc deletion without the replacement of the native P_{ben} promoter driving benABC expression caused a qₛ increase by about 3.5-fold compared to strain Pmuc3 Δcrc harboring the P_{tac}→benABC modification. However, this strong increase in 3-FBz uptake was not counteracted by a sufficient catechol 1,2-dioxygenase activity, consequently leading to a high accumulation of 3-FC. This result demonstrates the importance of a static control of benABC decoupled from the host's regulatory network.

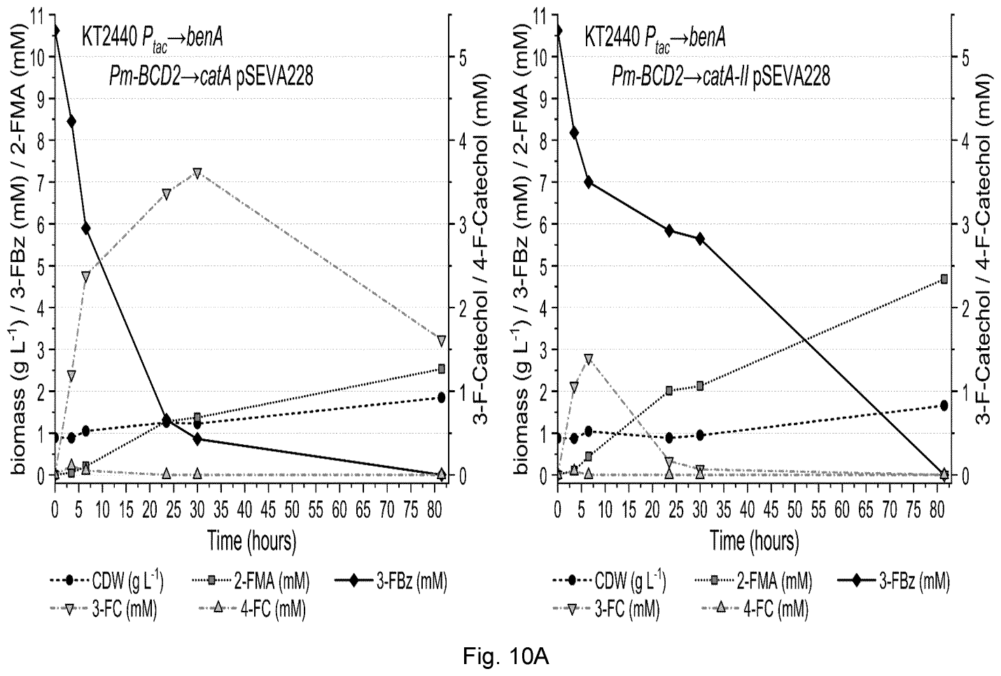

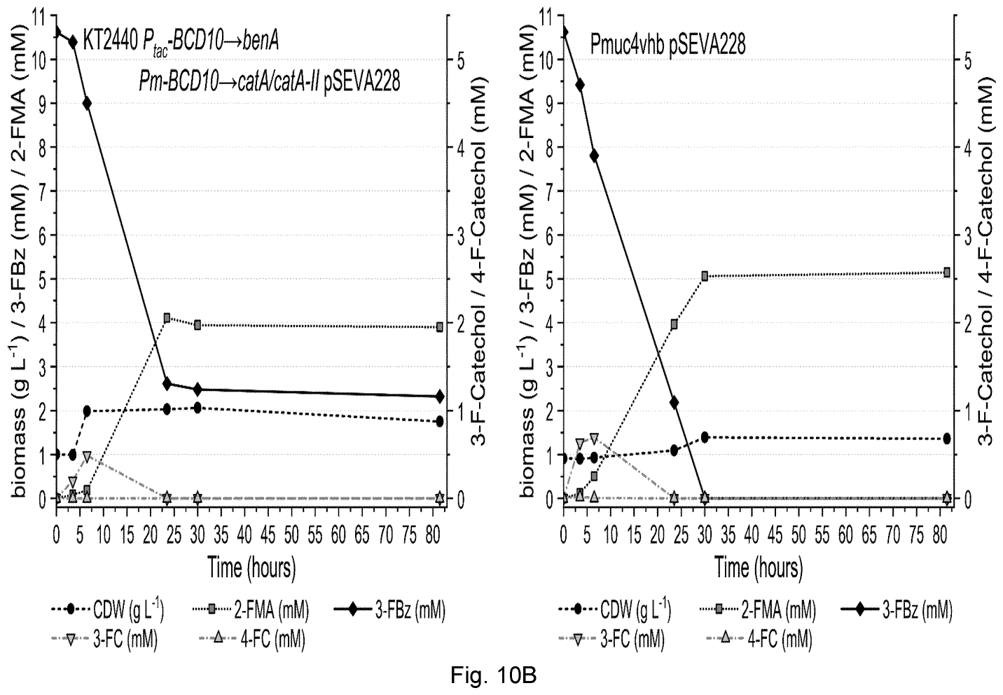

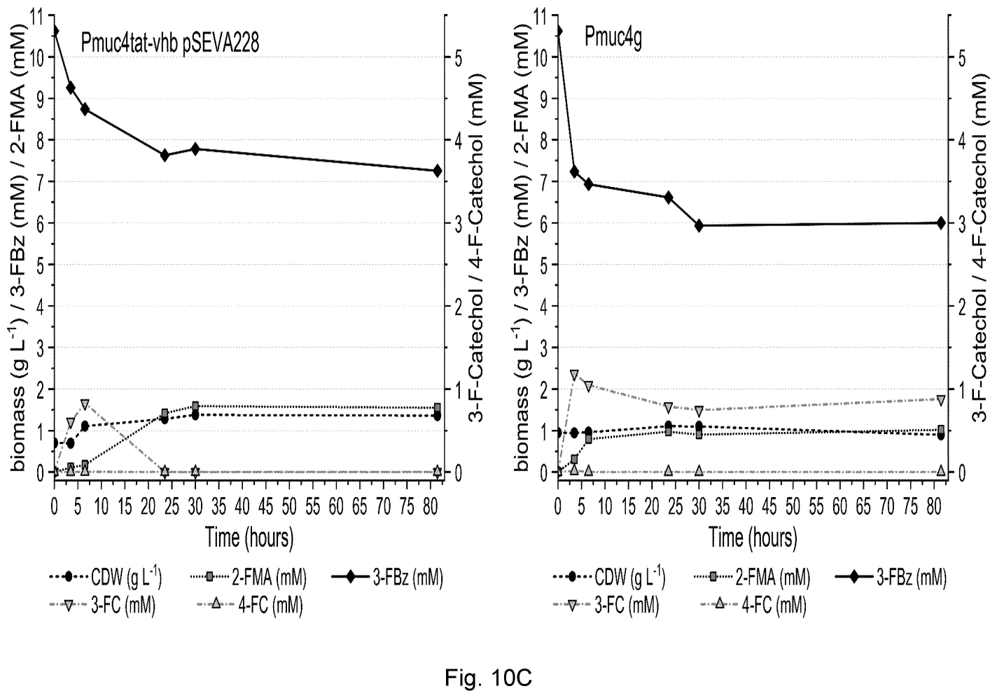

A further set of strains was created to further disclose the importance of each catA and catA-II via their individual expression with Pm and the stronger translational coupler BCD2, as well as to reveal the presence of a potential oxygen limitation (two reactions involved in the bioconversion consume molecular oxygen) via the introduction of the Vitreoscilla stercoraria vhb gene, encoding a bacterial hemoglobin. For the latter, vhb was inserted into the chromosomal crc locus under Pm-BCD10 control with (tat-vhb) and without (vhb) an N-terminal signal peptide for the P. putida type-II secretion system. Two last strains, the gene encoding xylS was integrated into the chromosome of Pmuc3 Δcrc under the control of BCD10 as well as the very strong P_{14g} promoter or P₁₄ with a reported strength of about 30% compared to P_{14g}, in an attempt to create an efficient plasmid-free bioconversion strain. The courses of fermentations with these six strains are shown in FIGS. 10A-C and their bioconversion performance parameters qₛ and qₚ are listed in Table 4 and 5, above.

The expressional control of only catA resulted in a significant decrease in 1,2-dioxygenase, apparent in the strong accumulation of 3-FC with a concentration of 3.6 mM. The expression control of catA-II by Pm-BCD2, however, provided sufficient 1,2-dioxygenase activity to enable a complete conversion of 3-FBz into 2-FMA at a maximum yield (50%), despite an initial 3-FC accumulation to 1.4 mM. Yet, the product formation rate qₚ was only less than 50% of the qₚ for Pmuc3, which has both catA and catA-II controlled by Pm-BCD10. Although no complete consumption of 3-FBz occurred with P. putida P_{tac}-BCD10→benABC Pm-BCD10→catA Pm-BCD10→catA-II, no accumulation of intermediates was observed and both qₚ and qₛ were significantly enhanced compared to strain Pmuc3 with benABC under its native translational control. The cytoplasmic expression of V. stercoraria vhb had no significant effect on the performance of Pmuc4, while tat-vhb expression impaired growth, even with glucose as the sole carbon compound (data not shown). The two Pmuc4 strains with xylS integrated into the genome upstream of catA under control of either P_{14g} (see FIGS. 10A-C) or P_{14c} (data not shown) showed almost identical metabolite profiles during the fermentations, with 30% (3.6 mM) of 3-FBz consumed within the first 6 h, 50% of which being converted into 0.9 mM 3-FC and 0.8 mM 2-FMA, and a complete stop of any further biochemical activities for the subsequent 74 h.

Based on the previous observations, a fourth set of strains was designed with all of the modifications that had a beneficial effect on 3-FBz uptake or bioconversion rates combined. These genomic modifications comprised P_{tac}-BCD10→benABC and the deletion of crc, as well as different types of regulatory sequences for catA and catA-II.

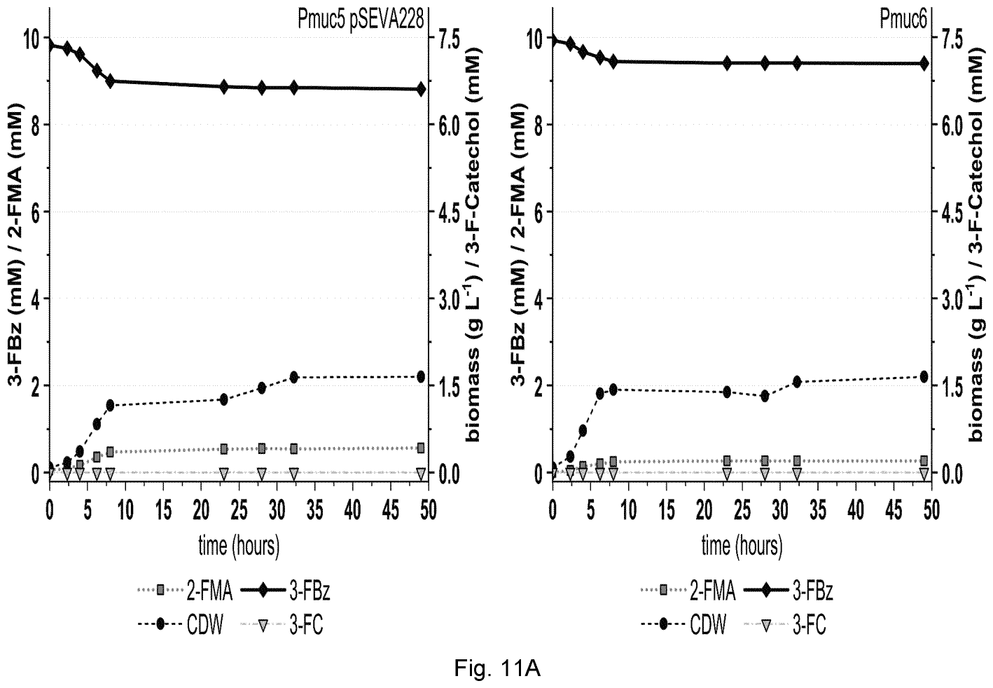

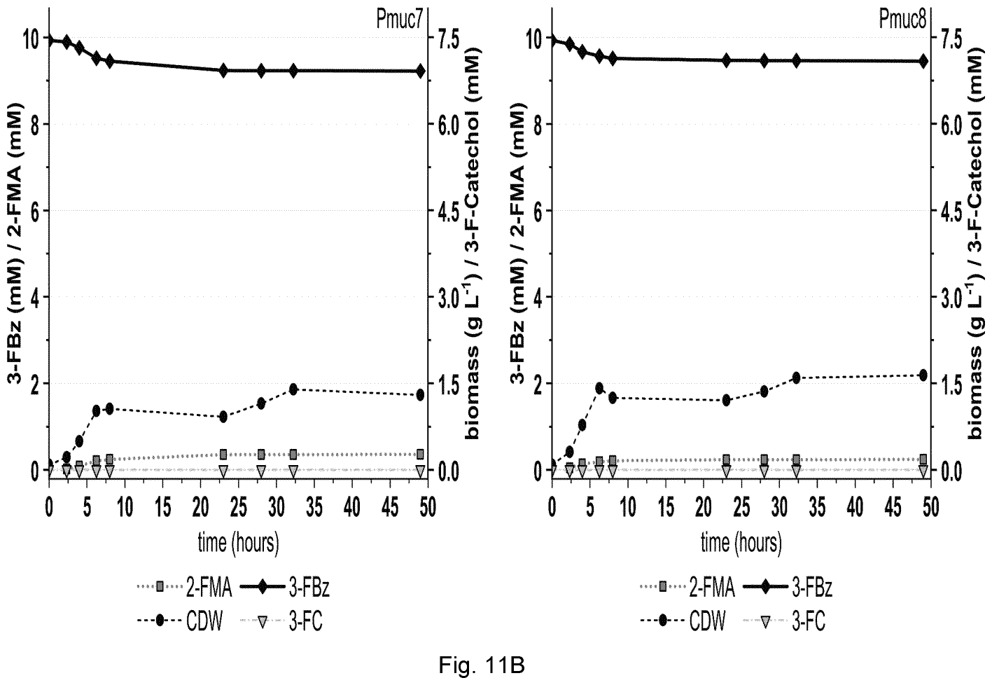

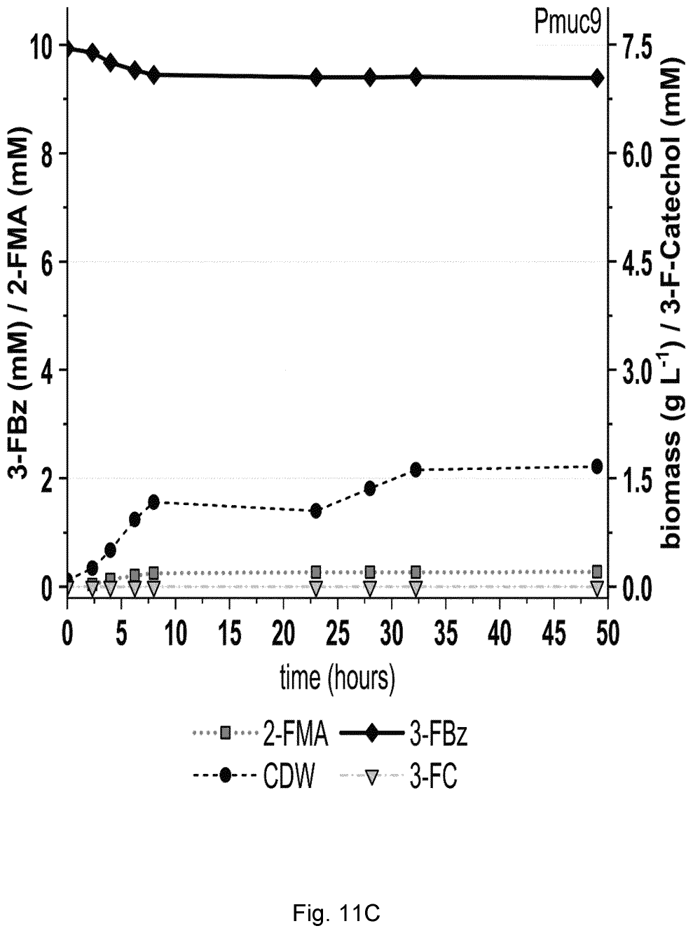

In Pmuc5, catA and catA-II were put under the control of Pm-BCD10 and Pm-BCD2, respectively, and XylS was provided from pSEVA228. In the plasmid-free strains Pmuc6, Pmuc7, Pmuc8, and Pmuc9, catA and catA-II were translationally controlled by BCD10 as well as the medium-weak, constitutive promoters J23115, J23108, J23114, and P_{14b}, respectively. The fermentation profiles of these five strains are shown in FIGS. 11A-C. Each strain consumed 3-FBz only within the first 8 hours of cultivation, and at low biomass-specific consumption rates. No formation of 3-FC or 4-FC could be observed. These results suggest that the specific combination of P_{tac}-BCD10→benABC and Δcrc are counterproductive for efficient 3-FBz utilization.

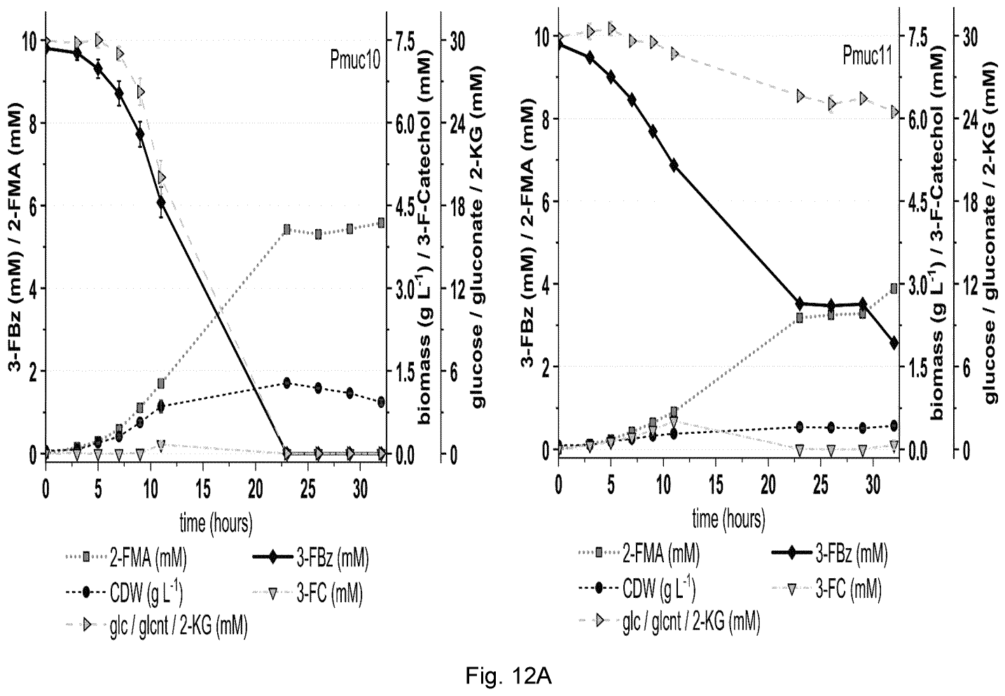

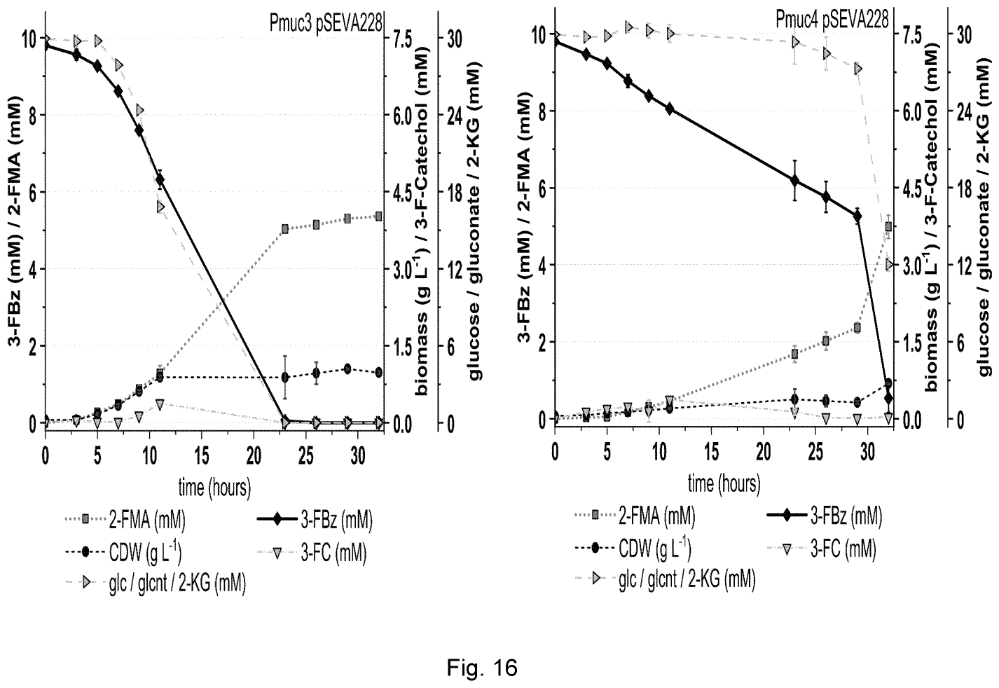

A fifth set of strains was created to characterize the specific effects of P_{tac}-BCD10→benABC and Δcrc, to enable an efficient bioconversion without the use of plasmids, and to establish the role of the transcription strength for catA-II. In the resulting strains Pmuc10, Pmuc11, Pmuc12, and Pmuc13, either only catA-II or both catA genes were put under the control of the constitutive promoter P_{14b} and BCD10. The combination of P_{tac}→benABC, P_{14b}-BCD10→catA, and P_{14b}-BCD10→catA-II in Pmuc10 enabled a complete conversion of all supplied 3-FBz at the maximum theoretical yield of 50% at the highest specific productivities observed (Table 4), without the necessity to provide the activator XylS on a plasmid. Furthermore, the constitutive expression of catA and catA-II decreased the initial accumulation of 3-FC as compared to Pmuc3, in which the catechol 1,2-dioxygenases were induced only at the start of the fermentation with the addition of 3-FBz. With both catA and catA-II constitutively expressed by P_{14b}, the additional deletion of crc (Pmuc11) or the introduction of BCD10 for benABC (Pmuc12) resulted in an incomplete consumption of 3-FBz at low specific rates. Thus, both genetic modifications brought about effects that are detrimental to the bioconversion. In strain Pmuc13, only catA-II was constitutively expressed, while catA was under the regulation of Pm. To achieve different induction strengths for catA, XylS was provided either with its wild-type sequence (on pSEVA228) or as variant XylS^{Thr45} which had been shown to cause a higher induction strength in response to 3-FBz compared to the wild-type variant by a factor of 2 [Ramos et al. (1990) and FIG. 6]. Pmuc13 demonstrated equal performances with pSEVA228 and pSEVA228.2, which were comparable to that of strain Pmuc3 (FIG. 16, FIG. 12B, and FIG. 12C). The additional increase in expression for catA-II did neither result in increased bioconversion productivity, nor resulted in a decrease of initial fluorocatechol accumulation. In Pmuc14, the xylS gene, under the transcriptional control of the strong promoter P_{14g}, was integrated into the chromosome upstream of catA-II. Both catA and catA-II were put under the control of Pm-BCD10, and benABC was regulated by P_{tac} and its native translation initiation sequence. Unlike with Pmuc4g and Pmuc3x, in which xylS was integrated upstream of catA, its integration in proximity to catA-II provided sufficient expression from the chromosomal Pm promoters to enable a complete conversion of 3-FBz at a rate comparable to Pmuc3, without the necessity of a plasmid.

It can be expected that the regulatory sequences successfully employed to control the expression of benABC, catA, and catA-II can be replaced with promoters and translation initiation sequences of comparable strength without disrupting the functionality of the pathway. Alternative sequences for P_{tac} include other medium-to-high strength promoters (FIG. 15), e.g., P_{14g}, P_{14f}, P₁₄ₑ, P_{14c}, P_{BG19}, P_{BG34} and P_{14d}, as characterized by Zobel et al. (2015), P_{EM7}, the Anderson promoters P_{J23119}, P_{J23101}, and P_{J23107}, as well as P_{JEc3}, P_{JEa3}, P_{JE1611}, and P_{JEa2}, as characterized by Elmore et al. (2020). Alternative sequences for catA and catA-II include other low-to-medium strength promoters (FIG. 15), e.g., the Anderson promoters P_{J23108} and P_{J23114}, promoter P_{14d} (Zobel et al., 2015), or the promoters PJ1611, PJEa2, and PJ1312 (Elmore et al., 2020). Alternative translation initiation sequences include any sequence that provides comparable translation initiation strength as the native benA 5′ UTR for benABC, or the bi-cistronic translational couplers BCD2 and BCD10 for catA and catA-II (see Table 1).

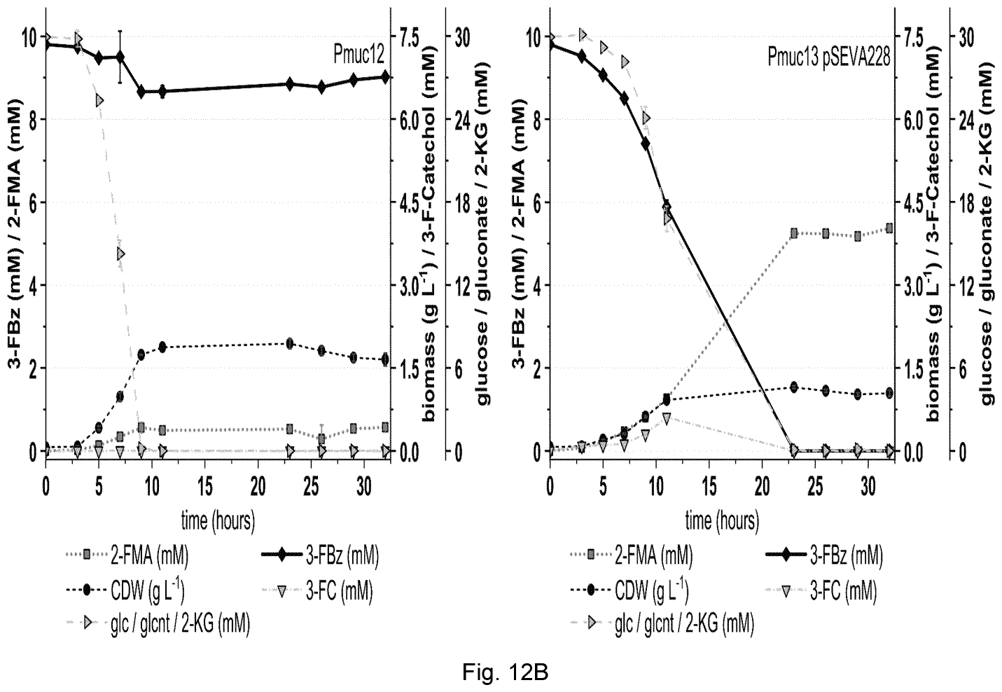

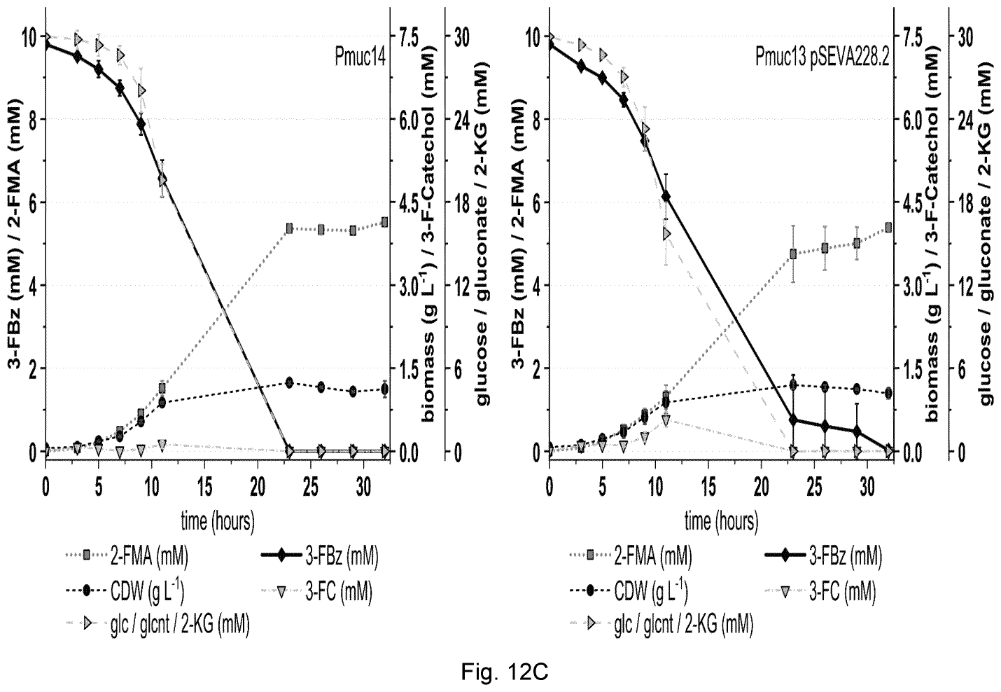

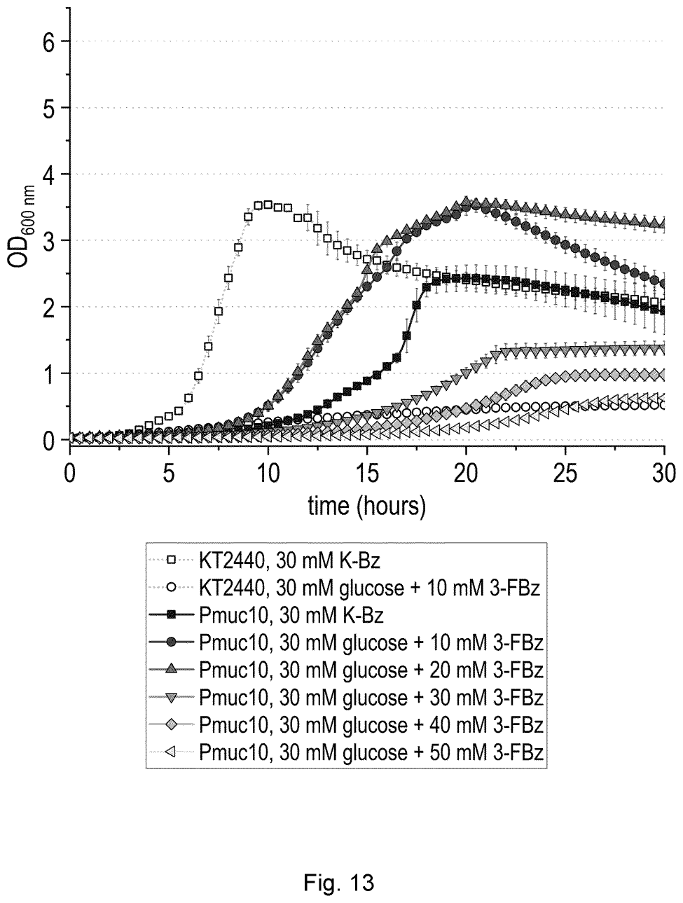

Pmuc10 was tested in microtiter plate cultivations regarding its ability to utilize benzoate as the sole carbon source, as well as the maximum 3-FBz concentrations tolerated by the strain in the presence of 30 mM glucose as the source of carbon and energy (FIG. 13). Up to a 3-FBz concentration of 20 mM, no toxic effects were observed. The strain was able to produce biomass up to the highest concentration of 50 mM 3-FBz, although at a strongly reduced rate and maximum biomass concentration. No coloration of the medium could be observed, indicating that the 3-FBz consumed was assimilated without the formation of fluorocatechol. Since the growth deficiencies observed in the growth rate were present from the beginning of the cultivation, 3-FBz itself seems to exhibit toxic effects on the cells. Nonetheless, the high tolerance of Pmuc10 to 3-FBz will facilitate an upscaling of the bioconversion process. Interestingly, the growth rate and biomass yield of Pmuc10 on benzoate were significantly reduced compared to wild-type P. putida KT2440. This suggests that the enzymes involved in the ortho-cleavage pathway require different relative expression levels when acting on halogenated or non-halogenated benzoates, and underlines the importance of combinatorial pathway balancing when toxic metabolites are involved.

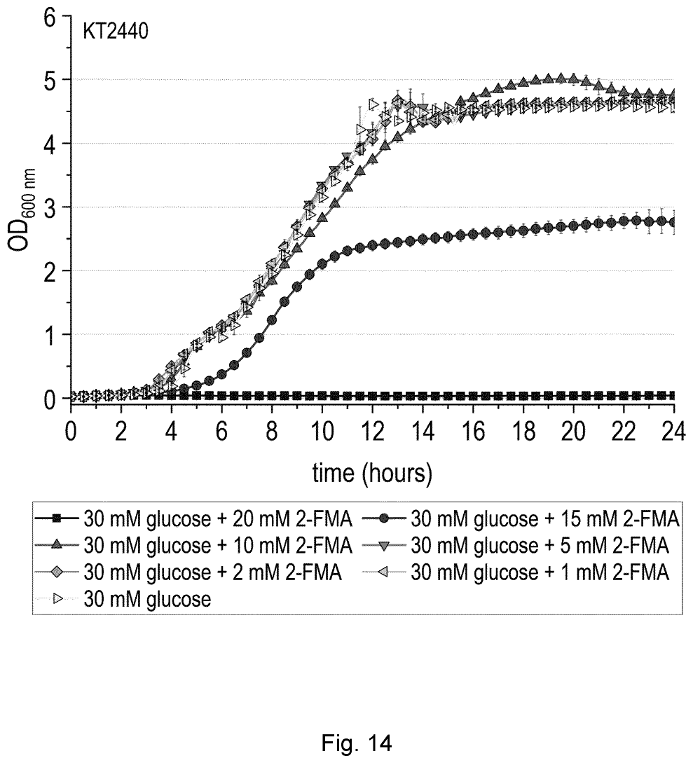

With the 2-FMA purified from fermentation broths, the toxicity of the product on the chosen bacterial platform P. putida was investigated in microtiter plate cultivations in minimal medium with 30 mM glucose and varying concentrations of the bioconversion product (FIG. 14). With up to 10 mM 2-FMA, P. putida KT2440 demonstrated a growth profile that was similar to cultures with glucose as the only carbon compound in the medium. At 15 mM 2-FMA, the growth rate and maximum biomass concentration were reduced by 50%, and at 20 mM 2-FMA, no growth could be detected. The tolerance of P. putida to 2-FMA can likely be further enhanced via adaptive laboratory evolution.

### Example 6—Wild-Type P. putida KT2440 Converts Fluoro-Substituted Benzoic Acids into Fluoro-Muconic Acid (Improved Method)

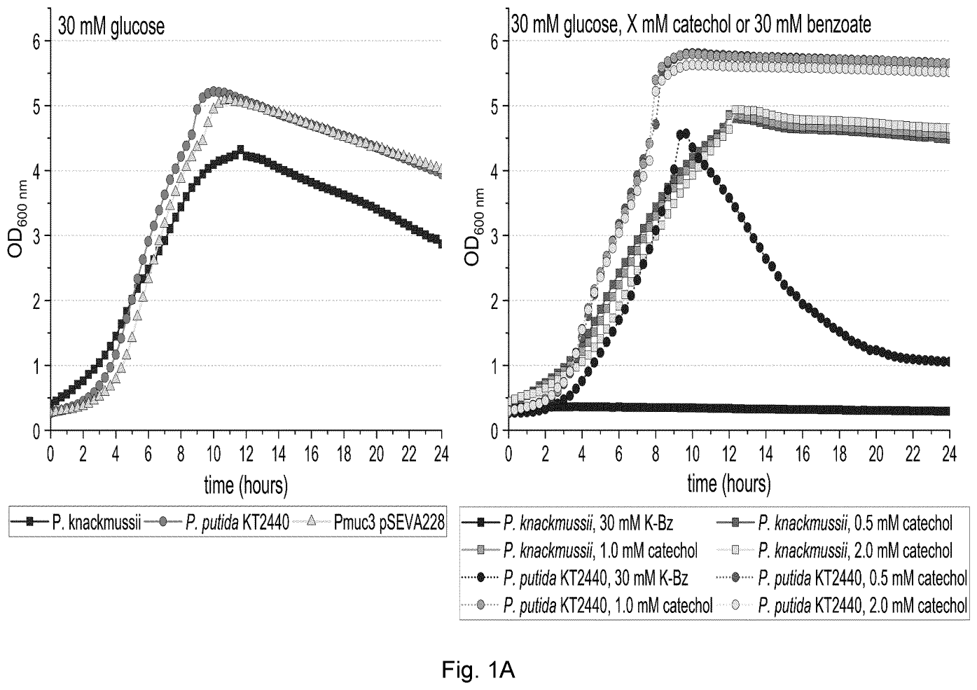

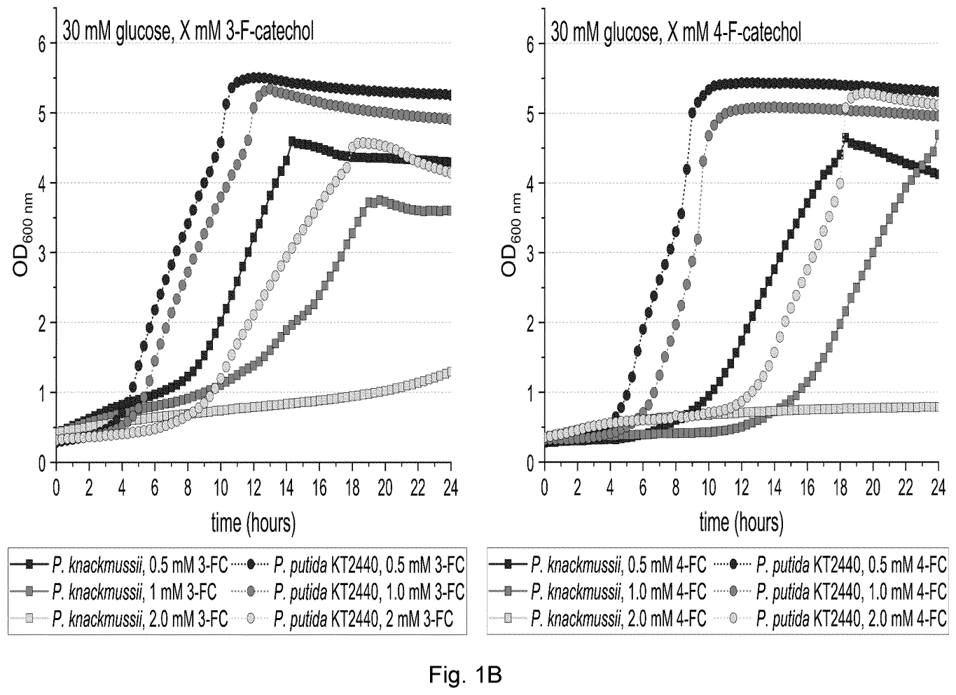

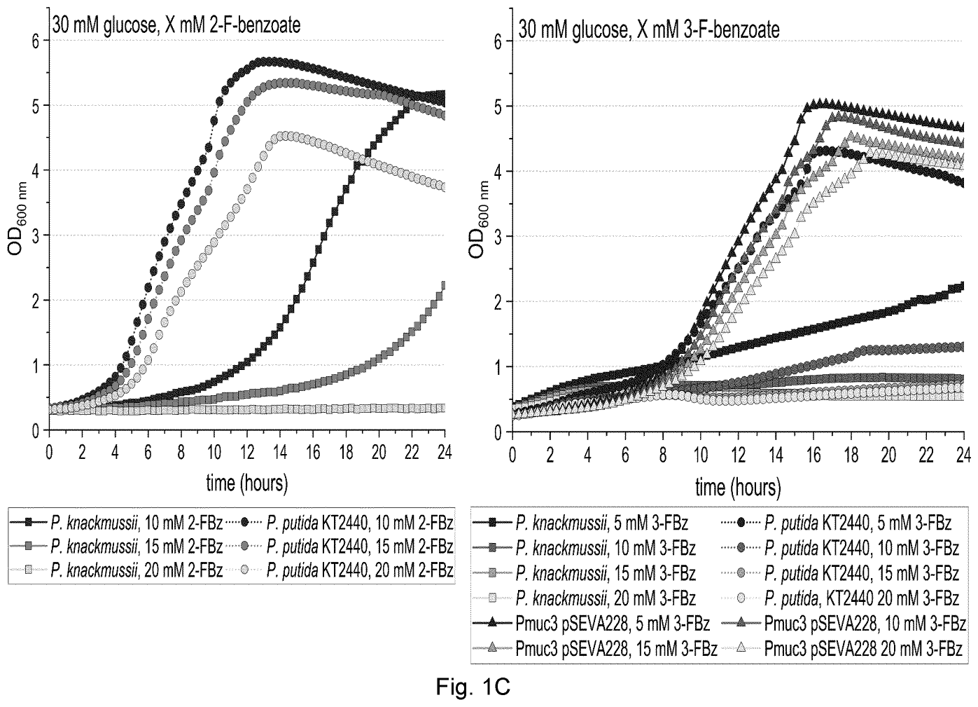

To examine the suitability of the ortho-cleavage pathway enzymes in P. putida for the bioconversion of 2- or 3-FBz to 2-FMA, both P. knackmussii and P. putida KT2440 were cultured in microtiter plates with minimal medium supplemented with 30 mM glucose (or 30 mM potassium benzoate) and varying concentrations of metabolites involved in the bioconversion process (FIGS. 1A-C). Maximum exponential growth rates (μₘₐₓ) observed, determined in the early exponential phase for each growth curve individually, are listed in Table 6, below. Using the updated method of Gaussian Process Regression calculate the maximum growth rates is more robust, more objective, and better reflects growth-inhibiting effects. Additional control experiments were also performed for this Example.

TABLE 6
Growth rates of selected strains used in this study. Cells
were precultured in LB medium supplemented. The experiment
was performed in 96-well microtiter plates with each well
containing 200 μl de Bont medium with 10 g L⁻¹ MOPS and
varying concentrations of carbon sources and metabolites
involved in the bioconversion process. Growth rates were
determined by performing an exponential regression to the
growth curves within the early exponential phase. Every
regression had a coefficient of determination (R²) of >0.998.

| | Maximum growth rate μₘₐₓ |
|---|---|
| Strain/media additives | (h⁻¹) |
| P. putida KT2440 | |
| 30 mM glucose | 0.86 |
| 30 mM K-benzoate | 0.62 |
| 30 mM glucose, 0.5 mM catechol | 0.69 |
| 30 mM glucose, 1.0 mM catechol | 0.71 |
| 30 mM glucose, 2.0 mM catechol | 0.68 |
| 30 mM glucose, 0.5 mM 3-F-catechol | 0.71 |
| 30 mM glucose, 1.0 mM 3-F-catechol | 0.68 |
| 30 mM glucose, 2.0 mM 3-F-catechol | 0.36 |
| 30 mM glucose, 5.0 mM 3-F-catechol | no growth |
| 30 mM glucose, 0.5 mM 4-F-catechol | 0.72 |
| 30 mM glucose, 1.0 mM 4-F-catechol | 0.52 |
| 30 mM glucose, 2.0 mM 4-F-catechol | 0.34 |
| 30 mM glucose, 5.0 mM 4-F-catechol | no growth |
| 30 mM glucose, 10 mM K-2-F-benzoate | 0.56 |
| 30 mM glucose, 15 mM K-2-F-benzoate | 0.52 |
| 30 mM glucose, 20 mM K-2-F-benzoate | 0.46 |
| 30 mM glucose, 5.0 mM K-3-F-benzoate | 0.25 |
| 30 mM glucose, 10 mM K-3-F-benzoate | 0.13 |
| 30 mM glucose, 15 mM K-3-F-benzoate | no growth |
| 30 mM glucose, 20 mM 2-FMA | no growth |
| 30 mM glucose, 15 mM 2-FMA | 0.66 |
| 30 mM glucose, 10 mM 2-FMA | 0.80 |
| 30 mM glucose, 5 mM 2-FMA | 0.86 |
| 30 mM glucose, 2 mM 2-FMA | 1.00 |
| 30 mM glucose, 1 mM 2-FMA | 0.86 |
| P. knackmussii | |
| 30 mM glucose | 0.35 |
| 30 mM K-benzoate | no growth |
| 30 mM glucose, 0.5 mM catechol | 0.36 |
| 30 mM glucose, 1.0 mM catechol | 0.33 |
| 30 mM glucose, 2.0 mM catechol | 0.29 |
| 30 mM glucose, 0.5 mM 3-F-catechol | 0.32 |
| 30 mM glucose, 1.0 mM 3-F-catechol | 0.31 |
| 30 mM glucose, 2.0 mM 3-F-catechol | 0.12 |
| 30 mM glucose, 0.5 mM 4-F-catechol | 0.31 |
| 30 mM glucose, 1.0 mM 4-F-catechol | 0.26 |
| 30 mM glucose, 2.0 mM 4-F-catechol | 0.17 |
| 30 mM glucose, 10 mM K-2-F-benzoate | 0.25 |
| 30 mM glucose, 15 mM K-2-F-benzoate | 0.19 |
| 30 mM glucose, 20 mM K-2-F-benzoate | no growth |
| 30 mM glucose, 5.0 mM K-3-F-benzoate | 0.04 |
| 30 mM glucose, 10 mM K-3-F-benzoate | no growth |
| 30 mM glucose, 15 mM K-3-F-benzoate | no growth |
| 30 mM glucose, 20 mM K-3-F-benzoate | no growth |
| Pmuc3 pSEVA228 | |
| 30 mM glucose | 0.84 |
| 30 mM K-benzoate, 0.5 mM 3-mBz | 0.16 |
| 30 mM glucose, 0.5 mM catechol | 0.62 |
| 30 mM glucose, 1.0 mM catechol | 0.74 |
| 30 mM glucose, 2.0 mM catechol | 0.81 |
| 30 mM glucose, 0.5 mM 3-F-catechol | 0.74 |
| 30 mM glucose, 1.0 mM 3-F-catechol | 0.72 |
| 30 mM glucose, 2.0 mM 3-F-catechol | 0.22 |
| 30 mM glucose, 0.5 mM 4-F-catechol | 0.55 |
| 30 mM glucose, 1.0 mM 4-F-catechol | 0.50 |
| 30 mM glucose, 2.0 mM 4-F-catechol | 0.28 |
| 30 mM glucose, 5.0 mM K-3-F-benzoate | 0.36 |
| 30 mM glucose, 10 mM K-3-F-benzoate | 0.33 |
| 30 mM glucose, 15 mM K-3-F-benzoate | 0.32 |
| 30 mM glucose, 20 mM K-3-F-benzoate | 0.30 |
| Pmuc10 | |
| 30 mM glucose | 0.97 |
| 30 mM K-benzoate | 0.53 |
| 30 mM glucose, 2.0 mM 3-F-catechol | no growth |
| 30 mM glucose, 2.0 mM 4-F-catechol | 0.49 |
| 30 mM glucose, 2.0 mM 4-F-catechol | no growth |
| 30 mM glucose, 10 mM K-3-F-benzoate | 0.45 |
| 30 mM glucose, 20 mM K-3-F-benzoate | 0.47 |
| 30 mM glucose, 30 mM K-3-F-benzoate | 0.27 |
| 30 mM glucose, 40 mM K-3-F-benzoate | 0.24 |
| 30 mM glucose, 50 mM K-3-F-benzoate | 0.20 |
| 30 mM glucose, 20 mM 2-FMA | no growth |
| 30 mM glucose, 15 mM 2-FMA | 0.47 |
| 30 mM glucose, 10 mM 2-FMA | 0.98 |
| 30 mM glucose, 5 mM 2-FMA | 1.08 |
| 30 mM glucose, 2 mM 2-FMA | 1.20 |
| 30 mM glucose, 1 mM 2-FMA | 1.24 |

Within the course of 50 h, no growth was detectable for P. knackmussii on benzoate as the sole source of carbon, while P. putida grew with a μₘₐₓ of 0.62 h⁻¹. With glucose as the primary source of carbon and energy, P. putida grew with a maximum rate μₘₐₓ of 0.86 h⁻¹, which is 2.5 times higher than that of P. knackmussii (μₘₐₓ 0.35 h⁻¹). In the presence of 2-FBz, both species demonstrated some degree of sensitivity to the compound, which became more prominent with increasing concentrations. However, while P. putida still grew with 53% of its maximum rate on glucose and without any noticeable effect on the duration of the lag phase in the presence of 20 mM 2-FBz, P. knackmussii was unable to grow at all within the course of the experiment (50 h) and showed significantly increased lag phases at 10 mM and 15 mM of 2-FBz. At 15 mM 2-FBz, P. knackmussii grew with 54% of the μₘₐₓ observed on only glucose. The detrimental impact of the second bioconversion substrate 3-FBz on the growth of both species was more severe. P. putida was able to grow in the presence of maximum 10 mM 3-FBz with a substantially reduced μₘₐₓ value. Even at 5 mM, the growth of P. knackmussii was almost completely impaired and no growth was observable at concentrations of 10 mM.

Depending on the position of the fluoro-substitution on the benzoate derivative, the three 1,2-dihydroxybenzenes pyrocatechol (here referred to as catechol), 3-FC, and 4-FC are produced as intermediary metabolites. Only a little toxic effect was observed in the presence of up to 2 mM catechol on both P. putida and P. knackmussii. The physiological response of the two species to the two fluorinated 1,2-dihydroxybenzene derivatives, 3-FC and 4-FC, was more pronounced. While both 3-FC and 3-FC affected the cells' growth rate in a similar manner, the presence of 4-FC had a more prominent effect on the duration of lag phases. However, in both cases, P. putida demonstrated a higher resistance to the toxic effects of both 3-FC and 4-FC than P. knackmussii.

After establishing the physiological response of both P. knackmussii and P. putida to the chemical compounds involved in the bioconversion of FBz to 2-FMA, the two species were subjected to a fermentation experiment in shaken flasks containing synthetic de Bont medium with 10 g L⁻¹ MOPS at pH 7.0, and supplemented with 30 mM glucose as well as 10 mM of 2-FBz or 3-FBz (FIGS. 2A-C). P. putida completely consumed 2-FBz within 20 h with a substrate uptake rate qₛ of 1.07 mmol g_{CDW}⁻¹ h⁻¹. 2-FMA was the only compound detectable after substrate depletion, at a concentration of 1.34 mM and produced at a product formation rate qₚ of 0.06 mmol g_{CDW}⁻¹ h⁻¹. Neither 3-FC nor 4-FC were detected at any point in time. After an initial consumption of 2-FBz with a qₛ of 0.81 mmol g_{CDW}⁻¹ h⁻¹ for about 20 h, P. knackmussii stopped taking up any further substrate (final concentration of 3.2 mM). Out of the 6 mM substrate consumed, about 1 mM 2-FMA was produced at a product formation rate qₚ of 0.03 mmol g_{CDW}⁻¹ h⁻¹. Furthermore, ccMA could be detected in the culture supernatant at a maximum concentration of 1.55 mM after 30 h, which continued to be consumed slowly but not completely until the end of the fermentation (50 h). For both bacterial species, the amount of 2-FMA produced represents 14% of the 3-FBz consumed (product yield coefficient Y_{SP}=0.14 mol mol-1).

Exposed to 3-FBz, P. putida consumed the substrate up until a time between 7 and 20 h with a qₛ of 0.98 mmol g_{CDW}⁻¹ after which the 3-FBz concentration in the culture supernatant remained constant. Within the first 7 h, all 3-FBz consumed was converted into 3-FC until it reached a concentration of 1 mM. In the course of the next 13 h, 40% of 3-FC in the supernatant was converted into 2-FMA, combined with a fraction of additionally consumed 3-FBz at a combined product formation rate qₚ of 0.06 mmol g_{CDW}⁻¹ h⁻¹. At the end of the fermentation, 3.48 mM 3-FBz had been converted into 1.38 mM 2-FMA, corresponding to a product yield Y_{SP} of 0.41 mol mol-1, as well as 0.52 mM 3-FC. The combined concentrations of metabolites constituting the 1,2-dioxygenation route (2-FMA and 3-FC) add up to 1.90 mM, representing 55% of the consumed 3-FBz. Considering its previously established toxicity, the accumulation of 3-FC was identified as a major bottleneck for an efficient and prolonged bioconversion process in P. putida. Also with P. knackmussii, 3-FBz consumption occurred only within the first 7-20 h of the fermentation, with a qₛ of 1.01 mmol g_{CDW}⁻¹ h⁻¹. After an initial increase of 3-FC up to 0.94 mM in the culture supernatant, the catechol derivative was completely re-consumed until 20 h. From a total concentration of 2.93 mM 3-FBz consumed, P. knackmussii produced 1.64 mM 2-FMA [corresponding to 56% of the substrate (Y_{SP}: 0.41 mol mol-1)] with a rate qₚ of 0.09 mM g_{CDW}⁻¹ h⁻¹. In none of the conditions tested significant amounts of 4-FC could be detected in the culture supernatants. The enzyme machinery performing the di-oxygenation on fluorinated catechol derivatives that both species are equipped with thus seems to demonstrate a higher efficiency towards 4-FC compared to 3-FC.

With the asymmetrical use of the non-productive 1,2-dioxygenation route when using 2-FBz as substrate for the bioconversion, almost 90% of fluorine gets lost, in contrast to about 50% with 3-FBz as substrate. The 3-F-substituted benzoate consequently represents the most suitable precursor compound to produce 2-FMA. A faster growth rate, higher biomass yield on glucose, and a higher resistance to toxic effects of pathway intermediates make P. putida stand out as the bacterial platform better suited to perform this bioconversion.

### REFERENCES

Bagdasarian, M., R. Lurz, B. Rückert, F. C. H. Franklin, M. M. Bagdasarian, J. Frey and K. N. Timmis (1981). “Specific purpose plasmid cloning vectors. II. Broad host range, high copy number, RSF1010-derived vectors, and a host-vector system for gene cloning in Pseudomonas.” Gene 16(1-3): 237-247.
Bakke, I., L. Berg, T. E. V. Aune, T. Brautaset, H. Sletta, A. Tondervik and S. Valla (2009). “Random mutagenesis of the Pm promoter as a powerful strategy for improvement of recombinant-gene expression.” Applied and Environmental Microbiology 75(7): 2002-2011.
Cavaleiro, A. M., S. H. Kim, S. Seppälä, M. T. Nielsen and M. H. H. Nørholm (2015). “Accurate DNA assembly and genome engineering with optimized uracil excision cloning.” ACS Synthetic Biology 4(9): 1042-1046.
Choi, K. H., A. Kumar and H. P. Schweizer (2006). “A 10-min method for preparation of highly electrocompetent Pseudomonas aeruginosa cells: application for DNA fragment transfer between chromosomes and plasmid transformation.” Journal of Microbiological Methods 64(3): 391-397.
de Boer, H. A., L. J. Comstock and M. Vasser (1983). “The tac promoter: a functional hybrid derived from the trp and lac promoters.” Proceedings of the National Academy of Sciences USA 80(1): 21-25.
Elder, R. T. (1983). “Cloning techniques.” BioScience 33(11): 721-722.
Elmore, J. R., Dexter, G. N., Francis, R., Riley, L., Huenemann, J., Baldino, H., Guss, A. M., Egbert, R., 2020. The SAGE genetic toolkit enables highly efficient, iterative site-specific genome engineering in bacteria. bioRxiv. 2020.06.28.176339
Genee, H. J., M. T. Bonde, F. O. Bagger, J. B. Jespersen, M. O. A. Sommer, R. Wernersson and L. R. Olsen (2015). “Software-supported USER cloning strategies for site-directed mutagenesis and DNA Assembly.” ACS Synthetic Biology 4(3): 342-349.
Hall, B. G., H. Acar, A. Nandipati and M. Barlow (2014). “Growth rates made easy.” Molecular Biology and Evolution 31(1): 232-238.
Hartmans, S., J. P. Smits, M. J. van der Werf, F. Volkering and J. A. de Bont (1989). “Metabolism of styrene oxide and 2-phenylethanol in the styrene-degrading Xanthobacter strain 124X.” Applied Environmental Microbiology 55(11): 2850-2855.
Kohlstedt, M., S. Starck, N. Barton, J. Stolzenberger, M. Selzer, K. Mehlmann, R. Schneider, D. Pleissner, J. Rinkel, J. S. Dickschat, J. Venus, J. B. J. H. van Duuren and C. Wittmann (2018). “From lignin to nylon: Cascaded chemical and biochemical conversion using metabolically engineered Pseudomonas putida.” Metabolic Engineering 47: 279-293.
Martinez-Garćia, E., T. Aparicio, A. Goni-Moreno, S. Fraile and V. De Lorenzo (2015). “SEVA 2.0: An update of the Standard European Vector Architecture for de-/re-construction of bacterial functionalities.” Nucleic Acids Research 43.
Martinez-Garcia, E., T. Aparicio, A. Goni-Moreno, S. Fraile and V. de Lorenzo (2014). “SEVA 2.0: an update of the Standard European Vector Architecture for de-/re-construction of bacterial functionalities.” Nucleic Acids Research 43(D1): D1183-D1189.
Mutalik, V. K., J. C. Guimaraes, G. Cambray, C. Lam, M. J. Christoffersen, Q.-a. Mai, A. B. Tran, M. Paull, J. D. Keasling, A. P. Arkin and D. Endy (2013). “Precise and reliable gene expression via standard transcription and translation initiation elements.” Nature Methods 10: 354-360.
Pérez-Pantoja, D., J. Kim, R. Silva-Rocha and V. de Lorenzo (2015). “The differential response of the Pben promoter of Pseudomonas putida mt-2 to BenR and XylS prevents metabolic conflicts in m-xylene biodegradation.” Environmental Microbiology 17(1): 64-75.
Platt, R., C. Drescher, S. K. Park and G. J. Phillips (2000). “Genetic system for reversible integration of DNA constructs and lacZ gene fusions into the Escherichia coli chromosome.” Plasmid 43(1): 12-23.
Putker, F., R. Tommassen-van Boxtel, M. Stork, J. J. Rodriguez-Herva, M. Koster and J. Tommassen (2013). “The type II secretion system (Xcp) of Pseudomonas putida is active and involved in the secretion of phosphatases.” Environmental Microbiology 15(10): 2658-2671.
Ramos, J. L., Michan, C., Rojo, F., Dwyer, D., & Timmis, K. N. (1990). Signal-regulator interactions. Genetic analysis of the effector binding site of xylS, the benzoate-activated positive regulator of Pseudomonas TOL plasmids meta-cleavage pathway operon. Journal of Molecular Biology, 211, 373.
Reiner, A. M. (1972). Metabolism of aromatic-compounds in bacteria—purification and properties of catechol-forming enzyme, 3,5-cyclohexadiene-1,2-diol-1-carboxylic acid (nad+) oxidoreductase (decarboxylating). Journal of Biological Chemistry, 247(16), 4960-+.
Schmidt, E. and H. J. Knackmuss (1984). “Production of cis,cis-muconate from benzoate and 2-fluoro-cis,cis-muconate from 3-fluorobenzoate by 3-chlorobenzoate degrading bacteria.” Applied Microbiology and Biotechnology 20(5): 351-355.
Stolz, A., H.-J. Busse and P. Kämpfer (2007). “Pseudomonas knackmussii sp. nov.” International Journal of Systematic and Evolutionary Microbiology 57(3): 572-576.
Swain, P. S., Stevenson, K., Leary, A., Montano-Gutierrez, L. F., Clark, I. B. N., Vogel, J., Pilizota, T., 2016. Inferring time derivatives including cell growth rates using Gaussian processes. Nature Communications. 7, 13766.
Vardon, D. R., Rorrer, N. A., Salvachúa, D., Settle, A. E., Johnson, C. W., Menart, M. J., Cleveland, N. S., Ciesielski, P. N., Steirer, K. X., Dorgan, J. R., & Beckham, G. T. (2016). cis,cis-Muconic acid: separation and catalysis to bio-adipic acid for nylon-6,6 polymerization. Green Chem., 18(11), 3397-3413. https://doi.org/10.1039/C5GC02844B
Volke, D. C., L. Friis, N. T. Wirth, J. Turlin and P. I. Nikel (2020). “Synthetic control of plasmid replication enables target- and self-curing of vectors and expedites genome engineering of Pseudomonas putida.” Metabolic Engineering Communications 10: e00126.
Wirth, N. T., E. Kozaeva and P. I. Nikel (2019). “Accelerated genome engineering of Pseudomonas putida by I-SceI-mediated recombination and CRISPR-Cas9 counterselection.” Microbial Biotechnology 0(0).
Wojcieszyńska D, Guzik U, Grén, I, Perkosz M, Hupert-Kocurek K. Induction of aromatic ring: cleavage dioxygenases in Stenotrophomonas maltophilia strain KB2 in cometabolic systems. World J Microbiol Biotechnol. 2011; 27(4):805-811. doi:10.1007/s11274-010-0520-6
Worsey, M. J. and P. A. Williams (1975). “Metabolism of toluene and xylenes by Pseudomonas putida (arvilla) mt-2: evidence for a new function of the TOL plasmid.” Journal of Bacteriology 124(1): 7-13.
Yamaguchi M, Fujisawa H. Purification and characterization of an oxygenase component in benzoate 1,2-dioxygenase system from Pseudomonas arvilla C-1. J Biol Chem. 1980; 255(11):5058-5063.
Zobel, S., I. Benedetti, L. Eisenbach, V. De Lorenzo, N. Wierckx and L. M. Blank (2015). “Tn7-Based Device for Calibrated Heterologous Gene Expression in Pseudomonas putida.” ACS Synthetic Biology 4: 1341-1351.

### Sequences

| SEQ ID | | |
|---|---|---|
| NO: | Description | Organism and optionally accession number |
| 1 | BenA (protein) | P. putida KT2440 (UniProt accession number Q88I40) |
| 2 | BenB (protein) | P. putida KT2440 (UniProt accession number Q88I39) |
| 3 | BenC (protein) | P. putida KT2440 (UniProt accession number Q88I38) |
| 4 | benA (DNA) | P. putida KT2440 (RefSeq NP_745305.1) |
| 5 | benB (DNA) | P. putida KT2440 (RefSeq NP_745306.1) |
| 6 | benC (DNA) | P. putida KT2440 (RefSeq NP_745307.1) |
| 7 | BenD (protein) | P. putida KT2440 (UniProt accession number Q88I37) |
| 8 | benD (DNA) | P. putida KT2440 (RefSeq NP_745308.1) |
| 9 | CatA-I (protein) | P. putida KT2440 (UniProt accession number Q88GK8) |
| 10 | catA-I (DNA) | P. putida KT2440 (RefSeq NP_745846.1) |
| 11 | CatA-II (protein) | P. putida KT2440 (UniProt accession number Q88I35) |
| 12 | catA-II (DNA) | P. putida KT2440 (RefSeq NP_745310.1) |
| 13 | Pben promoter | P. putida KT2440 |
| | (DNA) | |
| 14 | Ptac promoter | Artificial sequence |
| | (DNA) | |
| 15 | Pm promoter | P. putida mt-2 |
| | (DNA) | |
| 16 | BCD10 (DNA) | Artificial sequence |
| 17 | BCD6 (DNA) | Artificial sequence |
| 18 | Pm promoter | Artificial sequence |
| | variant ML1-17 | |
| | (DNA) | |
| 19 | BCD5 (DNA) | Artificial sequence |
| 20 | xylS (DNA) | P. putida mt-2 (RefSeq NP_542858.1) |
| 21 | XylS (protein) | P. putida mt-2 (UniProt accession number P07859) |
| 22 | BCD2 (DNA) | Artificial sequence |
| 23 | BCD1 (DNA) | Artificial sequence |
| 24 | benA translation | P. putida KT2440 |
| | initiation region | |
| | (DNA) | |
| 25 | Regulatory | P. putida KT2440 |
| | sequence | |
| | upstream of | |
| | benD (DNA) | |
| 26 | VHb (protein) | Vitreoscilla stercoraria (UniProt accession number: |
| | | P04252) |
| 27 | VHb (DNA) | Codon-optimized for P. putida, (originally NCBI |
| | | accession number M27061) |
| 28 | Gluconate 2- | P. putida KT2440 (UniProt accession number Q88HH4) |
| | dehydrogenase | |
| | (Gad) gamma | |
| | subunit (protein) | |
| 29 | Gluconate 2- | P. putida KT2440 (Gene Symbol PP_3383) |
| | dehydrogenase | |
| | (Gad) gamma | |
| | subunit (DNA) | |
| 30 | Gluconate 2- | P. putida KT2440 (UniProt accession number Q88HH5) |
| | dehydrogenase | |
| | (Gad) | |
| | flavoprotein | |
| | subunit (protein) | |
| 31 | Gluconate 2- | P. putida KT2440 (Gene Symbol PP_3383) |
| | dehydrogenase | |
| | (Gad) | |
| | flavoprotein | |
| | subunit (DNA) | |
| 32 | Gluconate 2- | P. putida KT2440 (UniProt accession number Q88HH4) |
| | dehydrogenase | |
| | (Gad) | |
| | cytochrome c | |
| | subunit (protein) | |
| 33 | Gluconate 2- | P. putida KT2440 (Gene Symbol PP_3382) |
| | dehydrogenase | |
| | (Gad) | |
| | cytochrome c | |
| | subunit (DNA) | |
| 34 | Glucose | P. putida KT2440 (UniProt accession number Q88MX4) |
| | dehydrogenase | |
| | (Gcd) (protein) | |
| 35 | Glucose | P. putida KT2440 (Gene Symbol PP_1444) |
| | dehydrogenase | |
| | (gcd) (DNA) | |
| 36 | Crc (protein) | P. putida KT2440 (UniProt accession number Q88C91) |
| 37 | crc (DNA) | P. putida KT2440 (Gene Symbol PP_5292) |
| 38 | BenK (protein) | P. putida KT2440 (UniProt accession number Q88I36) |
| 39 | benK (DNA) | P. putida KT2440 (Gene Symbol PP_3165) |
| 40 | BenE-II (protein) | P. putida KT2440 (UniProt accession number Q88I34) |
| 41 | benE-II (DNA) | P. putida KT2440 (Gene Symbol PP_3167) |
| 42 | NicP-I (protein) | P. putida KT2440 (UniProt accession number Q88I33) |
| 43 | nicP-I (DNA) | P. putida KT2440 (Gene Symbol PP_3168) |
| 44 | BCD11 (DNA) | Artificial sequence |
| 45 | BCD7 (DNA) | Artificial sequence |
| 46 | J23108 | Artificial sequence |
| | promoter (DNA) | |
| 47 | J23114 | Artificial sequence |
| | promoter (DNA) | |
| 48 | J23115 | Artificial sequence |
| | promoter (DNA) | |
| 49 | P_{14b} promoter | Artificial sequence |
| | (DNA) | |
| 50 | P_{14g} promoter | Artificial sequence |
| 51 | xylS.2 (DNA) | Artificial sequence, with nucleotide 134(G) in xylS |
| | | replaced by C |
| 52 | XylS.2 (protein) | Artificial sequence, with amino acid 45(Arg) in XylS |
| | | replaced by Thr |
| 53 | P_{14f} promoter | Artificial sequence |
| 54 | P₁₄ₑ promoter | Artificial sequence |
| 55 | P_{14d} promoter | Artificial sequence |
| 56 | BG19 promoter | Artificial sequence |
| 57 | BG34 promoter | Artificial sequence |
| 58 | P_{14c} promoter | Artificial sequence |
| 59 | P_{EM7} promoter | Artificial sequence |
| 60 | J23119 | Artificial sequence |
| | promoter | |
| 61 | J23101 | Artificial sequence |
| | promoter | |
| 62 | J23107 | Artificial sequence |
| | promoter | |
| 63 | JEc3 promoter | Artificial sequence |
| 64 | JEa3 promoter | Artificial sequence |
| 65 | JE1611 | Artificial sequence |
| | promoter | |
| 66 | JEa2 promoter | Artificial sequence |
| 67 | J1312 promoter | Artificial sequence |
| 68 | BCD22 | Artificial sequence |
| 69 | BCD16 | Artificial sequence |
| 70 | BCD23 | Artificial sequence |
| 71 | BCD8 | Artificial sequence |
| 72 | BCD21 | Artificial sequence |
| 73 | MCD18 | Artificial sequence |
| 74 | MCD8 | Artificial sequence |
| 75 | MCD14 | Artificial sequence |
| 76 | MCD15 | Artificial sequence |
| 77 | MCD22 | Artificial sequence |
| 78 | BCD5 | Artificial sequence |
| 79 | BCD9 | Artificial sequence |
| 80 | BCD12 | Artificial sequence |
| 81 | BCD13 | Artificial sequence |
| 82 | BCD19 | Artificial sequence |
| 83 | BCD18 | Artificial sequence |
| 84 | BCD20 | Artificial sequence |

### Item List

1. A bacterial cell capable of producing 2-fluoro-cis,cis-muconate and optionally derivatives thereof, in the presence of 3-fluorobenzoate or a salt thereof, said bacterial cell expressing:
   a native or heterologous benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
   a native or heterologous benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
   a first, and optionally a second, native or heterologous catechol 1,2-dioxygenases (EC: 1.13.11.1), wherein the first and second catechol 1,2-dioxygenases are each capable of converting 3-fluorocatechol to 2-fluoro-cis,cis-muconate,
wherein:
   the expression of said benzoate 1,2-dioxygenase is under the control of a medium-strong constitutive promoter, whereby the benzoate 1,2-dioxygenase is constitutively overexpressed;
   the expression of said first catechol 1,2-dioxygenase is under the control of a first expression module comprising a first promoter, said first expression module further comprising a first strong translation initiation sequence such as a first strong translational coupler, and
   the expression of said second catechol 1,2-dioxygenase is under the control of a second expression module comprising a second promoter, said second expression module further comprising a second strong translational initiation sequence such as a second strong translational coupler,
   whereby the bacterial cell is capable of converting 3-fluorobenzoate to 2-fluoro-cis,cis-muconate.
2. The bacterial cell according item 1, wherein the first and/or the second promoter is a medium-to-weak constitutive promoter, whereby the catechol 1,2-dioxygenase is constitutively expressed.
3. The bacterial cell according to item 2, wherein the medium-to-weak constitutive promoter is P_{14b} (SEQ ID NO: 49) or a variant thereof having at least 80% homology, such as at least 85%, such as at least 90%, such as at least 95% homology or identity thereto.
4. The bacterial cell according to item 1, wherein the first and/or the second promoter is a first and/or a second inducible promoter, optionally wherein the first and/or second promoter is coupled with a sequence encoding an associated activator protein.
5. The bacterial cell according to any one of the preceding items, wherein the first and/or the second strong translation initiation sequence has similar translation initiation strength as BCD2 (SEQ ID NO: 22) or BCD10 (SEQ ID NO: 16).
6. A bacterial cell capable of producing 2-fluoro-cis,cis-muconate and optionally derivatives thereof, in the presence of 3-fluorobenzoate or a salt thereof, said bacterial cell expressing:
   a native or heterologous benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
   a native or heterologous benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
   a first native or heterologous catechol 1,2-dioxygenase (EC: 1.13.11.1), wherein the catechol 1,2-dioxygenase is capable of converting 3-fluorocatechol to 2-fluoro-cis,cis-muconate,
wherein:
   the expression of said benzoate 1,2-dioxygenase is under the control of a medium-strong constitutive promoter, whereby the benzoate 1,2-dioxygenase is constitutively overexpressed; and
   the expression of said first catechol 1,2-dioxygenase is under the control of a first expression module comprising a first inducible promoter coupled with a sequence encoding an associated activator protein, said first expression module further comprising a first strong translational coupler, whereby said first catechol 1,2-dioxygenase is expressed in response to 3-fluorobenzoate,
whereby the bacterial cell is capable of converting 3-fluorobenzoate to 2-fluoro-cis,cis-muconate.
7. A bacterial cell capable of producing 2-fluoro-cis,cis-muconate and optionally derivatives thereof, in the presence of 3-fluorobenzoate or a salt thereof, said bacterial cell expressing:
   a native or heterologous benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
   a native or heterologous benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
   a first and a second native or heterologous catechol 1,2-dioxygenases (EC: 1.13.11.1), wherein the first and second catechol 1,2-dioxygenases are each capable of converting 3-fluorocatechol to 2-fluoro-cis,cis-muconate,
wherein:
   the expression of said benzoate 1,2-dioxygenase is under the control of a medium-strong constitutive promoter, whereby the benzoate 1,2-dioxygenase is constitutively overexpressed;
   the expression of said first catechol 1,2-dioxygenase is under the control of a first expression module comprising a first inducible promoter coupled with a sequence encoding an associated activator protein, said first expression module further comprising a first strong translational coupler,
   whereby said first catechol 1,2-dioxygenase is expressed in response to 3-fluorobenzoate; and
   the expression of said second catechol 1,2-dioxygenase is under the control of a second expression module comprising a second inducible promoter, said second expression module further comprising a second strong translational coupler whereby said second catechol 1,2-dioxygenase is expressed in response to 3-fluorobenzoate,
whereby the bacterial cell is capable of converting 3-fluorobenzoate to 2-fluoro-cis,cis-muconate.
8. The bacterial cell according to any one of the preceding items, wherein the benzoate 1,2-dioxygenase is BenABC or a functional variant thereof having at least 80% homology, such as at least 85%, such as at least 90%, such as at least 95% homology or identity thereto, consisting of BenA (SEQ ID NO: 1) or a functional variant thereof having at least 80% homology, such as at least 85%, such as at least 90%, such as at least 95% homology or identity thereto, BenB (SEQ ID NO: 2) or a functional variant thereof having at least 80% homology, such as at least 85%, such as at least 90%, such as at least 95% homology or identity thereto, and BenC (SEQ ID NO: 3) or a functional variant thereof having at least 80% homology, such as at least 85%, such as at least 90%, such as at least 95% homology or identity thereto.
9. The bacterial cell according to any one of the preceding items, wherein the medium-strong constitutive promoter is P_{tac} (SEQ ID NO: 15), or a variant thereof having at least 80% homology, such as at least 85%, such as at least 90%, such as at least 95% homology or identity thereto.
10. The bacterial cell according to any one of the preceding items, wherein the benzoate-1,2-dihydrodiol dehydrogenase is BenD (SEQ ID NO: 7) or a functional variant thereof having at least 80% homology, such as at least 85%, such as at least 90%, such as at least 95% homology or identity thereto.
11. The bacterial cell according to any one of the preceding items, wherein the first catechol 1,2-dioxygenase is CatA-1 (SEQ ID NO: 9) or a functional variant thereof having at least 80% homology, such as at least 85%, such as at least 90%, such as at least 95% homology or identity thereto, and/or the second catechol 1,2-dioxygenase is CatA-II (SEQ ID NO: 11) or a functional variant thereof having at least 80% homology, such as at least 85%, such as at least 90%, such as at least 95% homology or identity thereto.
12. The bacterial cell according to any one of the preceding items, wherein the first inducible promoter of said first expression module is the Pm variant ML1-17 (SEQ ID NO: 18) or a variant thereof having at least 80% homology, such as at least 85%, such as at least 90%, such as at least 95% homology or identity thereto, the associated activator protein is XylS (SEQ ID NO: 21) or a functional variant thereof having at least 80% homology, such as at least 85%, such as at least 90%, such as at least 95% homology or identity thereto, and the first strong translational coupler is BCD2 (SEQ ID NO: 22) or BCD10 (SEQ ID NO: 16), or a variant thereof having at least 80% homology, such as at least 85%, such as at least 90%, such as at least 95% homology or identity thereto, preferably the first strong translational coupler is BCD10 (SEQ ID NO: 16) or a variant thereof having at least 80% homology, such as at least 85%, such as at least 90%, such as at least 95% homology or identity thereto.
13. The bacterial cell according to any one of the preceding items, wherein the second inducible promoter of said second expression module is Pm (SEQ ID NO: 15) or a variant thereof having at least 80% homology, such as at least 85%, such as at least 90%, such as at least 95% homology or identity thereto, and the second strong translational coupler is BCD10 (SEQ ID NO: 16) or a variant thereof having at least 80% homology, such as at least 85%, such as at least 90%, such as at least 95% homology or identity thereto.
14. The bacterial cell according to any one of the preceding items, wherein the bacterial cell is a non-pathogenic organism.
15. The bacterial cell according to any one of the preceding items, wherein the bacterial cell is a bacterial cell of the Pseudomonas genus, the Burkholderia genus, the Escherichia genus, the Rhodococcus genus, the Bacillus genus or the Vibrio genus.
16. The bacterial cell according to any one of the preceding items, wherein the bacterial cell is a bacterial cell selected from Pseudomonas putida, Pseudomonas knackmussi, Pseudomonas fluorescens, Pseudomonas taiwanesis, Pseudomonas syringae, Pseudomonas stutzeri, Pseudomonas oleovorans, Pseudomonas mendocina, Burkholderia fungorum, Escherichia coli, Rhodococcus rhodochrous, Bacillus subtilis, Bacillus cereus, Bacillus megaterium, Vibrio natriegens and, preferably the bacterial cell is a Pseudomonas putida cell, such as a Pseudomonas putida KT2440 cell.
17. The bacterial cell according to any one of the preceding items, wherein the bacterial cell is Pseudomonas putida KT2440.
18. The bacterial cell according to any one of the preceding items, wherein the bacterial cell is capable of producing 2-fluoro-cis,cis-muconate with a titer of at least 500 mg/L, such as at least 750 mg/L, such as at least 1 g/L, such as at least 1.5 g/L, such as at least 2 g/L, such as at least 2.5 g/L, such as at least 3 g/L, such as at least 3.5 g/L, such as at least 4 g/L, such as at least 4.5 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
19. The bacterial cell according to any one of the preceding items, further comprising modifications resulting in increased growth performance on sugars.
20. The bacterial cell according to any one of the preceding items, further comprising mutation(s) or deletion(s) of the gamma subunit of Gad (SEQ ID NO: 28), the flavoprotein subunit of Gad (SEQ ID NO: 30), the cytochrome c subunit of Gad (SEQ ID NO: 32) and/or Gcd (SEQ ID NO: 34).
21. The bacterial cell according to any one of the preceding items, further comprising mutation or deletion of the catabolite repression control protein Crc (SEQ ID NO: 36).
22. The bacterial cell according to any one of the preceding items, wherein one or more of the genes encoding the benzoate 1,2-dioxygenase, the medium-strong constitutive promoter, the benzoate-1,2-dihydrodiol dehydrogenase, the first catechol 1,2-dioxygenase, the second catechol 1,2-dioxygenase, the first expression module and/or the second expression module are codon-optimized for the bacterial cell.
23. The bacterial cell according to any one of the preceding items, wherein one or more of the genes encoding the benzoate 1,2-dioxygenase, the medium-strong constitutive promoter, the benzoate-1,2-dihydrodiol dehydrogenase, the first catechol 1,2-dioxygenase, the second catechol 1,2-dioxygenase, the first expression module and/or the second expression module are present in multiple copy number.
24. The bacterial cell according to any one of the preceding items, wherein one or more of the genes encoding the benzoate 1,2-dioxygenase, the medium-strong constitutive promoter, the benzoate-1,2-dihydrodiol dehydrogenase, the first catechol 1,2-dioxygenase, the second catechol 1,2-dioxygenase, the first expression module and/or the second expression module are integrated in the genome of the bacterial cell.
25. The bacterial cell according to any one of the preceding items, wherein one or more of the genes encoding the benzoate 1,2-dioxygenase, the medium-strong constitutive promoter, the benzoate-1,2-dihydrodiol dehydrogenase, the first catechol 1,2-dioxygenase, the second catechol 1,2-dioxygenase, the first expression module and/or the second expression module are expressed from a vector such as a plasmid.
26. A method of producing 2-fluoro-cis,cis-muconate, and optionally derivatives thereof, in a bacterial cell, said method comprising the steps of providing a bacterial cell and incubating said bacterial cell in a medium in the presence of 3-fluorobenzoate or a salt thereof, said bacterial cell expressing:
   a native or heterologous benzoate 1,2-dioxygenase (EC: 1.14.12.10);
   a native or heterologous benzoate-1,2-dihydrodiol dehydrogenase (EC: 1.3.1.25); and
   a first, and optionally a second, native or heterologous catechol 1,2-dioxygenase (EC: 1.13.11.1),
wherein:
   the expression of said benzoate 1,2-dioxygenase is under the control of a medium-strong constitutive promoter;
   the expression of said first catechol 1,2-dioxygenase is under the control of a first expression module comprising a first promoter, said first expression module further comprising a first strong translation initiation sequence such as a first strong translational coupler; and
   the expression of said second catechol 1,2-dioxygenase is under the control of a second expression module comprising a second promoter, said second expression module further comprising a second strong translational initiation sequence such as a second strong translational coupler.
27. The method according to item 26, wherein the first and/or the second promoter is a medium-to-weak constitutive promoter.
28. The method according to item 27, wherein the medium-to-weak constitutive promoter is P_{14b} (SEQ ID NO: 49) or a variant thereof having at least 80% homology, such as at least 85%, such as at least 90%, such as at least 95% homology or identity thereto.
29. The method according to item 26, wherein the first and/or the second promoter is a first and/or a second inducible promoter, optionally wherein the first and/or second promoter is coupled with a sequence encoding an associated activator protein.
30. The method according to any one of items 26 to 29, wherein the first and/or the second strong translation initiation sequence has similar translation initiation strength as BCD2 (SEQ ID NO: 22) or BCD10 (SEQ ID NO: 16).
31. A method of producing 2-fluoro-cis,cis-muconate, and optionally derivatives thereof, in a bacterial cell, said method comprising the steps of providing a bacterial cell and incubating said bacterial cell in a medium in the presence of 3-fluorobenzoate or a salt thereof, said bacterial cell expressing:
   a native or heterologous benzoate 1,2-dioxygenase (EC: 1.14.12.10);
   a native or heterologous benzoate-1,2-dihydrodiol dehydrogenase (EC: 1.3.1.25); and
   a first native or heterologous catechol 1,2-dioxygenase (EC: 1.13.11.1),
wherein:
   the expression of said benzoate 1,2-dioxygenase is under the control of a medium-strong constitutive promoter;
   the expression of said first catechol 1,2-dioxygenase is under the control of a first expression module comprising a first inducible promoter coupled with a sequence encoding an associated activator protein, said first expression module further comprising a first strong translational coupler.
32. A method of producing 2-fluoro-cis,cis-muconate, and optionally derivatives thereof, in a bacterial cell, said method comprising the steps of providing a bacterial cell and incubating said bacterial cell in a medium in the presence of 3-fluorobenzoate or a salt thereof, said bacterial cell expressing:
   a native or heterologous benzoate 1,2-dioxygenase (EC: 1.14.12.10);
   a native or heterologous benzoate-1,2-dihydrodiol dehydrogenase (EC: 1.3.1.25); and
   a first and a second native or heterologous catechol 1,2-dioxygenases (EC: 1.13.11.1),
wherein:
   the expression of said benzoate 1,2-dioxygenase is under the control of a medium-strong constitutive promoter;
   the expression of said first catechol 1,2-dioxygenase is under the control of a first expression module comprising a first inducible promoter coupled with a sequence encoding an associated activator protein, said first expression module further comprising a first strong translational coupler; and
   the expression of said second catechol 1,2-dioxygenase is under the control of a second expression module comprising a second inducible promoter, said second expression module further comprising a second strong translational coupler.
33. The method according to any one of items 26 to 32, wherein the benzoate 1,2-dioxygenase is BenABC or a functional variant thereof having at least 80% homology, such as at least 85%, such as at least 90%, such as at least 95% homology or identity thereto, consisting of BenA (SEQ ID NO: 1) or a functional variant thereof having at least 80% homology, such as at least 85%, such as at least 90%, such as at least 95% homology or identity thereto, BenB (SEQ ID NO: 2) or a functional variant thereof having at least 80% homology, such as at least 85%, such as at least 90%, such as at least 95% homology or identity thereto, and BenC (SEQ ID NO: 3) or a functional variant thereof having at least 80% homology, such as at least 85%, such as at least 90%, such as at least 95% homology or identity thereto.
34. The method according to any one of items 26 to 33, wherein the medium-strong constitutive promoter is Pₜac (SEQ ID NO: 15), or a variant thereof having at least 80% homology, such as at least 85%, such as at least 90%, such as at least 95% homology or identity thereto.
35. The method according to any one of items 26 to 34, wherein the benzoate-1,2-dihydrodiol dehydrogenase is BenD (SEQ ID NO: 7) or a functional variant thereof having at least 80% homology, such as at least 85%, such as at least 90%, such as at least 95% homology or identity thereto.
36. The method according to any one of items 26 to 35, wherein the first catechol 1,2-dioxygenase is CatA-1 (SEQ ID NO: 9) or a functional variant thereof having at least 80% homology, such as at least 85%, such as at least 90%, such as at least 95% homology or identity thereto, and/or the second catechol 1,2-dioxygenase is CatA-II (SEQ ID NO: 11) or a functional variant thereof having at least 80% homology, such as at least 85%, such as at least 90%, such as at least 95% homology or identity thereto.
37. The method according to any one of items 26 to 36, wherein the first inducible promoter of said first expression module is the Pm variant ML1-17 (SEQ ID NO: 18) or a variant thereof having at least 80% homology, such as at least 85%, such as at least 90%, such as at least 95% homology or identity thereto, and the associated activator protein is XylS (SEQ ID NO: 21) or a functional variant thereof having at least 80% homology, such as at least 85%, such as at least 90%, such as at least 95% homology or identity thereto, and the first strong translational coupler is BCD2 (SEQ ID NO: 22) or BCD10 (SEQ ID NO: 16), or a variant thereof having at least 80% homology, such as at least 85%, such as at least 90%, such as at least 95% homology or identity thereto, preferably the first strong translational coupler is BCD10 (SEQ ID NO: 16) or a variant thereof having at least 80% homology, such as at least 85%, such as at least 90%, such as at least 95% homology or identity thereto.
38. The method according to any one of items 26 to 37, wherein the second inducible promoter of said second expression module is Pm (SEQ ID NO: 15) or a variant thereof having at least 80% homology, such as at least 85%, such as at least 90%, such as at least 95% homology or identity thereto, and the second strong translational coupler, is BCD10 (SEQ ID NO: 16) or a variant thereof having at least 80% homology, such as at least 85%, such as at least 90%, such as at least 95% homology or identity thereto.
39. The method according to any one of items 26 to 38, wherein the bacterial cell is as defined in any one of items 1 to 25.
40. The method according to any one of items 26 to 39, wherein the concentration of 3-fluorobenzoate or a salt thereof in said medium is at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as at least 25 mM, such as at least 30 mM, such as at least 35 mM, such as at least 40 mM, such as at least 45 mM, such as 50 mM or more.
41. The method according to any one of items 26 to 40, wherein the 2-fluoro-cis,cis-muconate is produced with a titer of at least 500 mg/L, such as at least 750 mg/L, such as at least 1 g/L, such as at least 1.5 g/L, such as at least 2 g/L, such as at least 2.5 g/L, such as at least 3 g/L, such as at least 3.5 g/L, such as at least 4 g/L, such as at least 4.5 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
42. The method according to any one of items 26 to 41, further comprising a step of recovering the 2-fluoro-cis,cis-muconate.
43. The method according to any one of items 26 to 42, further comprising a step of converting the 2-fluoro-cis,cis-muconate to a derivative thereof selected from the group consisting of 2-fluoroadipic acid, [—OC—(F)—(CH₂)₄—CO—NH—(CH₂)₆—NH—]ₙ, 2-fluoro-1,6-hexanediol, fluoro-terephthalic acid, fluoro-trimellitic acid, a fluorinated polyester, a fluorinated polyurethane, a fluorinated acrylic, a fluorinated adhesive and a fluorinated dye.
44. 2-fluoro-cis,cis-muconate or a derivative thereof, preferably wherein the derivative is selected from the group consisting of 2-fluoroadipic acid, [—OC—(F)—(CH₂)₄—CO—NH—(CH₂)₆—NH—]ₙ, 2-fluoro-1,6-hexanediol, a fluorinated polyester, a fluorinated polyurethane, a fluorinated acrylic, a fluorinated adhesive and a fluorinated dye.
45. 2-fluoro-cis,cis-muconate or a derivative thereof obtainable by a method according to any one of items 26 to 43, preferably wherein the derivative is selected from the group consisting of 2-fluoroadipic acid, [—OC—(F)—(CH₂)₄—CO—NH—(CH₂)₆—NH—]ₙ, 2-fluoro-1,6-hexanediol, a fluorinated polyester, a fluorinated polyurethane, a fluorinated acrylic, a fluorinated adhesive and a fluorinated dye.
46. A nucleic acid construct for modifying a bacterial cell comprising:
   a first polynucleotide encoding a native or heterologous benzoate 1,2-dioxygenase (EC: 1.14.12.10) and further comprising a medium-strong constitutive promoter;
   a second polynucleotide encoding a native or heterologous benzoate-1,2-dihydrodiol dehydrogenase (EC: 1.3.1.25); and
   a third polynucleotide encoding a first native or heterologous catechol 1,2-dioxygenase (EC: 1.13.11.1), a first expression module comprising a first promoter, said first expression module further comprising a first strong translation initiation sequence such as a first strong translational coupler; and
   optionally, a fourth polynucleotide encoding a second native or heterologous catechol 1,2-dioxygenase (EC: 1.13.11.1), a second expression module comprising a second promoter, said second expression module further comprising a second strong translational initiation sequence such as a second strong translational coupler.
47. The nucleic acid construct according to item 46, wherein the first and/or the second promoter is a medium-to-weak constitutive promoter.
48. The nucleic acid construct according to item 47, wherein the medium-to-weak constitutive promoter is P_{14b} (SEQ ID NO: 49) or a variant thereof having at least 80% homology, such as at least 85%, such as at least 90%, such as at least 95% homology or identity thereto.
49. The nucleic acid construct according to any one of items 46 to 48, wherein the first and/or the second promoter is a first and/or a second inducible promoter, optionally wherein the first and/or second promoter coupled with a sequence encoding an associated activator protein.
50. The nucleic acid construct according to any one of items 46 to 49, wherein the first and/or the second strong translation initiation sequence has similar translation initiation strength as BCD2 (SEQ ID NO: 22) or BCD10 (SEQ ID NO: 16).
51. A nucleic acid construct for modifying a bacterial cell comprising:
   a first polynucleotide encoding a native or heterologous benzoate 1,2-dioxygenase (EC: 1.14.12.10) and further comprising a medium-strong constitutive promoter;
   a second polynucleotide encoding a native or heterologous benzoate-1,2-dihydrodiol dehydrogenase (EC: 1.3.1.25); and
   a third polynucleotide encoding a first native or heterologous catechol 1,2-dioxygenase (EC: 1.13.11.1), a first expression module comprising a first inducible promoter and a sequence encoding an associated activator protein, said first expression module further comprising a first strong translational coupler.
52. A nucleic acid construct for modifying a bacterial cell comprising:
   a first polynucleotide encoding a native or heterologous benzoate 1,2-dioxygenase (EC: 1.14.12.10) and further comprising a medium-strong constitutive promoter;
   a second polynucleotide encoding a native or heterologous benzoate-1,2-dihydrodiol dehydrogenase (EC: 1.3.1.25);
   a third polynucleotide encoding a first native or heterologous catechol 1,2-dioxygenase (EC: 1.13.11.1), a first expression module comprising a first inducible promoter and a sequence encoding an associated activator protein, said first expression module further comprising a first strong translational coupler; and
   a fourth polynucleotide encoding a second native or heterologous catechol 1,2-dioxygenase (EC: 1.13.11.1), a second expression module comprising a second inducible promoter, said second expression module further comprising a second strong translational coupler.
53. The nucleic acid construct according to any one of items 46 to 52, wherein the benzoate 1,2-dioxygenase is BenABC or a functional variant thereof having at least 80% homology, such as at least 85%, such as at least 90%, such as at least 95% homology or identity thereto, consisting of BenA (SEQ ID NO: 1) or a functional variant thereof having at least 80% homology, such as at least 85%, such as at least 90%, such as at least 95% homology or identity thereto, BenB (SEQ ID NO: 2) or a functional variant thereof having at least 80% homology, such as at least 85%, such as at least 90%, such as at least 95% homology or identity thereto, and BenC (SEQ ID NO: 3) or a functional variant thereof having at least 80% homology, such as at least 85%, such as at least 90%, such as at least 95% homology or identity thereto.
54. The nucleic acid construct according to any one of items 46 to 53, wherein the medium-strong constitutive promoter is P_{tac} (SEQ ID NO: 15), or a variant thereof having at least 80% homology, such as at least 85%, such as at least 90%, such as at least 95% homology or identity thereto.
55. The nucleic acid construct according to any one of items 46 to 54, wherein the benzoate-1,2-dihydrodiol dehydrogenase is BenD (SEQ ID NO: 7) or a functional variant thereof having at least 80% homology, such as at least 85%, such as at least 90%, such as at least 95% homology or identity thereto.
56. The nucleic acid construct according to any one of items 46 to 55, wherein the first catechol 1,2-dioxygenase is CatA-1 (SEQ ID NO: 9) or a functional variant thereof having at least 80% homology, such as at least 85%, such as at least 90%, such as at least 95% homology or identity thereto, and the second catechol 1,2-dioxygenase is CatA-II (SEQ ID NO: 11) or a functional variant thereof having at least 80% homology, such as at least 85%, such as at least 90%, such as at least 95% homology or identity thereto.
57. The nucleic acid construct according to any one of items 46 to 56, wherein the first inducible promoter of said first expression module is the Pm variant ML1-17 (SEQ ID NO: 18) or a variant thereof having at least 80% homology, such as at least 85%, such as at least 90%, such as at least 95% homology or identity thereto, the associated activator protein is XylS (SEQ ID NO: 21) or a functional variant thereof having at least 80% homology, such as at least 85%, such as at least 90%, such as at least 95% homology or identity thereto, and the first strong translational coupler is BCD2 (SEQ ID NO: 22) or BCD10 (SEQ ID NO: 16), or a variant thereof having at least 80% homology, such as at least 85%, such as at least 90%, such as at least 95% homology or identity thereto, preferably the first strong translational coupler is BCD10 (SEQ ID NO: 16) or a variant thereof having at least 80% homology, such as at least 85%, such as at least 90%, such as at least 95% homology or identity thereto.
58. The nucleic acid construct according to any one of items 46 to 57, wherein the second inducible promoter of said second expression module is Pm (SEQ ID NO: 15) or a variant thereof having at least 80% homology, such as at least 85%, such as at least 90%, such as at least 95% homology or identity thereto, and the second strong translational coupler is BCD10 (SEQ ID NO: 16) or a variant thereof having at least 80% homology, such as at least 85%, such as at least 90%, such as at least 95% homology or identity thereto.
59. The nucleic acid construct according to any one of items 46 to 58, wherein the first polynucleotide encoding the benzoate 1,2-dioxygenase comprises or consists of SEQ ID NO: 4 or a variant thereof having at least 80% homology, such as at least 85%, such as at least 90%, such as at least 95% homology or identity thereto, SEQ ID NO: 5 or a variant thereof having at least 80% homology, such as at least 85%, such as at least 90%, such as at least 95% homology or identity thereto, and SEQ ID NO: 6 or a variant thereof having at least 80% homology, such as at least 85%, such as at least 90%, such as at least 95% homology or identity thereto;
   said first polynucleotide further comprising the medium-strong constitutive promoter comprising or consisting of SEQ ID NO: 14 or a variant thereof having at least 80% homology, such as at least 85%, such as at least 90%, such as at least 95% homology or identity thereto.
60. The nucleic acid construct according to any of items 46 to 59, wherein the second polynucleotide encoding the benzoate-1,2-dihydrodiol dehydrogenase comprises or consists of SEQ ID NO: 8 or a variant thereof having at least 80% homology, such as at least 85%, such as at least 90%, such as at least 95% homology or identity thereto.
61. The nucleic acid construct according to any one of items 46 to 60, wherein the third polynucleotide encoding the first catechol 1,2-dioxygenase comprises or consists of SEQ ID NO: 10 or a variant thereof having at least 80% homology, such as at least 85%, such as at least 90%, such as at least 95% homology or identity thereto;
   said third polynucleotide further comprising the first expression module comprising the first inducible promoter comprising or consisting of SEQ ID NO: 18 or a variant thereof having at least 80% homology, such as at least 85%, such as at least 90%, such as at least 95% homology or identity thereto, the associated activator protein comprising or consisting of SEQ ID NO: 20 or a functional variant thereof having at least 80% homology, such as at least 85%, such as at least 90%, such as at least 95% homology or identity thereto, and the first strong translational coupler comprising or consisting of SEQ ID NO: 16 or a variant thereof having at least 80% homology, such as at least 85%, such as at least 90%, such as at least 95% homology or identity thereto.
62. The nucleic acid construct according to any one of items 46 to 61, wherein the fourth polynucleotide encoding the second catechol 1,2-dioxygenase comprises or consists of SEQ ID NO: 12 or a variant thereof having at least 80% homology, such as at least 85%, such as at least 90%, such as at least 95% homology or identity thereto;
   said fourth polynucleotide further comprising the second expression module comprising the second inducible promoter comprising or consisting of SEQ ID NO: 15 or a variant thereof having at least 80% homology, such as at least 85%, such as at least 90%, such as at least 95% homology or identity thereto, and the second strong translational coupler comprising or consisting of SEQ ID NO: 16 or a variant thereof having at least 80% homology, such as at least 85%, such as at least 90%, such as at least 95% homology or identity thereto.
63. The nucleic acid construct according to any one of items 46 to 62, wherein one or more of the polynucleotides is/are codon-optimized for said bacterial cell.
64. A vector comprising or consisting of the nucleic acid construct according to any one of items 46 to 63.
65. The bacterial cell according to any one of items 1 to 25, wherein the bacterial cell comprises a nucleic acid construct according to any one of item 46 to 63.
66. A kit of parts comprising:
   the bacterial cell according to any one of items 1 to 25 and instructions for use; and/or
   the nucleic acid construct according to any one of items 46 to 63 and instructions for use; and optionally the bacterial cell to be modified.

1. A bacterial cell capable of producing 2-fluoro-cis,cis-muconate, in the presence of 3-fluorobenzoate or a salt thereof, said bacterial cell expressing:
benzoate 1,2-dioxygenase (EC: 1.14.12.10), wherein the benzoate 1,2-dioxygenase is capable of converting 3-fluorobenzoate to 3-fluorocyclohexadiene-cis, cis-1,2-diol-1-carboxylate;
a benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
a first catechol 1,2-dioxygenases (EC: 1.13.11.1), wherein the first catechol 1,2-dioxygenases is capable of converting 3-fluorocatechol to 2-fluoro-cis,cis-muconate,
or
a benzoate 1,2-dioxygenase (EC: 1.14.12.10), wherein the benzoate 1,2-dioxygenase is capable of converting 3-fluorobenzoate to 3-fluorocyclohexadiene-cis,cis-1,2-diol-1-carboxylate;
a benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
a first and a second catechol 1,2-dioxygenases (EC: 1.13.11.1), wherein the first and second catechol 1,2-dioxygenases are each capable of converting 3-fluorocatechol to 2-fluoro-cis,cis-muconate,
wherein:
the expression of said benzoate 1,2-dioxygenase is under the control of a medium-strong constitutive promoter, whereby the benzoate 1,2-dioxygenase is constitutively overexpressed;
the expression of said first catechol 1,2-dioxygenase is under the control of a first expression module comprising a first promoter, said first expression module further comprising a first strong translation initiation sequence such as a first strong translational coupler, and
the expression of said second catechol 1,2-dioxygenase is under the control of a second expression module comprising a second promoter, said second expression module further comprising a second strong translational initiation sequence such as a second strong translational coupler,
whereby the bacterial cell is capable of converting 3-fluorobenzoate to 2-fluoro-cis,cis-muconate.

2. The bacterial cell according to claim 1, wherein the first promoter, the second promoter, or the first and the second promoters each is a medium-to-weak constitutive promoter, such as the medium-to-weak constitutive promoter P_{14b} (SEQ ID NO: 49) or a variant thereof having at least 80% homology thereto, whereby the catechol 1,2-dioxygenase is constitutively expressed.

3. The bacterial cell according to claim 1, wherein the first promoter, the second promoter, or the first and the second promoters each is a first and/or a second inducible promoter, respectively.

4. The bacterial cell according to claim 1, said bacterial cell expressing:
a benzoate 1,2-dioxygenase (EC: 1.14.12.10), wherein the benzoate 1,2-dioxygenase is capable of converting 3-fluorobenzoate to 3-fluorocyclohexadiene-cis, cis-1,2-diol-1-carboxylate;
a benzoate-1,2-dihydrodiol dehydrogenase (EC: 1.3.1.25), wherein the benzoate-1,2-dihydrodiol dehydrogenase is capable of converting 3-fluorocyclohexadiene-cis,cis-1,2-diol-1-carboxylate to 3-fluorocatechol; and
a first catechol 1,2-dioxygenase (EC: 1.13.11.1), wherein the catechol 1,2-dioxygenase is capable of converting 3-fluorocatechol to 2-fluoro-cis, cis-muconate,
wherein:
the expression of said benzoate 1,2-dioxygenase is under the control of a medium-strong constitutive promoter, whereby the benzoate 1,2-dioxygenase is constitutively overexpressed; and
the expression of said first catechol 1,2-dioxygenase is under the control of a first expression module comprising a first inducible promoter coupled with a sequence encoding an associated activator protein, said first expression module further comprising a first strong translational coupler, whereby said first catechol 1,2-dioxygenase is expressed in response to 3-fluorobenzoate,
or
the expression of said benzoate 1,2-dioxygenase is under the control of a medium-strong constitutive promoter, whereby the benzoate 1,2-dioxygenase is constitutively overexpressed;
the expression of said first catechol 1,2-dioxygenase is under the control of a first expression module comprising a first inducible promoter coupled with a sequence encoding an associated activator protein, said first expression module further comprising a first strong translational coupler, whereby said first catechol 1,2-dioxygenase is expressed in response to 3-fluorobenzoate; and
the expression of said second catechol 1,2-dioxygenase is under the control of a second expression module comprising a second inducible promoter, said second expression module further comprising a second strong translational coupler whereby said second catechol 1,2-dioxygenase is expressed in response to 3-fluorobenzoate,
whereby the bacterial cell is capable of converting 3-fluorobenzoate to 2-fluoro-cis,cis-muconate.

5. (canceled)

6. The bacterial cell according to claim 1, wherein the first strong translation initiation sequence, the second strong translation initiation sequence, or the first and second strong translation initiation sequence each has similar translation initiation strength as BCD2 (SEQ ID NO: 22) or BCD10 (SEQ ID NO: 16).

7-9. (canceled)

10. The bacterial cell according to claim 1, wherein the bacterial cell is capable of producing 2-fluoro-cis,cis-muconate with a titer of at least 500 mg/L.

11. (canceled)

12. A method of producing 2-fluoro-cis,cis-muconate, in a bacterial cell, said method comprising the steps of providing the bacterial cell and incubating said bacterial cell in a medium in the presence of 3-fluorobenzoate or a salt thereof, said bacterial cell expressing:
a benzoate 1,2-dioxygenase (EC: 1.14.12.10); a benzoate-1,2-dihydrodiol dehydrogenase (EC: 1.3.1.25); and a first catechol 1,2-dioxygenase (EC: 1.13.11.1),
or
a benzoate 1,2-dioxygenase (EC: 1.14.12.10): a benzoate-1,2-dihydrodiol dehydrogenase (EC: 1.3.1.25); and a first and a second catechol 1,2-dioxygenase (EC: 1.13.11.1),
wherein:
the expression of said benzoate 1,2-dioxygenase is under the control of a medium-strong constitutive promoter;
the expression of said first catechol 1,2-dioxygenase is under the control of a first expression module comprising a first promoter, said first expression module further comprising a first strong translation initiation sequence such as a first strong translational coupler; and
the expression of said second catechol 1,2-dioxygenase is under the control of a second expression module comprising a second promoter, said second expression module further comprising a second strong translational initiation sequence such as a second strong translational coupler.

13. The method according to claim 120, wherein said bacterial cell expresses:
a benzoate 1,2-dioxygenase (EC: 1.14.12.10); a benzoate-1,2-dihydrodiol dehydrogenase (EC: 1.3.1.25); and
a first catechol 1,2-dioxygenase (EC: 1.13.11.1),
wherein:
the expression of said benzoate 1,2-dioxygenase is under the control of a medium-strong constitutive promoter;
the expression of said first catechol 1,2-dioxygenase is under the control of a first expression module comprising a first medium-to-weak constitutive promoter, said first expression module further comprising a first strong translational coupler,
or
the expression of said first catechol 1,2-dioxygenase is under the control of a first expression module comprising a first inducible promoter coupled with a sequence encoding an associated activator protein, said first expression module further comprising a first strong translational coupler.

14-16. (canceled)

17. The method according to claim 12, further comprising a step of recovering the 2-fluoro-cis,cis-muconate and further comprising a step of converting the 2-fluoro-cis,cis-muconate to a derivative thereof selected from the group consisting of 2-fluoroadipic acid, [—OC—(F)—(CH₂)₄—CO—NH—(CH₂)₆—NH—]ₙ, 2-fluoro-1,6-hexanediol, fluoro-terephthalic acid, fluoro-trimellitic acid, a fluorinated polyester, a fluorinated polyurethane, a fluorinated acrylic, a fluorinated adhesive and a fluorinated dye.

18. The method of claim 17, wherein said derivative is selected from the group consisting of 2-fluoroadipic acid, [—OC—(F)—(CH₂)₄—CO—NH—(CH₂)₆—NH—]ₙ, 2-fluoro-1,6-hexanediol, a fluorinated polyester, a fluorinated polyurethane, a fluorinated acrylic, a fluorinated adhesive and a fluorinated dye.

19-25. (canceled)

26. The bacterial cell according to claim 3, wherein the first promoter, the second promoter, or the first and the second promoter is each coupled with a sequence encoding an associated activator protein.

27. The bacterial cell according to claim 1, wherein the benzoate 1,2-dioxygenase is BenABC or a functional variant thereof having at least 80% homology thereto, wherein BenABC comprises or consists of BenA (SEQ ID NO: 1) or a functional variant thereof having at least 80% homology thereto, BenB (SEQ ID NO: 2) or a functional variant thereof having at least 80% homology thereto, and BenC (SEQ ID NO: 3) or a functional variant thereof having at least 80% thereto.

28. The bacterial cell according to claim 1, wherein the benzoate-1,2-dihydrodiol dehydrogenase is BenD (SEQ ID NO: 7) or a functional variant thereof having at least 80% homology thereto.

29. The bacterial cell according to claim 1, wherein the first catechol 1,2-dioxygenase is CatA-I (SEQ ID NO: 9) or a functional variant thereof having at least 80% homology thereto.

30. The bacterial cell according to claim 1, wherein the second catechol 1,2-dioxygenase is CatA-II (SEQ ID NO: 11) or a functional variant thereof having at least 80% homology thereto.

31. The bacterial cell according to claim 1, wherein the medium-strong constitutive promoter is P_{tac} (SEQ ID NO: 15), or a variant thereof having at least 80% homology thereto.

32. The bacterial cell according to claim 1, wherein the first promoter of said first expression module is the medium-to-low constitutive promoter P_{14b} (SEQ ID NO: 49) or a variant thereof having at least 80% homology thereto, and the first strong translational coupler is BCD2 (SEQ ID NO: 22) or BCD10 (SEQ ID NO: 16), or a variant thereof having at least 80% homology thereto.

33. The bacterial cell according to claim 1, wherein the second promoter of said second expression module is the medium-to-low constitutive promoter P_{14b} (SEQ ID NO: 49) or a variant thereof having at least 80% homology thereto, and the second strong translational coupler is BCD10 (SEQ ID NO: 16) or a variant thereof having at least 80% homology thereto.

34. The bacterial cell according to claim 4, wherein the first inducible promoter of said first expression module is the Pm variant MIL1-17 (SEQ ID NO: 18) or a variant thereof having at least 80% homology thereto, the associated activator protein is XylS (SEQ ID NO: 21) or a functional variant thereof having at least 80% homology thereto, and the first strong translational coupler is BCD2 (SEQ ID NO: 22) or BCD10 (SEQ ID NO: 16), or a variant thereof having at least 80% homology thereto.

35. The bacterial cell according to claim 3, wherein the second inducible promoter of said second expression module is Pm (SEQ ID NO: 15) or a variant thereof having at least 80% homology thereto, and the second strong translational coupler is BCD10 (SEQ ID NO: 16) or a variant thereof having at least 80% homology thereto.


SEQUENCE LISTING (84 sequences)

SEQ ID NO: 1 - protein, 452 aa, Pseudomonas putida
MSLGFDYLNAMLEDDREKGIYRCKREMFTDPRLFDLEMKHIFEGNWIYLAHESQIPEKNDFLTLTMGRQPIFIARNKDGELNAFLNACSHRGAMLCRHKRGNRSSYTCPFHGWTFNNSGKLLKVKDPSNAGYPDSFNCDGSHDLTKVARFESYRGFLFGSLNADVKPLVEHLGESAKIIDMIVDQSPEGLEVLRGASSYIYEGNWKLTAENGADGYHVSSVHWNYAATQNQRKQREAGDEIKTMSAGAWAKQGGGFYSFDHGHLLLWTRWANPEDRPAYERRDQLAADFGQARADWMIENSRNLCLYPNVYLMDQFSSQIRVARPISVNKTEITIYCIAPKGESADARAKRIRQYEDFFNVSGMATPDDLEEFRSCQTGYGGGTGWNDMSRGAKHWVEGADEAAKEIELEPLLSGVRTEDEGLFVLQHKYWQDTMIQALKDEQQLIPVEAVQ

SEQ ID NO: 2 - protein, 161 aa, Pseudomonas putida
MSLYDTVRDFLYREARYLDDAQWDQWLELYASDASFWMPSWDDDDTLTEDPQSEISLIWYGNRGGLEDRVFRIKTERSSATVPDTRTSHNISNIEIVEQGEDNCQVRFNWHTLSFRYKTTDSYFGTSFYTLDLRGEQPLIKAKKVVLKNDYVRQVIDIYHI

SEQ ID NO: 3 - protein, 336 aa, Pseudomonas putida
MSYQIALNFEDGVTRFIEATGQETVADAAYRQGMNIPLDCRDGACGTCKCKAESGRYDLGDNFIEDALSEDEIAEGYVLTCQMRAESDCVIRIPASSQLCKTEQATFEAAISDVRQLSVSTIALSIKGEALSRLAFLPGQYVNLKVPGSEQSRAYSFSSLQKDGEVSFLIRNVPGGLMSSFLTNLAKAGDSLSLAGPLGSFYLRPIQRPLLLLAGGTGLAPFTAMLEKIAEQGSEHPLHLIYGVTNDFDLVELDRLQALAARIPNFTYSACVANPDSQYPQKGYVTQHIEPRHLNDGDVDVYLCGPPPMVEAVSQYVREQGITPANFYYEKFAAAA

SEQ ID NO: 4 - DNA, 1359 bp, Pseudomonas putida
atgtccctgggattcgactacctcaatgccatgctcgaggacgaccgtgaaaagggcatctaccgctgcaagcgggagatgttcaccgacccacggctgttcgacctggagatgaaacacatcttcgagggcaactggatctacctggcccacgaaagccagatccccgagaagaatgacttcctcaccctgaccatggggcgccagccgattttcatcgcgcgcaacaaggacggtgagctcaatgccttcctcaacgcctgcagccaccgcggtgccatgctgtgccggcacaagcgcggcaaccgttccagctatacctgcccgttccacggctggacgttcaacaacagcggcaaactgctgaaagtgaaagaccccagcaacgccggctaccccgacagcttcaactgcgacggctcccacgacctgaccaaggtggcacgcttcgagtcgtaccggggcttcttgttcggcagcctgaacgccgatgtgaagccgctggtcgagcacctgggcgagtcggcgaagatcatcgacatgatcgtcgatcagtcgcctgaaggcctggaagtgctgcgcggtgccagctcgtacatctacgaaggcaactggaagctcaccgccgaaaacggcgctgacggctaccacgtaagctccgtgcactggaactacgccgccacccagaaccagcgcaagcagcgcgaggcgggtgacgagatcaagaccatgagtgctggcgcctgggccaagcagggcggcggtttctattccttcgaccacggccacctgctgctgtggacccgttgggccaacccggaagaccgcccggcctatgagcgccgcgaccaactggccgccgatttcggccaggcacgtgccgactggatgatcgaaaactcgcgcaacctgtgcctgtaccccaacgtgtacctgatggaccagttcagctcgcagatccgcgttgcccggccgatttcggtgaacaagaccgaaatcaccatctactgcatcgcgccgaaaggcgagagcgccgatgctcgtgccaagcgtattcgccagtacgaagacttcttcaacgtcagcggcatggccaccccggacgacctggaagaattccgctcgtgccagaccggctatggcggcggcactggctggaacgacatgtcccgtggcgcgaaacactgggtcgaaggcgccgatgaggcggccaaggagattgaactcgaacccctgctgtcgggcgtgcgcaccgaggacgaaggcctgttcgtgctgcagcacaagtactggcaggacaccatgatccaggccctcaaggacgaacagcagctgatccccgtggaggccgtgcaatga

SEQ ID NO: 5 - DNA, 486 bp, Pseudomonas putida
atgagcctgtatgacaccgtgcgcgacttcctgtaccgcgaagcgcgctacctggacgatgcccagtgggaccagtggctggaactgtacgccagcgatgccagcttctggatgccgagctgggacgatgacgacaccctcaccgaagacccacaaagcgaaatctcgctgatctggtacggcaaccgtggcggcctggaagaccgtgtgttccgcatcaagaccgagcgctccagtgcgaccgtgcccgacacccgcacctcgcacaacatcagcaacatcgagatcgtcgagcagggcgaggacaactgccaggtgcgcttcaactggcacaccctgagcttccgctacaagaccaccgacagttacttcggcaccagcttctacaccctcgacctgcgcggcgagcagccgctgatcaaggccaagaaggtggtgctgaagaacgactacgtccgccaggtcatcgacatctaccacatctga

SEQ ID NO: 6 - DNA, 1011 bp, Pseudomonas putida
atgagctaccagatcgcactgaatttcgaagacggggttacccgtttcattgaggccaccggtcaggaaaccgtggccgacgccgcttaccgccaaggcatgaacattccgctggactgccgcgacggcgcttgcgggacctgcaagtgcaaggctgagtccggtcgttatgaccttggcgacaacttcatcgaagacgccttgagcgaagacgaaatcgccgaaggttacgtgctgacgtgccagatgcgcgccgaaagcgactgcgtcatccgcatcccggcttcgtcgcagctgtgcaagaccgagcaggccactttcgaggccgccatcagcgatgtgcgccaactgtcggtcagcaccattgccctgtcgatcaagggtgaggccctgagccgtctggccttcctcccggggcagtacgtcaacctgaaggttccgggtagcgagcagagccgggcctactcgttcagctcgctgcagaaggacggcgaagtcagcttcctgatccgcaacgtaccgggtgggctgatgagcagcttcctgaccaaccttgccaaagctggcgacagcctgagcctggccgggccgttgggcagcttctacctgcggccgatccagcgcccgctgttgctgctggccggtggtaccgggctggcaccgttcaccgcgatgctggagaagatcgccgagcagggcagcgagcacccgctgcacctgatctacggcgtgaccaacgactttgatctggtcgaactcgaccgcctgcaagcgctggcggcacgtatccccaacttcacctacagcgcctgcgtcgccaacccggacagccagtacccgcagaagggctatgtcacccagcacatcgagccacgccacctcaatgacggcgatgtggatgtgtacctgtgcggcccgccacccatggtggaagcggtcagccagtacgtgcgtgagcagggcattaccccggccaacttctactacgagaagtttgcggctgcggcctga

SEQ ID NO: 7 - protein, 253 aa, Pseudomonas putida
MNNRFQGKVALVTGAAQGIGRGVCWRLKAEGAQVVAVDRSELVHELAGEGMLTLTADLEQHADCARVMASAVETHGRLDILVNNVGGTIWAKPFEHYEVEQIEAEVRRSLFPTLWCCHAALPYMLERGSGAIVNVSSIATRGVNRVPYGAAKGGVNALTACLAFETAGRGIRVNATAPGGTEAPPRRVPRNSAEQTEQEKRWYQQIVDQTLDSSLMHRYGSIDEQVGAILFLASDEASYITGVTLPVGGGDLG

SEQ ID NO: 8 - DNA, 762 bp, Pseudomonas putida
atgaacaacagattccaaggcaaggttgccctggtcaccggtgccgcccaaggcatcggccggggcgtgtgctggcggctgaaggctgaaggcgcgcaagtggtggcggtagaccgttccgagctggtccacgaactggctggcgagggcatgctcaccctcaccgccgacctggaacaacacgccgattgcgcccgggtcatggccagcgcggtggaaacccatggccgcctggacatcctggtcaacaacgtgggcggcaccatctgggccaagcccttcgagcactacgaagtcgagcagatcgaggccgaggtgcgccgctcgctgttccccaccctgtggtgctgccacgccgcgctgccgtacatgctcgagcgtggcagtggcgccatcgtcaatgtctcgtccatcgctacccgcggcgtcaaccgtgtgccttacggcgcagccaagggtggcgtcaacgcgctcactgcctgcctggccttcgaaaccgccggccggggcatccgcgtcaacgccactgcgcccggcggcaccgaagcaccgccccggcgcgtaccgcgcaacagtgcagagcaaaccgagcaggaaaagcgctggtaccagcagatcgtcgaccagaccctcgacagcagcctgatgcaccgctacggcagcatcgacgagcaggtgggggccattctgttcctcgcgtccgacgaagcctcgtacatcaccggcgtgaccctgccggtagggggcggcgacctgggctga

SEQ ID NO: 9 - protein, 311 aa, Pseudomonas putida
MTVKISHTADIQAFFNRVAGLDHAEGNPRFKQIILRVLQDTARLIEDLEITEDEFWHAVDYLNRLGGRNEAGLLAAGLGIEHFLDLLQDAKDAEAGLGGGTPRTIEGPLYVAGAPLAQGEARMDDGTDPGVVMFLQGQVFDADGKPLAGATVDLWHANTQGTYSYFDSTQSEFNLRRRIITDAEGRYRARSIVPSGYGCDPQGPTQECLDLLGRHGQRPAHVHFFISAPGHRHLTTQINFAGDKYLWDDFAYATRDGLIGELRFVEDAAAARDRGVQGERFAELSFDFRLQGAKSPDAEARSHRPRALQEG

SEQ ID NO: 10 - DNA, 936 bp, Pseudomonas putida
atgaccgtgaaaatttcccacactgccgacattcaagccttcttcaaccgggtagctggcctggaccatgccgaaggaaacccgcgcttcaagcagatcattctgcgcgtgctgcaagacaccgcccgcctgatcgaagacctggagattaccgaggacgagttctggcacgccgtcgactacctcaaccgcctgggcggccgtaacgaggcaggcctgctggctgctggcctgggtatcgagcacttcctcgacctgctgcaggatgccaaggatgccgaagccggccttggcggcggcaccccgcgcaccatcgaaggcccgttgtacgttgccggggcgccgctggcccagggcgaagcgcgcatggacgacggcactgacccaggcgtggtgatgttccttcagggccaggtgttcgatgccgacggcaagccgttggccggtgccaccgtcgacctgtggcacgccaatacccagggcacctattcgtacttcgattcgacccagtccgagttcaacctgcgtcggcgtatcatcaccgatgccgagggccgctaccgcgcgcgctcgatcgtgccgtccgggtatggctgcgacccgcagggcccaacccaggaatgcctggacctgctcggccgccacggccagcgcccggcgcacgtgcacttcttcatctcggcaccggggcaccgccacctgaccacgcagatcaactttgctggcgacaagtacctgtgggacgactttgcctatgccacccgcgacgggctgatcggcgaactgcgttttgtcgaggatgcggcggcggcgcgcgaccgcggtgtgcaaggcgagcgctttgccgagctgtcattcgacttccgcttgcagggtgccaagtcgcctgacgccgaggcgcgaagccatcggccgcgggcgttgcaggagggctga

SEQ ID NO: 11 - protein, 304 aa, Pseudomonas putida
MTVNISHTAEVQQFFEQAAGFCNAAGNPRLKRIVQRLLQDTARLIEDLDISEDEFWHAVDYLNRLGGRGEAGLLVAGLGIEHFLDLLQDAKDQEAGRVGGTPRTIEGPLYVAGAPIAQGEVRMDDGSEEGVATVMFLEGQVLDPHGRPLPGATVDLWHANTRGTYSFFDQSQSAYNLRRRIVTDAQGRYRARSIVPSGYGCDPQGPTQECLDLLGRHGQRPAHVHFFISAPGYRHLTTQINLSGDKYLWDDFAYATRDGLVGEVVFVEGPDGRHAELKFDFQLQQAQGGADEQRSGRPRALQEA

SEQ ID NO: 12 - DNA, 915 bp, Pseudomonas putida
atgaccgtgaacatttcccatactgccgaggtacagcagttcttcgagcaggccgcaggcttttgtaatgcggccggcaacccacgcctcaaacgcatcgtgcagcgcctgctgcaggataccgcgcggctgatcgaagacctggacatcagcgaagacgagttctggcacgccgtcgattacctcaaccgcctgggcggtcgcggcgaagccgggttgctggtggcggggctgggcatcgaacacttcctcgacctgctgcaggatgccaaggaccaggaggcagggcgcgttggcggcaccccacgcaccatcgaaggcccgttgtacgtggctggcgcaccgattgcccaaggtgaagtgcgcatggacgacggcagcgaggagggcgtggccacggtgatgttcctggaaggccaggtgctggacccgcacggacgcccgctgccgggtgccacggtcgacctgtggcatgccaatacccgtggtacctactcgttcttcgaccaaagccagtcggcgtacaacctgcgtcggcgcatcgttaccgatgcccaggggcgctaccgcgcgcgctccatcgtgccatcgggctatggctgcgacccgcaggggccaacccaggaatgcctggacctgctgggccgtcatggccagcgcccggcgcacgtgcacttctttatctcggccccagggtaccggcacctgaccacgcagataaacctgtcgggggacaagtacctgtgggatgactttgcctatgccacacgggatgggctggtcggggaggtggtgttcgtcgaagggccggatggtcggcatgccgagctgaagttcgacttccagttgcagcaggcccagggcggtgccgatgagcagcgcagcgggcggccgcgagctttgcaggaggcctga

SEQ ID NO: 13 - DNA, 48 bp, Pseudomonas putida
tgcacaatccggatagccccccgccagccgtctccctaacctgaccag

SEQ ID NO: 14 - DNA, 29 bp, Artificial
  Promoter
  misc_feature (1)..(29): Ptac
ttgacaattaatcatcggctcgtataatg

SEQ ID NO: 15 - DNA, 69 bp, Pseudomonas putida
tgcaagaagcggatacaggagtgcaaaaaatggctatctctagtaaggcctaccccttaggctttatgc

SEQ ID NO: 16 - DNA, 88 bp, Artificial
  Translational coupling sequence
  misc_feature (1)..(88): BCD10
gggcccaagttcacttaaaaaggagatcaacaatgaaagcaattttcgtactgaaacatcttaatcatgcggaggatcgtttctaatg

SEQ ID NO: 17 - DNA, 88 bp, Artificial
  Translational coupling sequence
  misc_feature (1)..(88): BCD6
gggcccaagttcacttaaaaaggagatcaacaatgaaagcaattttcgtactgaaacatcttaatcatgcgccggaggttttctaatg

SEQ ID NO: 18 - DNA, 68 bp, Artificial
  Promoter
  misc_feature (1)..(68): ML1-17
tgcaagaagcggatacaggagtgcaaaaaatggctatctctagtatagcctacccattaggctttatc

SEQ ID NO: 19 - DNA, 88 bp, Artificial
  Translational coupling sequence
  misc_feature (1)..(88): BCD5
gggcccaagttcacttaaaaaggagatcaacaatgaaagcaattttcgtactgaaacatcttaatcatgcaggggagggtttctaatg

SEQ ID NO: 20 - DNA, 966 bp, Pseudomonas putida
atggatttttgcttattgaacgagaaaagtcagatcttcgtccacgccgagccctatgcagtctccgattatgttaaccagtatgtcggtacgcactctattcgcctgcccaagggcgggcgcccggcaggcaggctgcaccacagaatcttcggatgcctcgatctgtgtcgaatcagctacggcggtagcgtgagggtaatctcgcctggattagagacctgttatcatctgcaaataatactcaaaggccattgcctgtggcgtggccatggccaggagcactattttgcgccgggcgaactattgctgctcaatccggatgaccaagccgacctgacctattcagaagattgcgagaaatttatcgttaaattgccctcagtggtccttgatcgggcgtgcagtgacaacaattggcacaagccgagggagggtatccgtttcgccgcgcgacacaatctccagcaactcgatggctttatcaatctactcgggttagtttgtgacgaagcggaacatacaaagtcgatgcctcgggtccaagagcactatgcggggatcatcgcttccaagctgctcgaaatgctgggcagcaatgtcagccgtgaaattttcagcaaaggtaacccgtctttcgagcgagtcgttcaattcattgaggagaatctcaaacggaatatcagccttgagcggttagcggagctggcgatgatgagtccacgctcgctctacaatttgttcgagaagcacgccggcaccacgccgaagaactacatccgcaaccgcaagctcgaaagcatccgcgcctgcttgaacgatcccagtgccaatgtgcgtagtataactgagatagccctagactacggcttcttacatttgggacgcttcgctgaaaactataggagcgcgttcggcgagttgccttccgacaccctgcgtcaatgcaaaaaggaagtggcttga

SEQ ID NO: 21 - protein, 321 aa, Pseudomonas putida
MDFCLLNEKSQIFVHAEPYAVSDYVNQYVGTHSIRLPKGGRPAGRLHHRIFGCLDLCRISYGGSVRVISPGLETCYHLQIILKGHCLWRGHGQEHYFAPGELLLLNPDDQADLTYSEDCEKFIVKLPSVVLDRACSDNNWHKPREGIRFAARHNLQQLDGFINLLGLVCDEAEHTKSMPRVQEHYAGIIASKLLEMLGSNVSREIFSKGNPSFERVVQFIEENLKRNISLERLAELAMMSPRSLYNLFEKHAGTTPKNYIRNRKLESIRACLNDPSANVRSITEIALDYGFLHLGRFAENYRSAFGELPSDTLRQCKKEVA

SEQ ID NO: 22 - DNA, 88 bp, Artificial
  Translational coupling sequence
  misc_feature (1)..(88): BCD2
gggcccaagttcacttaaaaaggagatcaacaatgaaagcaattttcgtactgaaacatcttaatcatgctaaggaggttttctaatg

SEQ ID NO: 23 - DNA, 88 bp, Artificial
  Translational coupling sequence
  misc_feature (1)..(88): BCD1
gggcccaagttcacttaaaaaggagatcaacaatgaaagcaattttcgtactgaaacatcttaatcatgcacaggagactttctaatg

SEQ ID NO: 24 - DNA, 30 bp, Pseudomonas putida
gtctaaacaataacaagggagaccctggcc

SEQ ID NO: 25 - DNA, 134 bp, Pseudomonas putida
cgctccctgattcaaagcaatacgccgaccctgtgggagcgggcgagcccgctcccacagtgatcgcgcagcctttcaggctttgagcaagagagttgctgccacagggtctctcgcgaactggagatttacat

SEQ ID NO: 26 - protein, 146 aa, Vitreoscilla stercoraria
MLDQQTINIIKATVPVLKEHGVTITTTFYKNLFAKHPEVRPLFDMGRQESLEQPKALAMTVLAAAQNIENLPAILPAVKKIAVKHCQAGVAAAHYPIVGQELLGAIKEVLGDAATDDILDAWGKAYGVIADVFIQVEADLYAQAVE

SEQ ID NO: 27 - DNA, 441 bp, Artificial
  Bacterial haemoglobin
  misc_feature (1)..(441): Codon-optimized VHb
atgttagaccagcaaaccattaacatcatcaaagccactgttcctgtattgaaggagcatggcgttaccattaccacgactttttataaaaacttgtttgccaaacaccctgaagtacgtcctttgtttgatatgggtcgccaagaatctttggagcagcctaaggctttggcgatgacggtattggcggcagcgcaaaacattgaaaatttgccagctattttgcctgcggtcaaaaaaattgcagtcaaacattgtcaagcaggcgtggcagcagcgcattatccgattgtcggtcaagaattgttgggtgcgattaaagaagtattgggcgatgccgcaaccgatgacattttggacgcgtggggcaaggcttatggcgtgattgcagatgtgtttattcaagtggaagcagatttgtacgctcaagcggttgaataa

SEQ ID NO: 28 - protein, 246 aa, Pseudomonas putida
MPEHAPDNSRRDFLRKTLTLIPVVTVASTGLGAGASQLLAAPQQQPNVPATPPAGDYRPTFFSAEEWAFVQAAVSRIIPADELGPGALEAGAAEFIDRQMNTPYATGAQWYMQGPFNADAPPELGYQLQLSPQQIYRLGIAAVDGWCKANGGQVFAAQDSATRDRILSKLEAGELVFESVPAKVFFSLLVQNTREGFFCDPIHGGNKGMVGWTQIGFPGARADFMDWVERNEPYPFPAVSIRGERA

SEQ ID NO: 29 - DNA, 741 bp, Pseudomonas putida
atgcctgagcatgccccagacaactcgcgcagggattttctgcgcaagaccttgaccctgatccctgtcgttaccgtggccagcacgggccttggcgcaggcgccagccagctgcttgcggccccgcaacagcaaccgaacgtgcccgccacgccacctgcgggcgactatcgaccgacattcttcagcgccgaggagtgggcttttgtccaggctgccgtgtcgcgcatcatcccggccgatgagcttggcccaggggcactcgaagcgggtgccgccgagttcatcgaccgccagatgaataccccctacgcgactggcgcacagtggtacatgcaagggccgttcaacgccgacgccccgcccgaactgggctaccagctgcagctcagcccgcaacaaatctaccgcctgggcatcgctgccgtggatggctggtgcaaggccaatggtggacaggtttttgctgcgcaagatagcgctacccgggaccgcattctcagcaaattggaggcaggagagctggttttcgaatccgttccggccaaggtgttcttcagcctgctggtgcaaaacacgcgtgaagggttcttctgcgacccgattcatggcggcaacaaaggaatggtcggctggactcagatcggcttccccggcgcccgcgccgatttcatggactgggtcgagcgcaatgagccttacccgttcccggctgtatcgatccgtggtgagagggcctga

SEQ ID NO: 30 - protein, 594 aa, Pseudomonas putida
MATVLNKVDAVIVGFGWAGAIMAKELTEAGLHVVALERGPMQDTYPEGSYPQVIDELTYSVRKKLFVDVSKETVTVRHSVNDVALPNRQLGAFLPGKGVGGAGLHWSGVHFRVDPVELRLRSHYEERYGRTFIPEGMTIQDFGVSYEELEPYFDFAEKVFGTSGQAWTVKGQVVGEGKGGNPYAPDRSNPFPLPAQKNVVSARLFEKAATSLGYKPYNLPSANTSGPYTNPYGAQMGPCNFCGFCSGYVCYMYSKASPNVNILPALRQVPNFELRPNAHVLRVNLDDSKRRATGVTYIDAQGREVEQPAELVILAAFQFNNVRLMLLSGIGKPYDPVKNEGVVGRNFAYQNMGTVKAFFDKDTYTNNFIGAGGNGIAIDDFNADNFDHGPHGFVGGSPMWVNQAGSRPIAGTSNPPGTPAWGSAWKKATADYYTHQVSMDAHGAHQSYRGNYLDLDPTYRDAYGQPLLRMTFDWQENDIKMNQFMVDKLSKIAQAMNPKAIAVLGKQVKDHFNTTSYQTTHLNGGAIMGTDPKTSALNRYLQSWDVHNVFVPGASAFPQGLGYNPTGLVAALTYWSARAIREQYLKNPGPLVQA

SEQ ID NO: 31 - DNA, 1785 bp, Pseudomonas putida
atggcgacggtgttgaacaaagtggatgcggtgatcgtcggcttcggctgggccggtgcgatcatggccaaagagctgaccgaggccgggctgcacgtggtagccctggagcgggggccgatgcaggatacctaccccgaaggcagctacccgcaggtgatcgatgagctgacctacagcgtgcgcaagaagctgttcgtcgatgtatcgaaagaaaccgtcactgtccgccatagcgtgaatgacgtggcattgcccaatcgccagctgggcgcattcctgccgggcaagggcgtggggggcgcgggcctgcactggtcgggtgtgcacttccgggtcgaccccgtcgagttgcgcctgcgcagccactatgaagagcgctacgggcgcacgttcattcccgaaggcatgactatccaggacttcggcgtcagttacgaagagctggagccctatttcgactttgccgaaaaggtctttggcacttcaggccaggcctggaccgtcaaaggccaggtggtcggtgaagggaagggcggcaacccctatgcgccggaccgttccaacccgttcccgctgcccgcgcagaagaacgttgtatcggcaaggctgttcgaaaaggctgccaccagcctgggctacaaaccctacaacctgccgtcggccaacacgtcggggccatacaccaacccctacggggcgcagatggggccttgcaacttctgcggtttctgcagcggctacgtgtgttacatgtactccaaggcctcgcccaacgtgaatatcctgccggcgctgcgccaggtgcccaacttcgagttgcggcccaatgcccatgtgctgcgggtgaacctggacgacagcaagcgcagggccaccggcgtgacctacatcgatgcccaggggcgcgaggtggagcagccggcagagctggtgattctggcggcgttccagttcaacaacgtgcgtctgatgctgctttccggcattggcaaaccttacgacccggtaaagaacgagggtgtggtcgggcgtaacttcgcctatcagaacatgggtacagtgaaggcgttcttcgacaaggacacctacaccaacaacttcatcggcgcgggtggcaatggcattgccatcgacgatttcaacgccgacaacttcgaccatggcccgcacggcttcgtcggcggttcgccaatgtgggtgaaccaggccggcagccggccgattgccggtacctccaacccgccgggcactccggcctggggcagtgcctggaagaaagccacggccgactactacacccaccaggtgtcgatggacgcccacggcgcacaccagtcctaccgtggcaattacctggacctggaccccacctaccgcgatgcctacggtcagccgctgctgcgcatgacgttcgactggcaggaaaacgacatcaagatgaaccagttcatggtcgacaagctgagcaagatcgcccaggcgatgaaccccaaggccattgccgtgctgggcaagcaggtcaaggaccacttcaacaccaccagttaccagaccacccacctgaacggtggggcgatcatgggcactgaccccaagaccagtgcactgaaccgctacctgcaaagctgggacgtacacaacgtgttcgttccgggggcttcggcgttcccgcaggggctgggttacaacccgaccgggctggtggctgcgctcacgtactggtcggcccgcgccatccgtgagcagtacctgaaaaaccccggtccgttggtccaggcttga

SEQ ID NO: 32 - protein, 417 aa, Pseudomonas putida
MSMKTLLIATLVLGAGAAAQAVANDDAQVRLGEYLARAGDCVACHTAKGGKPFAGGLPMETPIGTVYSTNITPAASGIGQYSFEDFDQAVRRGIGKDGSTLYPAMPYPSYARVSEQDMQALYAYFMKGVAPVEQANKASDIPWPLSMRWPLAIWRGVFAPEAKPWQASATADPVVNRGAYLVEGLGHCGACHTPRALTMQEKALSAADGEQFLAGSAPLEGWIAKNLRGDHKDGLGSWSEAQLVQFLKTGRSDRSAVFGGMSDVVEHSMQHMSDADLTAIARYLKTLPPSNPDDQLHVYDKQVADALWKGDDSKPGAAVYIDNCAACHRTDGQGYTRVFPALAGNPVVQTADATSLIHVVLAGGTVPATHSAPSNFTMPAFGWRLSDQEVAEVVNFIRSSWGNQGSAVTAGDVKSLR

SEQ ID NO: 33 - DNA, 1254 bp, Pseudomonas putida
atgagcatgaagacactgttgatcgccaccctggtgctgggcgccggtgcggctgcacaggccgtagcgaatgacgatgcgcaggtacgcctgggcgagtacctggcccgcgccggtgactgcgtggcctgccataccgccaaaggcggcaaaccctttgccggcgggttgccgatggaaacgccgattggcacggtgtactccaccaacatcaccccggcggccagtggcattggccagtacagcttcgaagacttcgaccaggccgtacgcagggggatcggcaaggacggcagtacgctctatccggccatgccatacccgtcgtatgcgcgggtcagcgagcaggacatgcaggcgctctacgcctatttcatgaagggcgtggcgcctgtcgagcaggcgaacaaggcctctgacattccctggccgctgagcatgcgctggccactggcaatctggcgcggcgtgttcgcaccagaggccaagccctggcaggcatcggcgacagctgaccctgtggtgaaccgcggtgcgtacctggtcgagggccttgggcattgtggcgcttgtcatacgccgcgggcgctgaccatgcaggagaaggctctgagtgcggccgatggcgagcagtttctggccggcagcgcgccgctggaaggctggattgccaagaacctgcgtggcgatcacaaagacggcctgggcagctggagcgaagcgcagttggtgcagtttctcaagactgggcgcagtgatcgcagcgccgtgttcggcggcatgagcgatgtggtcgagcacagcatgcagcacatgagtgatgccgacctgaccgctatcgcccgctacctgaaaaccctgccgccgagtaatccggatgaccagctacatgtgtacgacaagcaggtagccgatgcgctatggaagggtgacgacagcaagcctggggcggcggtgtacatcgacaactgcgcggcctgccaccgcaccgacgggcagggctacacccgcgtgttcccggctttggccggcaacccggtggtgcagactgcggatgccacgtcgctgatccatgtggtgctcgccggtggcacggtaccggcaacgcacagtgcaccgtcgaacttcaccatgccggctttcggctggcgcctgagtgaccaggaggttgccgaggtggtgaatttcattcgcagcagttggggtaaccaaggcagtgccgtcaccgccggtgatgtaaagtcgcttcgctga

SEQ ID NO: 34 - protein, 803 aa, Pseudomonas putida
MSTEGANQGSRWLPRLIGALLLLMGLALLAGGIKLSQLGGSLYYLIAGIGFALSGVLLLAQRQIALGLYGLVLLGSTVWALFEVGLDWWQLVPRLAIWFAIGVVLLLPWARRPLIGPASKANTALLGVAVVASGACALASQFTHPGEVFGELGRDSSEMASAAPAMPDGEWQAYGRTEHGDRYSPLRQITPQNAYRLEEAWRIRTGDLPTENDPVELTNQNTPLKVNGMLYACTAHSRLLALDPDTGAEIWRYDPQVKSPTGTFKGFAHMTCRGVSYYDENRYVSRDGSPAPKITDAGQAVAQACPRRLYLPTADARLIAINADNGKVCEGFANQGVIDLTTGIGPFTAGGYYSTSPAAITRDLVIIGGHVTDNESTNEPSGVIRAYDVHDGHLVWNWDSNNPDDTKPLAAGKMYSRNSANMWSIASVDEDLGMIYLPLGNQTPDQWGADRTPGAEKYSAGVVALDLATGKARWNYQFTHHDLWDMDVGSQPTLVHLKTDDGVKPAIIVPTKQGSLYVLDRRDGTPIVPIREIPTPQGAVEGDHTSPTQARSDLNLLGPELTEQAMWGATPFDQMLCRIQFRELRYEGQYTPPSEQGSLVYPGNVGVFNWGSVSVDPVRQLLFTSPNYMAFVSKMVPREQVAEGSKRESETSGVQPNTGAPYAVIMHPFMSPLGVPCQAPAWGYVAAIDLFTNKVVWKHKNGTTRDSTPLPIGLPVGVPSMGGSIVTAGGVGFLSGTLDQYLRAYDVNNGKELWKARLPAGGQATPMSYTGKDGKQYVLVTAGGHGSLGTKMGDYIIAYKLAE

SEQ ID NO: 35 - DNA, 2412 bp, Pseudomonas putida
atgagcactgaaggtgcgaaccaaggaagccgctggctaccgcgcctgattggcgcgctgctgttgctgatgggcctggccctgctggccggcggtatcaagctgagccagctgggcggatcgctgtactacctgatcgccggtattggctttgccctttcgggcgtcctgctgctggcccaacgccagatcgccctgggcctgtatggcctggtgctgctgggcagcaccgtgtgggccctgttcgaagtaggcctggactggtggcaactggtgccacggctggctatctggttcgccatcggcgtggtgttgctgctgccgtgggcacgtcgcccgctgatcggcccagccagcaaagccaacactgcactgctcggcgtggcagtggtcgcttcgggcgcttgcgcgctggccagccagttcacccatcccggtgaagtgttcggcgaactgggccgcgacagcagcgaaatggccagcgccgccccggccatgcccgacggcgaatggcaggcctacggccgtaccgagcatggcgaccgctactcgccgctgcgccagatcaccccgcagaacgcctaccgtctggaagaagcctggcgcattcgcaccggtgatctgccaaccgaaaacgacccggtggagctgaccaaccagaacaccccgctgaaggtcaacggcatgctttacgcctgcaccgcgcacagtcgcctgctggcgctggacccggacactggcgcagaaatctggcgctacgacccgcaggtcaagagcccgaccggcaccttcaagggctttgcccacatgacctgccgtggcgtctcgtactatgacgaaaaccgctacgtcagccgcgacggcagcccggcgccgaaaattaccgatgcaggccaggccgtggcccaagcctgcccgcgtcgcctctatctgcctaccgcggatgcccgcctgatcgccatcaacgccgacaacggcaaggtctgcgaaggcttcgccaaccagggcgtgatcgacctcaccaccggcattggcccattcaccgccggcggctattactccacctcgcctgctgcgattacccgtgacctggtgatcatcggtggccacgtcaccgacaacgagtcgaccaatgagccgtccggggtgatccgcgcctacgacgtgcacgacggccacctggtgtggaactgggacagtaacaacccggacgacaccaagccattggctgccggcaaaatgtacagccgcaactcggccaacatgtggtcgatcgccagcgtcgacgaagaccttggcatgatctacctgccgctgggcaaccagaccccggaccagtggggcgccgaccgcaccccgggcgccgagaagtacagcgccggcgtggtcgcccttgacctggccaccggcaaggcacgctggaactatcagttcacccaccacgacctgtgggacatggacgtcggcagccagccgaccctggtacacctgaaaaccgacgatggtgtgaaaccggcgatcatcgtaccgaccaagcaaggcagcctgtacgtgctcgaccgccgcgacggtacgccaatcgtgccgatccgcgagatccccaccccacaaggcgcagtggaaggcgaccacacctcgcccacccaggcccgctccgacctcaacctgctcggcccagagctgaccgaacaggccatgtggggcgccacgcctttcgaccagatgctgtgccgcatccagttccgcgaactgcgctacgaaggccagtacaccccgccatccgaacaaggttcgttggtctaccccggcaacgtcggtgtattcaactggggcagcgtgtcggtcgacccggtgcgccagctgctgttcacttcgcccaactacatggcgttcgtgtcgaagatggtcccgcgtgagcaggttgccgaaggcagcaagcgcgaaagcgagaccagcggcgtgcagccgaacaccggcgcaccgtatgcagtgatcatgcacccgttcatgtcgccgctcggtgtaccgtgccaggcacccgcctggggctacgtcgccgccatcgacctgttcaccaacaaggtggtgtggaaacacaagaacggcaccacccgcgacagcaccccgctaccgatcggcctgccggttggcgtgccgagcatgggtggctcgatcgtcaccgccggtggcgtcggcttcctcagcggcacgctcgaccagtacctgcgcgcctatgacgtgaacaacggcaaggagttgtggaaagcacgcctgccagcgggtggccaggctacgccgatgagctacaccggcaaggacggcaagcagtacgtgctggtgactgccggcggccatggctcgctgggcaccaagatgggcgattacatcattgcttacaaattagccgagtaa

SEQ ID NO: 36 - protein, 259 aa, Pseudomonas putida
MRIISVNVNGIQAAAERGLLSWLQAQNADVICLQDTRASAFELDDPAFQLDGYFLYACDAEVPAQGGVALYSRMQPKAVITGLGFETADRYGRYLQADFDKVSIASLLLPSGMNGDEDLNQKFKLMDDFAKYLDKQRRKRREYIYCGSFYVAQQKLDIKNWRDSQQSPGFLAPERAWMDAITGEMGYVDALREVSREGDQYSWWPDNEQAEMLNLGYRFDYQILTPGLRRFVRNARLPRQPRFSQHAPLIVDYDWTLTI

SEQ ID NO: 37 - DNA, 780 bp, Pseudomonas putida
atgcggatcatcagtgtgaacgtaaatggcattcaggctgcggccgagcgtggtttgctcagctggctgcaagcccagaatgccgacgtcatctgccttcaggatacccgcgcctcggcctttgaactcgacgacccagctttccagctcgatggctatttcctttatgcctgcgacgcggaggtgcctgcccaaggtggtgtggccctttactcgcgcatgcagcccaaggcagtcatcaccggcctgggcttcgagacagccgaccgctacgggcgttacctgcaagcagatttcgacaaagtcagtattgccagcctgctgctgccttcgggcatgaacggcgacgaagacttgaatcagaaattcaagttgatggacgacttcgccaagtacctggacaagcagcgtcgcaagcgtcgcgaatacatctattgcggctcgttctacgtggcgcagcagaagctcgacatcaagaattggcgtgacagccagcagtcgccgggcttcctggcgccagaacgcgcctggatggatgcgatcactggcgagatggggtatgtcgatgccttgcgcgaagtcagccgtgaaggcgaccagtacagctggtggccggacaacgagcaggccgagatgctcaacctggggtatcggttcgactaccagatcctcaccccgggcctgcgccgcttcgtgcgcaacgctcgcctgccgcgtcagccacgcttctcccagcatgcaccgctgatcgtcgactatgactggacgctgaccatctaa

SEQ ID NO: 38 - protein, 442 aa, Pseudomonas putida
MRTLDVHPIIDNARFTPFHWMVMAWCGLLLIFDGYDLFIYGVVLPVIMKEWGLTPLQAGALGSYALFGMMFGALAFGSLADRIGRKKGIAICFALFSGATILNGFASNPSEFGIYRFIAGLGCGGLMPNAVALMNEYAPKRLRSTLVAIMFSGYSLGGMLSAGVGIFMLPRFGWESMFFAAAVPLLLLPVILYYLPESIGFLVRQGRIEEARTLLKRLDPDCDVQADDVLQAADRKGSGASVLELFRNGLAIRTLALWVAFFCCLLMVYALSSWLPKLMANAGYSLGSSLSFLLALNFGGMAGAILGGWLGDRYNLVKVKVAFFIAAALSISLLGVNSPMPVLYLLIFIAGATTIGTQILLYAGAAQMYGLSVRSTGLGWASGIGRNGAIVGPLLGGALMGINLPLQLNFIAFAIPGAIAALAMAVHLASGRRHAQTLAAQA

SEQ ID NO: 39 - DNA, 1329 bp, Pseudomonas putida
atgcgaacccttgatgtacacccgatcatcgacaatgcgcgctttaccccgtttcactggatggtcatggcctggtgcggcctgctgctgattttcgacggctatgacctgttcatctacggtgtggtactgccggtcatcatgaaagagtggggcctgaccccgttgcaggctggtgcactgggcagctatgcgctgttcggcatgatgttcggcgccctggccttcggcagcctggccgaccgcatcgggcgcaagaagggcattgccatctgtttcgccttgttctcgggggcgaccatcctcaatggctttgccagcaacccgagcgagtttggcatctaccgcttcatcgccggcctgggctgtggcggcctgatgcccaacgccgtggcactgatgaacgaatacgcacccaagcgcctgcgcagcacgctggtggcgatcatgttcagtggttattcgctgggcggcatgctgtcggcaggtgtcggcatcttcatgctgccgcgttttggctgggagtcgatgttcttcgccgcagcggtgccactgctgctgttaccggtgattctctactacctgcctgaatccatcggcttcctggtacgccaaggccgtatcgaggaagcacgcacattgctcaaacggctggacccggactgcgatgtccaggccgatgatgtgcttcaggcggccgaccgcaaaggcagcggcgcttcggtgctggaactgttccgcaatggcctggcgattcgcaccctggcgctgtgggttgcgttcttctgctgcctgctgatggtctacgccctgagctcctggctgccaaagctgatggccaacgccggctacagcctgggctcaagcctgtcattcctgctggcactgaactttggcggcatggccggggcgatcctcggcggctggctgggtgaccgctacaacctggtcaaggtgaaggtggcgttcttcattgccgccgcactgtcgatcagcctgctgggggtaaacagcccgatgccggtgctgtacctgctgatcttcattgccggcgccaccaccattggtacacagattctgctgtacgccggcgctgcacagatgtacggcctgtcggtgcgctctaccggcctgggctgggcctcgggcatcggccgtaatggcgccatcgtcggcccgctgctgggcggtgcgctgatgggcatcaacctgccactgcaactcaatttcatcgcctttgccatccctggcgccatcgctgcgctggccatggccgtgcacctggccagtggtcggcgccacgcccagaccctggcggcgcaggcctga

SEQ ID NO: 40 - protein, 423 aa, Pseudomonas putida
MPAKQPTPKQEPATKAGRCLARSFMKTLIKDCSVSAIVAGSIATTISYAGPLVIIFHAAEAAGLSHQQLSSWVWAVSMGSALLGALLSLRYRVPVVIAWSIPGSALLVTALPQLGLEQAVGAYLVANLVLLLIGISGAFDRIIARLPGSIAAGMQAGILFSFGIEVFRALPVQPLLVLAMFVTYVLMRRVQPRYAVAAVLGVGMLVTVASGTLRSEALVLELASPQWITPQFSLSAVFSLALPMVLVALTGQFMPGMAVLRNAGYPTPASPLISASALIGVLLAPFGCHGLNLAAVTASLCTGHEAHENPQRRYVAAVAGSVLYLLLGIAGATLMSLFAAFPAVLIAALAGLALYGAISEALVRSLAEPAERDAGLFTFLVTASGVAFLGLSAAFWGLLFGLLAHALLRLRQPRAREVLRRTP

SEQ ID NO: 41 - DNA, 1272 bp, Pseudomonas putida
atgcccgcgaagcagccaacacccaaacaagagcccgccacaaaggccggccgctgccttgcgagaagcttcatgaaaacgctgatcaaggactgctctgtgtcggccatcgtcgccggcagcattgccacgacgatctcctacgccggcccgttggtgatcattttccacgccgccgaagcggccggcctgtcgcaccagcagttgtcatcctgggtctgggccgtgtccatgggcagtgcgttgctcggtgcgctgctcagcctgcgctaccgcgtgccggtagtaatcgcctggtccatcccagggtcggccctgttggtcaccgccttgccgcaactgggcctggagcaggccgtgggcgcctacctggtggccaacctggtcctgctgctgatcggcatcagcggggccttcgaccgcatcatcgcgcgactgccggggtctatcgctgccggtatgcaggccgggatcctgttcagctttggcatcgaggtgttccgcgccttgccggtgcaaccgctgctggtactggcgatgttcgtcacctacgtgctgatgcgtcgggtgcagccgcgctatgcagtggctgcggtactgggcgtgggcatgctggtgaccgtggccagcggtacgttgcgcagcgaggcactggtgctggaactggcctcaccccagtggatcaccccccagttcagcctgtcggcggtattcagcctggcgctgccgatggtgctggtggcgctgaccgggcaattcatgccgggtatggcggtactgcgcaacgctggctaccccacgccggccagcccgctgatcagcgccagtgccctgatcggagtgctactggcgccgttcggttgccacgggctgaacctggcggcggttaccgccagcctgtgcaccggccatgaagcccatgaaaacccgcaacggcgctatgtagcggcggtggctggcagtgtgttgtacctgctactgggcattgccggggccaccttgatgtcgctgttcgccgcctttcccgccgtactgatcgctgccctggccggcttggccctgtatggcgccatcagcgaagccctggtccgcagcctggcagaaccggccgagcgcgacgccgggctgttcaccttcctggtcaccgcttcgggggtggctttcctgggcctgtcggcagcgttctgggggttgctgttcggcctgctggcgcacgcgctgctgaggctgcgccagccacgtgcccgggaggtgctgcgccgtaccccatga

SEQ ID NO: 42 - protein, 416 aa, Pseudomonas putida
MNHTPCVALGLTLLSQPLLAAEGGFFEDSKATLSARNYYFSRDFSDIVGANKQSKAEEWAQGFILDFKSGYTPGPVGFGVDALGLLGIKLDSSPDRTNTGLLPVHGDGRAADEYSRLAPTFKARLSKTELRAGELQPNLPVLTFSDIRLLPPTYQGVSLSSSEIDGLTMQAGHLKSTHLRNEGGDGKMNAMLGHVPMRQAESDAFNYVGGDYAFNANQSSVSLWYGQLEDIYQQGFVGLKHSKPVGDWVLSANLGYFTAREQGDALLGNIDNQAFFSLFTARHGGHSFHAGYQGIYGDSPFPRVFANISPLGNEVPTYEFAYTDERSYQVRYDYNFAAMGVPGLTATVRYITGNNVDTGLGYEGRDRERDLDIGYAVQSGALKGLGIRVRNAMARSNYRSDIDENRLTLVYTWQLF

SEQ ID NO: 43 - DNA, 1251 bp, Pseudomonas putida
atgaaccacaccccttgcgttgccctgggcctgacactgctcagccagccactgctggctgccgaaggcggcttcttcgaggactcgaaggccaccctgagtgcccgtaactactacttcagccgcgatttttccgacattgtcggggccaacaaacagtccaaggccgaggagtgggcccagggtttcatcctcgacttcaaaagtggctacaccccgggcccggtcggttttggcgtggacgctctgggcttgctgggcatcaagctggacagcagtccagaccgcaccaacaccggcctgttgccggtgcatggcgacggccgcgctgccgacgagtacagccgcctggcgccgaccttcaaggcgcggttatcaaaaaccgaactgcgtgcaggtgaactgcaaccgaacctgccggtgctgaccttcagtgatattcgcctgttgccacccacctatcagggcgtgagtttgagctcgtcggaaatcgacgggctgactatgcaggccgggcacttgaaaagcacccacctgcgtaacgaaggtggcgatggcaagatgaacgcgatgctcggccatgtgccgatgcgacaggccgaaagcgacgcgttcaactatgtgggtggggactatgcgttcaatgccaaccaaagcagcgtcagcctttggtacggtcagctggaggacatctaccaacaaggctttgttggcctgaagcacagcaaacccgtcggcgactgggtactcagtgccaacctgggttatttcaccgcgcgtgagcaaggcgatgcgctgctgggcaacatcgacaaccaggcgttcttctcgctgttcactgcccggcatggcgggcacagcttccatgccggctaccagggcatttatggcgatagcccgttcccacgggtatttgccaacatctcgccgttgggcaacgaggtgccgacctatgagtttgcctacaccgacgaacgctcgtaccaggtacgttacgactacaacttcgccgccatgggcgtgccgggcctgacagcgaccgtgcgctacataaccggtaacaatgtcgataccgggctgggttacgaggggcgtgatcgagagcgtgatctggatatcggctatgcggtgcagagcggggcactgaaagggttggggatacgggtgaggaacgccatggcgcgctccaactaccgcagtgatatcgacgagaaccgcctgacgctggtgtacacctggcagctgttctga

SEQ ID NO: 44 - DNA, 88 bp, Artificial
  Translational coupling sequence
  misc_feature (1)..(88): BCD11
gggcccaagttcacttaaaaaggagatcaacaatgaaagcaattttcgtactgaaacatcttaatcatgcgggggagtgtttctaatg

SEQ ID NO: 45 - DNA, 88 bp, Artificial
  Translational coupling sequence
  misc_feature (1)..(88): BCD7
gggcccaagttcacttaaaaaggagatcaacaatgaaagcaattttcgtactgaaacatcttaatcatgctggggagggtttctaatg

SEQ ID NO: 46 - DNA, 35 bp, Artificial
  Promoter
  misc_feature (1)..(35): J23108 promoter
  misc_feature (1)..(35): J23108
ctgacagctagctcagtcctaggtataatgctagc

SEQ ID NO: 47 - DNA, 35 bp, Artificial
  Promoter
  misc_feature (1)..(35): J23114
tttatggctagctcagtcctaggtacaatgctagc

SEQ ID NO: 48 - DNA, 35 bp, Artificial
  Promoter
  misc_feature (1)..(35): J23115
tttatagctagctcagcccttggtacaatgctagc

SEQ ID NO: 49 - DNA, 41 bp, Artificial
  Promoter
  misc_feature (1)..(41): P14b
tttatttgacatgcgtgatgtttagaattataatttgggga

SEQ ID NO: 50 - DNA, 41 bp, Artificial
  Promoter
  misc_feature (1)..(41): P14g
gcccattgacaaggctctcgcggccaggtataattgcacga

SEQ ID NO: 51 - DNA, 966 bp, Artificial
  Nucleotide 134(G) in xylS replaced by C
  misc_feature (1)..(966): xylS.2
atggatttttgcttattgaacgagaaaagtcagatcttcgtccacgccgagccctatgcagtctccgattatgttaaccagtatgtcggtacgcactctattcgcctgcccaagggcgggcgcccggcaggcacgctgcaccacagaatcttcggatgcctcgatctgtgtcgaatcagctacggcggtagcgtgagggtaatctcgcctggattagagacctgttatcatctgcaaataatactcaaaggccattgcctgtggcgtggccatggccaggagcactattttgcgccgggcgaactattgctgctcaatccggatgaccaagccgacctgacctattcagaagattgcgagaaatttatcgttaaattgccctcagtggtccttgatcgggcgtgcagtgacaacaattggcacaagccgagggagggtatccgtttcgccgcgcgacacaatctccagcaactcgatggctttatcaatctactcgggttagtttgtgacgaagcggaacatacaaagtcgatgcctcgggtccaagagcactatgcggggatcatcgcttccaagctgctcgaaatgctgggcagcaatgtcagccgtgaaattttcagcaaaggtaacccgtctttcgagcgagtcgttcaattcattgaggagaatctcaaacggaatatcagccttgagcggttagcggagctggcgatgatgagtccacgctcgctctacaatttgttcgagaagcacgccggcaccacgccgaagaactacatccgcaaccgcaagctcgaaagcatccgcgcctgcttgaacgatcccagtgccaatgtgcgtagtataactgagatagccctagactacggcttcttacatttgggacgcttcgctgaaaactataggagcgcgttcggcgagttgccttccgacaccctgcgtcaatgcaaaaaggaagtggcttga

SEQ ID NO: 52 - protein, 321 aa, Artificial
  Amino acid 45(Arg) in XylS replaced by Thr
  MISC_FEATURE (1)..(321): XylS.2
MDFCLLNEKSQIFVHAEPYAVSDYVNQYVGTHSIRLPKGGRPAGTLHHRIFGCLDLCRISYGGSVRVISPGLETCYHLQIILKGHCLWRGHGQEHYFAPGELLLLNPDDQADLTYSEDCEKFIVKLPSVVLDRACSDNNWHKPREGIRFAARHNLQQLDGFINLLGLVCDEAEHTKSMPRVQEHYAGIIASKLLEMLGSNVSREIFSKGNPSFERVVQFIEENLKRNISLERLAELAMMSPRSLYNLFEKHAGTTPKNYIRNRKLESIRACLNDPSANVRSITEIALDYGFLHLGRFAENYRSAFGELPSDTLRQCKKEVA

SEQ ID NO: 53 - DNA, 41 bp, Artificial
  Promoter
  misc_feature (1)..(41): P14f
gcccgttgacatgacatggttttgagggtataatgtggcga

SEQ ID NO: 54 - DNA, 41 bp, Artificial
  Promoter
  misc_feature (1)..(41): P14e
cggagttgacaacactcgaaaagccgagtataatcagatga

SEQ ID NO: 55 - DNA, 41 bp, Artificial
  Promoter
  misc_feature (1)..(41): P14d
tctacttgacatccgacattcgcgactgtataatcagatga

SEQ ID NO: 56 - DNA, 41 bp, Artificial
  Promoter
  misc_feature (1)..(41): BG19
ccaatttgacaatcaacaggaaacactttataatcagatga

SEQ ID NO: 57 - DNA, 41 bp, Artificial
  Promoter
  misc_feature (1)..(41): BG34
ataatttgacatcgaaatcgaacacatgtataatcgcttaa

SEQ ID NO: 58 - DNA, 41 bp, Artificial
  Promoter
  misc_feature (1)..(41): P14c
gtgaattgacatgtcaatttttatgttgtataatataacta

SEQ ID NO: 59 - DNA, 68 bp, Artificial
  Promoter
  misc_feature (1)..(68): PEM7
ttgttgacaattaatcatcggcatagtatatcggcatagtataatacgacaaggtgaggaactaaacc

SEQ ID NO: 60 - DNA, 35 bp, Artificial
  Promoter
  misc_feature (1)..(35): J23119
ttgacagctagctcagtcctaggtataatgctagc

SEQ ID NO: 61 - DNA, 35 bp, Artificial
  Promoter
  misc_feature (1)..(35): J23101
tttacagctagctcagtcctaggtattatgctagc

SEQ ID NO: 62 - DNA, 35 bp, Artificial
  Promoter
  misc_feature (1)..(35): J23107
tttacggctagctcagccctaggtattatgctagc

SEQ ID NO: 63 - DNA, 200 bp, Artificial
  Promoter
  misc_feature (1)..(200): JEc3
cggccgctcggtcatggcgttgtcctgcttgagcttggacccttcaggtggtcccgcgcccgcccttccgctagggcgcaagcgcgatttgcaccgcgcgccggggcttgacatgggcgtcgtgttagcgctaacatcgtcatcgacaagtgacggggggcgtgggcggcgagtcaagaccggtccaacgcgcccgccag

SEQ ID NO: 64 - DNA, 200 bp, Artificial
  Promoter
  misc_feature (1)..(200): JEa3
atgcgcccgcctcgacactcttccgtcgcggaaaccgccgcgtgtctgctatcgataccgccgcagggtgagtcccgcgcgtcggcgatgtggtcgtcagaaccaccgcttgcgcgctgataccggtaacactataatcgccggcaaccaaaggtccgacaaggaccagtagggcatcaacgggccggtacggccccatg

SEQ ID NO: 65 - DNA, 28 bp, Artificial
  Promoter
  misc_feature (1)..(28): JE1611
ttgagaattaatcatcggctcgtataat

SEQ ID NO: 66 - DNA, 200 bp, Artificial
  Promoter
  misc_feature (1)..(200): JEa2
atgcgcccgcctcgacactcttccgtcgcggaaaccgccgcgtgtctgctatcgataccgccgcagggtgagtcccgcgcgtcggcgatgtggtcgtcagaaccaccgcttgacagctgataccggtaacactaatgtcgccggcaaccaaaggtccgacaaggaccagtagggcatcaacgggccggtacggccccatg

SEQ ID NO: 67 - DNA, 28 bp, Artificial
  Promoter
  misc_feature (1)..(28): J1312
attacaattaatcatcggctcgtttaat

SEQ ID NO: 68 - DNA, 85 bp, Artificial
  Translational coupling sequence
  misc_feature (1)..(85): BCD22
gggcccaagttcacttaaaaaggagatcaacaatgaaagcaattttcgtactgaaacatcttaatcatgcctaggaagttttcta

SEQ ID NO: 69 - DNA, 85 bp, Artificial
  Translational coupling sequence
  misc_feature (1)..(85): BCD16
gggcccaagttcacttaaaaaggagatcaacaatgaaagcaattttcgtactgaaacatcttaatcatgcttaggagtctttcta

SEQ ID NO: 70 - DNA, 85 bp, Artificial
  Translational coupling sequence
  misc_feature (1)..(85): BCD23
gggcccaagttcacttaaaaaggagatcaacaatgaaagcaattttcgtactgaaacatcttaatcatgcgaaggacagtttcta

SEQ ID NO: 71 - DNA, 85 bp, Artificial
  Translational coupling sequence
  misc_feature (1)..(85): BCD8
gggcccaagttcacttaaaaaggagatcaacaatgaaagcaattttcgtactgaaacatcttaatcatgcatcggaccgtttcta

SEQ ID NO: 72 - DNA, 85 bp, Artificial
  Translational coupling sequence
  misc_feature (1)..(85): BCD21
gggcccaagttcacttaaaaaggagatcaacaatgaaagcaattttcgtactgaaacatcttaatcatgcgagggatggtttcta

SEQ ID NO: 73 - DNA, 27 bp, Artificial
  Translational coupling sequence
  misc_feature (1)..(27): MCD18
tcttaatcatgcgacggagcgtttcta

SEQ ID NO: 74 - DNA, 27 bp, Artificial
  Translational coupling sequence
  misc_feature (1)..(27): MCD8
tcttaatcatgcatcggaccgtttcta

SEQ ID NO: 75 - DNA, 27 bp, Artificial
  Translational coupling sequence
  misc_feature (1)..(27): MCD14
tcttaatcatgcggtggagggtttcta

SEQ ID NO: 76 - DNA, 27 bp, Artificial
  Translational coupling sequence
  misc_feature (1)..(27): MCD15
tcttaatcatgcgggggagtctttcta

SEQ ID NO: 77 - DNA, 27 bp, Artificial
  Translational coupling sequence
  misc_feature (1)..(27): MCD22
tcttaatcatgcctaggaagttttcta

SEQ ID NO: 78 - DNA, 85 bp, Artificial
  Translational coupling sequence
  misc_feature (1)..(85): BCD5
gggcccaagttcacttaaaaaggagatcaacaatgaaagcaattttcgtactgaaacatcttaatcatgcaggggagggtttcta

SEQ ID NO: 79 - DNA, 85 bp, Artificial
  Translational coupling sequence
  misc_feature (1)..(85): BCD9
gggcccaagttcacttaaaaaggagatcaacaatgaaagcaattttcgtactgaaacatcttaatcatgcagaggagtctttcta

SEQ ID NO: 80 - DNA, 86 bp, Artificial
  Translational coupling sequence
  misc_feature (1)..(86): BCD12
gggcccaagttcacttaaaaaggagatcaacaatgaaagcaattttcgtactgaaacatcttaatcatgcgggggagtgtttctaa

SEQ ID NO: 81 - DNA, 86 bp, Artificial
  Translational coupling sequence
  misc_feature (1)..(86): BCD13
gggcccaagttcacttaaaaaggagatcaacaatgaaagcaattttcgtactgaaacatcttaatcatgcaatggaggctttctaa

SEQ ID NO: 82 - DNA, 86 bp, Artificial
  Translational coupling sequence
  misc_feature (1)..(86): BCD19
gggcccaagttcacttaaaaaggagatcaacaatgaaagcaattttcgtactgaaacatcttaatcatgctatggaggttttctaa

SEQ ID NO: 83 - DNA, 86 bp, Artificial
  Translational coupling sequence
  misc_feature (1)..(86): BCD18
gggcccaagttcacttaaaaaggagatcaacaatgaaagcaattttcgtactgaaacatcttaatcatgcgacggagcgtttctaa

SEQ ID NO: 84 - DNA, 86 bp, Artificial
  Translational coupling sequence
  misc_feature (1)..(86): BCD20
gggcccaagttcacttaaaaaggagatcaacaatgaaagcaattttcgtactgaaacatcttaatcatgctgaggaaagtttctaa